(19) United States
(12) Patent Application Publication
(10) Pub. No.: US 2007/0049592 A1
(43) Pub. Date: Mar. 1, 2007

(54) Bis-aryl urea compounds and methods of use

(76) Inventors: Stephanie D. Geuns-Meyer, Medford, MA (US); Stuart C. Chaffee, Arlington, MA (US); Rebecca E. Johnson, Alexandria, VA (US); Joseph L. Kim, Wayland, MA (US); Joseph J. Nunes, Andover, MA (US); Vinod F. Patel, Acton, MA (US)

Correspondence Address: AMGEN INC., MAIL STOP 28-2-C, ONE AMGEN CENTER DRIVE, THOUSAND OAKS, CA, 91320-1799, US

(21) Appl. No.: 11/506,693

(22) Filed: Aug. 18, 2006

Related U.S. Application Data
(60) Provisional application No. 60/710,449, filed on Aug. 22, 2005.

(51) Int. Cl. A61K 31/5377; A61K 31/53; A61K 31/506; A61K 31/4709; C07D 413/02; C07D 403/02; C07D 401/02

(52) U.S. Cl. 514/235.5; 514/275; 514/241; 514/314; 514/352; 544/122; 544/207; 544/209; 544/329; 546/169

(57) ABSTRACT

The present invention relates to compounds of Formulas I and II, 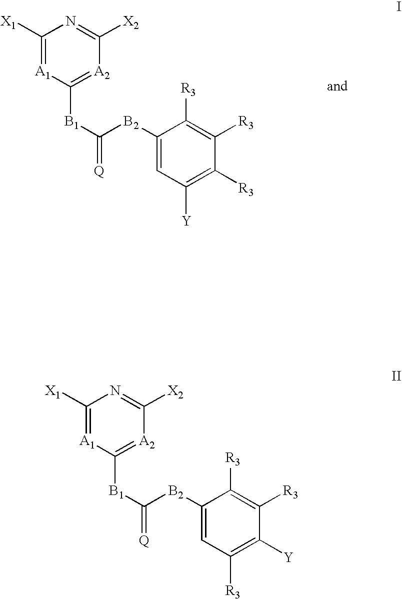
wherein A¹, A², B¹, B², Q, X¹, X², Y and R³ are defined herein, synthetic intermediates, and pharmaceutical compositions, comprising such compounds. The compounds and compositions are capable of modulating various protein kinase receptors such as Tie-2 and, therefore, influencing kinase related disease states and conditions. The compounds, for example, are capable of treating cancer caused by unregulated angiogenesis, and inflammation as well as other proliferative disorders.

This application claims the benefit of U.S. Provisional Application No. 60/710,449, filed Aug. 22, 2005, which is hereby incorporated by reference.

### FIELD OF THE INVENTION

The invention generally relates to the field of pharmaceutical agents and, more specifically, to compounds, intermediates, methods for making the compounds and intermediates, compositions, uses and methods for modulating protein kinases and for treating protein kinase-mediated diseases.

### BACKGROUND OF THE INVENTION

Protein kinases represent a large family of enzymes, which catalyze the phosphorylation of target protein substrates. The phosphorylation is usually a transfer reaction of a phosphate group from ATP to the protein substrate. Common points of attachment for the phosphate group to the protein substrate include, for example, a tyrosine, serine or threonine residue. For example, protein tyrosine kinases (PTKs) are enzymes, which catalyze the phosphorylation of specific tyrosine residues in cellular proteins. Examples of kinases in the protein kinase family include, without limitation, ab1, Akt, bcr-ab1, Blk, Brk, Btk, c-kit, c-Met, c-src, c-fms, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, flt-1, Fps, Frk, Fyn, Hck, IGF-1R, INS-R, Jak, KDR, Lck, Lyn, MEK, p38, PDGFR, PIK, PKC, PYK2, ros, tie, tie2, TRK, Yes, and Zap70. Due to their activity in numerous cellular processes, protein kinases have emerged as important therapeutic targets.

Protein kinases play a central role in the regulation and maintenance of a wide variety of cellular processes and cellular function. For example, kinase activity acts as molecular switches regulating cell proliferation, activation, and/or differentiation. Uncontrolled or excessive kinase activity has been observed in many disease states including benign and malignant proliferation disorders as well as diseases resulting from inappropriate activation of the immune system (autoimmune disorders), allograff rejection, and graft vs host disease. In addition, endothelial cell specific receptor PTKs, such as VEGF-2 and Tie-2, mediate the angiogenic process and are involved in supporting the progression of cancers and other diseases involving uncontrolled vascularization.

Angiogenesis is the process of developing new blood vessels, particularly capillaries, from pre-existing vasculature and is an essential component of embryogenesis, normal physiological growth, repair, and tumor expansion. Angiogenesis remodels small vessels into larger conduit vessels, a physiologically important aspect of vascular growth in adult tissues. Vascular growth is required for beneficial processes such as tissue repair, wound healing, recovery from tissue ischemia and menstrual cycling.

Certain diseases and/or pathological conditions develop as a result of, or are known to be associated with, the regulation and/or deregulation of angiogenesis. For example, ocular neovascularisation such as retinopathies (including diabetic retinopathy), age-related macular degeneration, psoriasis, hemangioblastoma, hemangioma, and arteriosclerosis have been found to be caused, in part, due the loss of regulation and/or maintenance of vascular growth. Inflammatory diseases such as a rheumatoid or rheumatic inflammatory disease, and especially arthritis (including rheumatoid arthritis) where new capillary blood vessels invade the joint and destroy cartilage, have been associated with angiogenesis. In addition, chronic inflammatory disorders such as chronic asthma, arterial or post-transplantational atherosclerosis, endometriosis, and neoplastic diseases including so-called solid tumors and liquid tumors (for example, leukemias), have been found to be linked to the regulation and control of angiogenesis.

The involvement of angiogenesis in major diseases has led to the identification and development of various targets for inhibiting angiogenesis. These targets relate to various receptors, enzymes, and other proteins in the angiogenic process or cascade of events leading to angiogenesis, such as, for example, activation of endothelial cells by an angiogenic signal, synthesis and release of degradative enzymes, endothelial cell migration, proliferation of endothelial cells, and formation of capillary tubules.

One target identified in the cascade of events leading to angiogenesis is the Tie receptor family. The Tie-1 and Tie-2 receptors are single-transmembrane, tyrosine kinase receptors (Tie stands for tyrosine kinase receptors with immunoglobulin and EGF homology domains). Tie-2 is an endothelial cell specific receptor tyrosine kinase, which is involved in angiogenic processes, such as vessel branching, sprouting, remodeling, maturation and stability. Tie-2 is the first mammalian receptor for which both agonist ligand(s) (for example, Angiopoietin-1 (“Ang1”) which binds to and stimulates phosphorylation and signal transduction of Tie-2), and context dependent agonist/antagonist ligand(s) (for example, Angiopoietin-2 (“Ang2”)) have been identified. Knock out and transgenic manipulation of the expression of Tie-2 and its ligands indicates that tight spacial and temporal control of Tie-2 signaling is important for the proper development of new vascularization.

Biological models suggest that the stimulation of Tie-2 by the Ang1 ligand is directly involved in the branching, sprouting and outgrowth of new vessels, and recruitment and interaction of periendothelial support cells important in maintaining vessel integrity and inducing quiescence. The absence of Ang1 stimulation of Tie-2 or the inhibition of Tie-2 autophosphorylation by Ang2, which is produced at high levels at sites of vascular regression, may cause a loss in vascular structure and matrix contacts resulting in endothelial death, especially in the absence of growth/survival stimuli.

Recently, upregulation of Tie-2 expression has been found in the vascular synovial pannus of arthritic joints of humans, consistent with the role in inappropriate neovasculariation. This finding suggests that Tie-2 plays a role in the progression of rheumatoid arthritis. Point mutations producing constitutively activated forms of Tie-2 have been identified in association with human venous malformation disorders. Tie-2 inhibitors would, therefore, be useful in treating such disorders, as well as in other instances of improper neovasacularization. However, with the recent recognition of Ang3 and Ang4 as additional Tie-2 binding ligands, targeting a Tie-2 ligand-receptor interaction as an anti-angiogenic therapeutic approach is less favorable. Accordingly, a Tie-2 receptor kinase inhibition approach has become the strategy of choice.

Another angiogenic factor responsible for regulating the growth and differentiation of the vascular system and its components, both during embryonic development and normal growth, as well as in a wide number of pathological anomalies and diseases, is Vascular Endothelial Growth Factor (“VEGF”; originally termed “Vascular Permeability Factor”, VPF), along with its cellular receptors (see G. Breier et al., Trends in Cell Biology, 6:454-456 (1996)).

VEGF is a dimeric, disulfide-linked 46-kDa glycoprotein related to “Platelet-Derived Growth Factor” (PDGF). It is produced by normal cell lines and tumor cell lines; is an endothelial cell-specific mitogen; shows angiogenic activity in in vivo test systems (e.g. rabbit cornea); is chemotactic for endothelial cells and monocytes; and induces plasminogen activators in endothelial cells, which are involved in the proteolytic degradation of extracellular matrix during the formation of capillaries. A number of isoforms of VEGF are known, which show comparable biological activity, but differ in the type of cells that secrete them and in their heparin-binding capacity. In addition, there are other members of the VEGF family, such as “Placenta Growth Factor” (PlGF) and VEGF-C.

VEGF receptors (VEGFR) are also transmembrane receptor tyrosine kinases. They are characterized by an extracellular domain with seven immunoglobulin-like domains and an intracellular tyrosine kinase domain. Various types of VEGF receptor are known, e.g. VEGFR-1 (also known as flt-1), VEGFR-2 (also known as KDR), and VEGFR-3.

A large number of human tumors, especially gliomas and carcinomas, express high levels of VEGF and its receptors. This has led to the belief that the VEGF released by tumor cells stimulates the growth of blood capillaries and the proliferation of tumor endothelium in a paracrine manner, and through the improved blood supply, accelerate tumor growth. Increased VEGF expression could explain the occurrence of cerebral edema in patients with glioma. Direct evidence of the role of VEGF as a tumor angiogenesis factor in vivo has been shown in studies in which VEGF expression or VEGF activity was inhibited. This was achieved with anti-VEGF antibodies, with dominant-negative VEGFR-2 mutants, which inhibited signal transduction, and with antisense-VEGF RNA techniques. All approaches led to a reduction in the growth of glioma cell lines or other tumor cell lines in vivo as a result of inhibited tumor angiogenesis.

Inflammatory cytokines stimulate VEGF production. Hypoxia results in a marked upregulation of VEGF in numerous tissues, hence situations involving infarct, occlusion, ischemia, anemia, or circulatory impairment typically invoke VEGF/VPF-mediated responses. Vascular hyperpermeability, associated edema, altered transendothelial exchange and macromolecular extravasation, which is often accompanied by diapedesis, can result in excessive matrix deposition, aberrant stromal proliferation, fibrosis, etc. Hence, VEGF-mediated hyperpermeability can significantly contribute to disorders with these etiologic features. As such, the regulation of angiogenesis via the VEGF receptor activity has become an important therapeutic target.

Angiogenesis is regarded as an important prerequisite for tumors that grow beyond a diameter of about 1-2 mm. Up to this size, oxygen and nutrients may be supplied to the tumor cells by diffusion. Every tumor, regardless of its origin and its cause, is thus dependent on angiogenesis for its growth after it has reached a certain size.

Three principal mechanisms play an important part in the activity of angiogenesis inhibitors against tumors: 1) inhibition of the growth of vessels, especially capillaries, into vascular resting tumors, with the result that there is no net tumor growth owing to the balance that is achieved between cell death and proliferation; 2) prevention of the migration of tumor cells owing to the absence of blood flow to and from tumors; and 3) inhibition of endothelial cell proliferation, thus avoiding the paracrine growth-stimulating effect exerted on the surrounding tissue by the endothelial cells which normally line the vessels. See R. Connell and J. Beebe, Exp. Opin. Ther. Patents, 11:77-114 (2001).

The inhibition of vascular growth in this context has also shown beneficial effects in preclinical animal models. For example, inhibition of angiogenesis by blocking vascular endothelial growth factor or its receptor has resulted in inhibition of tumor growth and in retinopathy. Also, the development of pathological pannus tissue in rheumatoid arthritis involves angiogenesis and might be blocked by inhibitors of angiogenesis.

The ability to stimulate vascular growth has potential utility for treatment of ischemia-induced pathologies such as myocardial infarction, coronary artery disease, peripheral vascular disease, and stroke. The sprouting of new vessels and/or the expansion of small vessels in ischemic tissues prevents ischemic tissue death and induces tissue repair. Regulating angiogenesis by inhibiting certain recognized pathways in this process would, therefore, be useful in treating diseases, such as ocular neovascularization, including retinopathy, age-related macular degeneration, psoriasis, hemangioblastoma, hemangioma, arteriosclerosis, inflammatory disease rheumatoid arthritis, chronic inflammatory disorders such as chronic asthma, arterial or post-transplantational atherosclerosis, endometriosis, and neoplastic diseases such as leukemias, otherwise known to be associated with deregulated angiogenesis. Treatment of malaria and related viral diseases may also be mediated by HGF and cMet.

Many classes of compounds have been proposed to treat cancerous conditions and disorders, various of them disclosing compounds to modulate or specifically inhibit Tie-2 and/or KDR kinase activity. For example, the PCT publication, WO 04/030635, published on Apr. 15, 2004, describes various classes of compounds as vasculostatic agents; PCT publication, WO 04/013141, published on Feb. 12, 2004, describes condensed pyridines and pyrimidines with Tie-2 activity; PCT publication, WO 04/054585, published on Jul. 1, 2004, describes anilino-substituted heterocyclic compounds for the treatment of abnormal cell growth; U.S. Pat. No. 6,395,733, issued May 28, 2002, describes heterocyclic ring-fused pyrimidine derivatives, useful in the treatment of hyperpoliferative diseases; U.S. Pat. No. 6,465,449, issued Oct. 15, 2002, describes heteroaromatic bicyclic derivatives useful as anticancer agents; and U.S. Patent Publication No. 2003/0018029, published Jan. 23, 2003, describes heterocyclic compounds useful in the treatment of poliferative diseases such as cancer.

### BRIEF DESCRIPTION OF THE INVENTION

The present invention provides new bis-aryl urea compounds useful in treating pathological conditions and/or disease states related to Tie-2, Lck, p38 and/or KDR kinase activity. Particularly, the compounds are useful for treating various diseases, such as cancer, inflammation and related disorders and conditions including rheumatoid arthritis. The compounds are useful by virtue of their ability to regulate active angiogenesis, cell-signal transduction and related pathways, for example, through kinase modulation. The compounds provided by the invention, including stereoisomers, tautomers, solvates, pharmaceutically acceptable salts, derivatives or prodrugs thereof, are defined by general Formula I and by Formula II 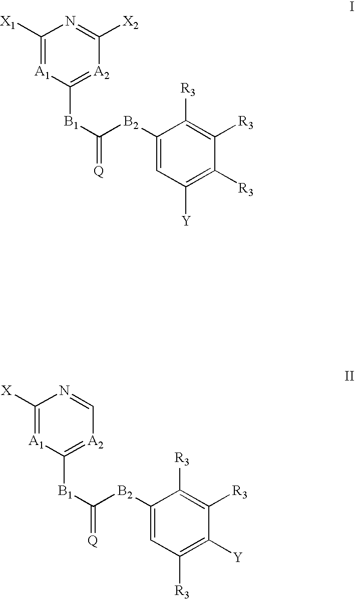
wherein A¹, A², B¹, B², Q, X¹, X², Y and R³ of Formulas I and II are as described herein below.

The invention also provides procedures for making compounds of Formula I and Formula II, as well as intermediates useful in such procedures.

The compounds provided by the invention are capable of modulating various kinase activity. For example, in one embodiment, the compounds are capable of modulating one or more kinase enzymes, such as Tie-2, Lck, KDR and P38.

To this end, the invention further provides for the use of these compounds for therapeutic, prophylactic, acute and/or chronic treatment of kinase mediated diseases, such as those described herein. For example, the invention provides the use and preparation of a medicament, containing one or more of the compounds, useful to attenuate, alleviate, or treat disorders through inhibition of Tie-2, Lck, KDR and/or P38. These compounds are also useful in the treatment of an angiogenesis- or T-cell activation- or proliferation-mediated disease or condition. Accordingly, these compounds are useful in the manufacture of anti-cancer and anti-inflammation medicaments. In one embodiment, the invention provides a pharmaceutical composition comprising an effective dosage amount of a compound of Formula I in association with a least one pharmaceutically acceptable carrier, adjuvant or diluent.

Further, the invention provides a method of treating kinase mediated disorders, such as treating angiogenesis related or T-cell activation related disorders in a subject inflicted with, or susceptible to, such disorder. The method comprises administering to the subject an effective dosage amount of a compound of Formula I. In other embodiments, the invention provides methods of reducing tumor size, blood flow to and from a tumor, and treating or alleviating various inflammatory responses, including arthritis, organ transplantation or rejection, and many others as described herein.

The foregoing merely summarizes certain aspects of the invention and is not intended, nor should it be construed, as limiting the invention in any way. All patents and other publications recited herein are hereby incorporated by reference in their entirety.

### DETAILED DESCRIPTION OF THE INVENTION

In one embodiment of the present invention, bis-aryl urea compounds of Formulas I and II, useful for treating angiogenesis- and/or T-cell proliferation-related disorders including cancer and inflammation, are provided. In one embodiment, the compounds, including stereoisomers, tautomers, solvates, pharmaceutically acceptable salts, derivatives or prodrugs thereof, are defined by general Formula I: 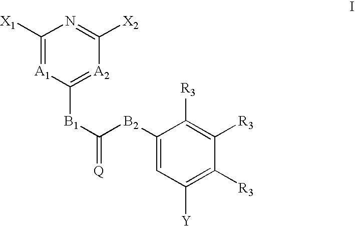
or stereoisomer, tautomer, solvate, pharmaceutically acceptable salt, derivative or prodrug thereof, wherein

A¹ is CH or N;

A² is CH or N;

B¹ is NH, NR², O or S;

B² is NH, NR², O or S;

Q is O, S, NH or N(CN);

one X¹ and X² is H, halo, NO₂, CN, NR¹R², NH₂, OR¹, SR¹, C(O)NR¹R², C(O)R⁶ or (CH₂)ₙR⁶ and the other of X¹ and X² is H;

alternatively, when A¹ is C and X¹ is N or CH, then A¹ and X¹ taken together may form a 5-6-membered unsaturated ring formed of carbons atoms and optionally comprising 1-3 heteroatoms selected from N, O and S, said ring optionally substituted with 1-3 substituents of R⁶, provided that the fused hetero bicyclic ring thus formed is not quinoline or 1,5-naphthydrine;

Y is C(O)R⁵, S(O)₂R⁵, NR⁴R⁵, C(O)NR⁴R⁴, C(O)NR⁴R⁵, COOR⁵, NR⁴C(O)R⁵, S(O)₂NR⁴R⁴, S(O)₂NR⁴R⁵ or NR⁴S(O)₂R⁵;

R¹ is C₁₋₁₀-alkyl, C₂₋₁₀-alkenyl, C₂₋₁₀-alkynyl or C₃₋₇-cycloalkyl, each of the C₁₋₁₀-alkyl, C₂₋₁₀-alkenyl, C₂₋₁₀-alkynyl and C₃₋₇-cycloalkyl optionally substituted with one or more substituents of R⁶, or R¹ is R⁶;

R² is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;

each R³, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁵ or R⁶;

alternatively any two adjacent R³'s taken together form a saturated or partially or fully unsaturated 5-6 membered monocyclic ring of carbon atoms optionally including 1-3 heteroatoms selected from O, N, or S, the ring optionally substituted independently with 1-3 substituents of R⁵ or R⁶;

each R⁴, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;

R⁵ is a partially or fully saturated or unsaturated 3-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, and wherein each ring of said ring system is optionally substituted independently with 1-3 substituents of R⁶, oxo, NR⁶R⁶, OR⁶, SR⁶, C(O)R⁶, COOR⁶, C(O)NR⁶R⁶, NR⁶C(O)R⁶, NR⁶C(O)NR⁶R⁶, OC(O)NR⁶R⁶, S(O)₂R⁶, S(O)₂NR⁶R⁶ or NR⁶S(O)₂R⁶;

each R⁶, independently, is H, oxo, halo, haloalkyl, CN, OH, NO₂, NH₂, acetyl, C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl or a saturated or partially or fully unsaturated 3-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, wherein each of the C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl and ring of said ring system is optionally substituted independently with 1-3 substituents of halo, haloalkyl, CN, NO₂, NH₂, OH, oxo, methyl, methoxyl, ethyl, ethoxyl, propyl, propoxyl, isopropyl, cyclopropyl, butyl, isobutyl, tert-butyl, methylamine, dimethylamine, ethylamine, diethylamine, propylamine, isopropylamine, dipropylamine, diisopropylamine, benzyl or phenyl; and

n is 0, 1, 2, 3 or 4.

In another embodiment, the compounds, including stereoisomers, tautomers, solvates, pharmaceutically acceptable salts, derivatives or prodrugs thereof, are defined by general Formula II: 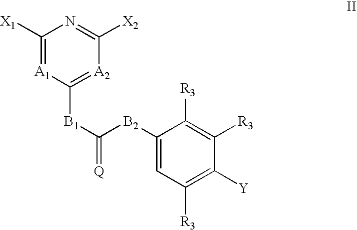
stereoisomer, tautomer, solvate, pharmaceutically acceptable salt, derivative or prodrug thereof, wherein

A¹ is CH or N;

A² is CH or N;

B¹ is NH, NR², O or S;

B² is NH, NR², O or S;

Q is O, S, NH or N(CN);

one X¹ and X² is H, halo, NO₂, CN, NR¹R², NH₂, OR¹, SR¹, C(O)NR¹R², C(O)R⁶ or (CH₂)ₙR⁶ and the other of X¹ and X² is H;

alternatively, when A¹ is C and X¹ is N or CH, then A¹ and X¹ taken together may form a 5-6-membered unsaturated ring formed of carbons atoms and optionally comprising 1-3 heteroatoms selected from N, O and S, said ring optionally substituted with 1-3 substituents of R⁶;

Y is C(O)R⁵, S(O)₂R⁵, NR⁴R⁵, C(O)NR⁴R⁴, C(O)NR⁴R⁵, COOR⁵, NR⁴C(O)R⁵, S(O)₂NR⁴R⁴, S(O)₂NR⁴R⁵ or NR⁴S(O)₂R⁵;

R¹ is C₁₋₁₀-alkyl, C₂₋₁₀-alkenyl, C₂₋₁₀-alkynyl or C₃₋₇-cycloalkyl, each of the C₁₋₁₀-alkyl, C₂₋₁₀-alkenyl, C₂₋₁₀-alkynyl and C₃₋₇-cycloalkyl optionally substituted with one or more substituents of R⁶, or R¹ is R⁶;

R² is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;

each R³, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁵ or R⁶;

alternatively any two adjacent R³'s taken together form a saturated or partially or fully unsaturated 5-6 membered monocyclic ring of carbon atoms optionally including 1-3 heteroatoms selected from O, N, or S, the ring optionally substituted independently with 1-3 substituents of R⁵ or R⁶;

each R⁴, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;

R⁵ is a partially or fully saturated or unsaturated 5-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, and wherein each ring of said ring system is optionally substituted independently with 1-3 substituents of R⁶, oxo, NR⁶R⁶, OR⁶, SR⁶, C(O)R⁶, COOR⁶, C(O)NR⁶R⁶, NR⁶C(O)R⁶, NR⁶C(O)NR⁶R⁶, OC(O)NR⁶R⁶, S(O)₂R⁶, S(O)₂NR⁶R⁶ or NR⁶S(O)₂R⁶;

each R⁶, independently, is H, oxo, halo, haloalkyl, CN, OH, NO₂, NH₂, acetyl, C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl or a saturated or partially or fully unsaturated 5-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, wherein each of the C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl and ring of said ring system is optionally substituted independently with 1-3 substituents of halo, haloalkyl, CN, NO₂, NH₂, OH, oxo, acetyl, methyl, methoxyl, ethyl, ethoxyl, propyl, propoxyl, isopropyl, cyclopropyl, butyl, isobutyl, tert-butyl, methylamine, dimethylamine, ethylamine, diethylamine, propylamine, isopropylamine, dipropylamine, diisopropylamine, benzyl or phenyl; and

n is 0, 1, 2, 3 or 4,

provided that when Y is C(O)NR⁴R⁵ and R⁵ is phenyl, then the phenyl ring is not di-meta substituted with C(O)NR⁶R⁶.

In another embodiment, the compounds of Formula I or II include N as A¹, in conjunction with any of the above or below embodiments.

In another embodiment, the compounds of Formula I or II include N as A¹ and CH as A², in conjunction with any of the above or below embodiments.

In another embodiment, the compounds of Formula I or II include N as A², in conjunction with any of the above or below embodiments.

In another embodiment, the compounds of Formula I or II include CH as A¹ and N as A², in conjunction with any of the above or below embodiments.

In another embodiment, the compounds of Formula I or II include N, independently, as both A¹ and A², in conjunction with any of the above or below embodiments.

In another embodiment, the compounds of Formula I or II include CH, independently, as both A¹ and A², in conjunction with any of the above or below embodiments.

In another embodiment, the compounds of Formula I or II include B¹ as NR², in conjunction with any of the above or below embodiments.

In another embodiment, the compounds of Formula I or II include B¹ as NR² wherein R² is an optionally substituted C₁₋₆ alkyl, in conjunction with any of the above or below embodiments.

In another embodiment, the compounds of Formula I or II include B² as NH, in conjunction with any of the above or below embodiments.

In another embodiment, the compounds of Formula I or II include Q as O, B¹ as NR² and B² as NH, in conjunction with any of the above or below embodiments.

In another embodiment, the compounds of Formula I or II include one of X¹ and X² as NR¹R² or NH₂ and the other of X¹ and X² as H, in conjunction with any of the above or below embodiments.

In another embodiment, the compounds of Formula I or II include X¹ as NR¹R² or NH₂, in conjunction with any of the above or below embodiments.

In another embodiment, the compounds of Formula I or II include X¹ as C(O)NR¹R², in conjunction with any of the above or below embodiments.

In another embodiment, the compounds of Formula I or II include X² as NR¹R² or NH₂, in conjunction with any of the above or below embodiments.

In another embodiment, the compounds of Formula I or II include both of X¹ and X² as H, in conjunction with any of the above or below embodiments.

In another embodiment, the compounds of Formula I or II include Y as NR⁴R⁵, C(O)NR⁴R⁴, C(O)NR⁴R⁵, NR⁴C(O)R⁵, S(O)₂NR⁴R⁴, S(O)₂NR⁴R⁵ or NR⁴S(O)₂R⁵, in conjunction with any of the above or below embodiments.

In another embodiment, the compounds of Formula I or II include phenyl, naphthyl, pyridyl, pyrimidyl, pyridazinyl, pyrazinyl, triazinyl, quinolinyl, isoquinolinyl, quinazolinyl, isoquinazolinyl, aza-quinazolinyl, phthalazinyl, aza-phthalazinyl, thiophenyl, furyl, pyrrolyl, pyrazolyl, imidazolyl, triazolyl, thiazolyl, oxazolyl, isoxazolyl, isothiazolyl, indolyl, isoindolyl, indolinyl, benzofuranyl, benzothiophenyl, benzimidazolyl, benzoxazolyl, benzisoxazolyl, benzothiazolyl, benzoisothiazolyl, benzotriazolyl, tetrahydrofuranyl, pyrrolidinyl, oxazolinyl, isoxazolinyl, thiazolinyl, pyrazolinyl, morpholinyl, piperidinyl, piperazinyl, pyranyl, dioxozinyl, cyclopropyl, cyclobutyl, cyclopentyl, cyclohexyl and cycloheptyl, each ring of which is optionally substituted independently with one or more substituents of R⁶, as the substituted ring of R⁵, in conjunction with any of the above or below embodiments.

In another embodiment, the compounds of Formula I or II include phenyl, naphthyl, pyridyl, piperazinyl, triazinyl, quinolinyl, isoquinolinyl, quinazolinyl, isoquinazolinyl, thiophenyl, furyl, pyrrolyl, imidazolyl, triazolyl, thiazolyl, oxazolyl, isoxazolyl, isothiazolyl, indolyl, isoindolyl, benzofuranyl, dihydrobenzofuranyl, benzothiophenyl or benzimidazolyl, each of which is optionally substituted independently with 1-3 substituents of R⁶, as R⁵ in conjunction with any of the above or below embodiments.

In another embodiment, the compounds of Formula I or II include phenyl, naphthyl, 5,6,7,8-tetrahydronaphthyl, dihydro-indenyl, pyridyl, pyrimidinyl, triazinyl, quinolinyl, tetrahydroquinolinyl, oxo-tetrahydroquinolinyl, isoquinolinyl, oxo-tetrahydroisoquinolinyl, tetrahydroisoquinolinyl, quinazolinyl, isoquinazolinyl, thiophenyl, furyl, tetrahydrofuranyl, pyrrolyl, pyrazolyl, thieno-pyrazolyl, tetrahydropentapyrazolyl, imidazolyl, triazolyl, tetrazolyl, thiazolyl, thiadiazolyl, benzothiazolyl, oxazolyl, oxadiazolyl, benzoxazolyl, benzoxadiazolyl, isoxazolyl, isothiazolyl, indolyl, azaindolyl, 2,3-dihydroindolyl, isoindolyl, indazolyl, benzofuranyl, benzothiophenyl, benzimidazolyl, imidazo-pyridinyl, purinyl, benzotriazolyl, oxazolinyl, isoxazolinyl, thiazolinyl, pyrrolidinyl, pyrazolinyl, morpholinyl, piperidinyl, piperazinyl, pyranyl, dioxozinyl, 2,3-dihydro-1,4-benzoxazinyl, 1,3-benzodioxolyl, cyclopropyl, cyclobutyl, azetidinyl, cyclopentyl, cyclohexyl and cycloheptyl, wherein said ring optionally substituted independently with 1-3 substituents of R⁶, as R⁵ in conjunction with any of the above or below embodiments.

In other embodiments, Formulas I and II include the various of the exemplary compounds described in the experimentals methods section hereinbelow.

### DEFINITIONS

The following definitions should assist in understanding the invention described herein.

The terms “agonist” and “agonistic” when used herein refer to or describe a molecule which is capable of, directly or indirectly, substantially inducing, promoting or enhancing biological activity of a biological molecule, such as an enzyme or receptor, including Tie-2 and Lck.

The term “comprising” is meant to be open ended, including the indicated component(s), but not excluding other elements.

The term “H” denotes a single hydrogen atom. This radical may be attached, for example, to an oxygen atom to form a hydroxyl radical.

The term “C_{α-β}alkyl”, when used either alone or within other terms such as “haloalkyl” and “alkylamino”, embraces linear or branched radicals having α to β number of carbon atoms (such as C₁-C₁₀). The term “alkyl” radicals include “lower alkyl” radicals having one to about six carbon atoms. Examples of such radicals include methyl, ethyl, n-propyl, isopropyl, n-butyl, isobutyl, sec-butyl, tert-butyl, pentyl, isoamyl, hexyl and the like. The term “alkylenyl” embraces bridging divalent alkyl radicals such as methylenyl and ethylenyl.

The term “alkenyl”, when used alone or in combination, embraces linear or branched radicals having at least one carbon-carbon double bond in a moiety having between two and ten carbon atoms. Included within alkenyl radicals are “lower alkenyll” radicals having two to about six carbon atoms and, for example, those radicals having two to about four carbon atoms. Examples of alkenyl radicals include, without limitation, ethenyl, propenyl, allyl, propenyl, butenyl and 4-methylbutenyl. The terms “alkenyl” and “lower alkenyl”, embrace radicals having “cis” and “trans” orientations, or alternatively, “E” and “Z” orientations, as appreciated by those of ordinary skill in the art.

The term “alkynyl”, when used alone or in combination, denotes linear or branched radicals having at least one carbon-carbon triple bond and having two to ten carbon atoms. Examples of alkynyl radicals include “lower alkynyl” radicals having two to about six carbon atoms and, for example, lower alkynyl radicals having two to about four carbon atoms. Examples of such radicals include, without limitation, ethynyl, propynyl (propargyl), butynyl, and the like.

The term “alkoxy” or “alkoxyl”, when used alone or in combination, embraces linear or branched oxygen-containing radicals each having alkyl portions of one or more carbon atoms. The term alkoxy radicals include “lower alkoxy” radicals having one to six carbon atoms. Examples of such radicals include methoxy, ethoxy, propoxy, butoxy and tert-butoxy. Alkoxy radicals may be further substituted with one or more halo atoms, such as fluoro, chloro or bromo, to provide “haloalkoxy” radicals. Examples of such radicals include fluoromethoxy, chloromethoxy, trifluoromethoxy, trifluoroethoxy, fluoroethoxy and fluoropropoxy.

The term “aryl”, when used alone or in combination, means a carbocyclic aromatic moiety containing one, two or even three rings wherein such rings may be attached together in a fused manner. Every ring of an “aryl” ring system need not be aromatic, and the ring(s) fused to the aromatic ring may be partially or fully unsaturated and include one or more heteroatoms selected from nitrogen, oxygen and sulfur. Thus, the term “aryl” embraces aromatic radicals such as phenyl, naphthyl, indenyl, tetrahydronaphthyl, dihydrobenzafuranyl, anthracenyl, indanyl, benzodioxazinyl, and the like. The “aryl”, group may be subsitituted, such as with 1 to 5 substituents including lower alkyl, hydroxyl, halo, haloalkyl, nitro, cyano, alkoxy and lower alkylamino, and the like. Phenyl substituted with —O—CH₂—O— or —O—CH₂—CH₂—O— forms an aryl benzodioxolyl substituent.

The term “carbocyclic”, also referred to herein as “cycloalkyl”, when used alone or in combination, means a partially or fully saturated ring moiety containing one (“monocyclic”), two (“bicyclic”) or even three (“tricyclic”) rings wherein such rings may be attached together in a fused manner and formed from carbon atoms. Examples of saturated carbocyclic radicals include saturated 3 to 6-membered monocyclic groups such as cyclopropane, cyclobutane, cyclopentane and cyclohexane.

The terms “ring” and “ring system” refer to a ring comprising the delineated number of atoms, the atoms being carbon or, where indicated, a heteroatom such as nitrogen, oxygen or sulfur. The ring itself, as well as any substitutents thereon, may be attached at any atom that allows a stable compound to be formed. The term “nonaromatic” ring or ring system refers to the fact that at least one, but not necessarily all, rings in a bicyclic or tricyclic ring system is nonaromatic.

The term “cycloalkenyl”, when used alone or in combination, means a partially or fully saturated cycloalkyl containing one, two or even three rings in a structure having at least one carbon-carbon double bond in the structure. Examples of cycloalkenyl groups include C₃-C₆ rings, such as compounds including, without limitation, cyclopropene, cyclobutene, cyclopentene and cyclohexene. The term also includes carbocyclic groups having two or more carbon-carbon double bonds such as “cycloalkyldienyl” compounds. Examples of cycloalkyldienyl groups include, without limitation, cyclopentadiene and cycloheptadiene.

The term “halo”, when used alone or in combination, means halogens such as fluorine, chlorine, bromine or iodine atoms.

The term “haloalkyl”, when used alone or in combination, embraces radicals wherein any one or more of the alkyl carbon atoms is substituted with halo as defined above. For example, this term includes monohaloalkyl, dihaloalkyl and polyhaloalkyl radicals such as a perhaloalkyl. A monohaloalkyl radical, for example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms and, for example, lower haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl”, as used herein, refers to alkyl radicals having all hydrogen atoms replaced with fluoro atoms. Examples include trifluoromethyl and pentafluoroethyl.

The term “heteroaryl”, as used herein, either alone or in combination, means a fully unsaturated (aromatic) ring moiety formed from carbon atoms and having one or more heteroatoms selected from nitrogen, oxygen and sulfur. The ring moiety or ring system may contain one (“monocyclic”), two (“bicyclic”) or even three (“tricyclic”) rings wherein such rings are attached together in a fused manner. Every ring of a “heteroaryl” ring system need not be aromatic, and the ring(s) fused thereto (to the heteroaromatic ring) may be partially or fully saturated and optionally include one or more heteroatoms selected from nitrogen, oxygen and sulfur. The term “heteroaryl” does not include rings having ring members of —O—O—, —O—S— or —S—S—.

Examples of unsaturated heteroaryl radicals, include unsaturated 5- to 6-membered heteromonocyclyl groups containing 1 to 4 nitrogen atoms, including for example, pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl] and tetrazole; unsaturated 7- to 10-membered heterobicyclyl groups containing 1 to 4 nitrogen atoms, including for example, quinolinyl, isoquinolinyl, quinazolinyl, isoquinazolinyl, aza-quinazolinyl, and the like; unsaturated 5- to 6-membered heteromonocyclic group containing an oxygen atom, for example, pyranyl, 2-furyl, 3-furyl, benzofuryl, etc.; unsaturated 5 to 6-membered heteromonocyclic group containing a sulfur atom, for example, 2-thienyl, 3-thienyl, benzothienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, isothiazolyl, thiadiazolyl [e.g., 1,2,4-thiadiazolyl, 1,3,4-thiadiazolyl, 1,2,5-thiadiazolyl].

The term “heterocyclic”, when used alone or in combination, means a partially or fully saturated ring moiety containing one, two (heterobicyclic) or even three (heterotricyclic) rings wherein such rings may be attached together in a fused manner, formed from carbon atoms and including one or more heteroatoms selected from N, O or S. Examples of saturated heterocyclic radicals include saturated 3 to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothienyl, dihydropyranyl, dihydrofuryl and dihydrothiazolyl.

The term “heterocycle” also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl]; and saturated, partially unsaturated and unsaturated condensed heterocyclic group containing 1 to 2 oxygen or sulfur atoms [e.g. benzofuryl, benzothienyl, 2,3-dihydro-benzo[1,4]dioxinyl and dihydrobenzofuryl]. Examples of heterocyclic radicals include five to ten membered fused or unfused radicals.

Examples of partially saturated and saturated heterocyclyl include, without limitation, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl, and the like.

The term “3-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S′, as used herein, means that the ring or ring system may be a carbocycle, an aryl, a heterocycle or a heteroaryl monocyclic, bicyclic or tricyclic ring or ring system.

The term “alkylamino” includes “N-alkylamino” where amino radicals are independently substituted with one alkyl radical. Preferred alkylamino radicals are “lower alkylamino” radicals having one to six carbon atoms. Even more preferred are lower alkylamino radicals having one to three carbon atoms. Examples of such lower alkylamino radicals include N-methylamino, and N-ethylamino, N-propylamino, N-isopropylamino and the like.

The term “dialkylamino” includes “N, N-dialkylamino” where amino radicals are independently substituted with two alkyl radicals. Preferred alkylamino radicals are “lower alkylamino” radicals having one to six carbon atoms. Even more preferred are lower alkylamino radicals having one to three carbon atoms. Examples of such lower alkylamino radicals include N,N-dimethylamino, N,N-diethylamino, and the like.

The terms “carboxy” or “carboxyl”, whether used alone or with other terms, such as “carboxyalkyl”, denotes —CO₂H.

The term “carbonyl”, whether used alone or with other terms, such as “aminocarbonyl”, denotes —(C═O)—.

The term “aminocarbonyl”, denotes an amide group of the formula —C(═O)NH₂.

The term “alkylthio” embraces radicals containing a linear or branched alkyl radical, of one to ten carbon atoms, attached to a divalent sulfur atom. An example of “alkylthio” is methylthio, (CH₃S—).

The term “haloalkylthio” embraces radicals containing a haloalkyl radical, of one to ten carbon atoms, attached to a divalent sulfur atom. An example of “haloalkylthio” is trifluoromethylthio.

The term “Formula I” includes any sub formulas. Similarly, the term “Formula II” includes any sub formulas.

The term “pharmaceutically-acceptable” when used with reference to a compound of Formulas I or II is intended to refer to a form of the compound that is safe for administration. For example, a salt form, a solvate, a hydrate or derivative form of a compound of Formula I or of Formula II, which has been approved for mammalian use, via oral ingestion or other routes of administration, by a governing body or regulatory agency, such as the Food and Drug Administration (FDA) of the United States, is pharmaceutically acceptable.

Included in the compounds of Formulas I and II are the pharmaceutically acceptable salt forms of the free-base compounds. The term “pharmaceutically-acceptable salts” embraces salts commonly used to form alkali metal salts and to form addition salts of free acids or free bases. As appreciated by those of ordinary skill in the art, salts may be formed from ionic associations, charge-charge interactions, covalent bonding, complexation, coordination, etc. The nature of the salt is not critical, provided that it is pharmaceutically acceptable.

Suitable pharmaceutically acceptable acid addition salts of compounds of Formulas I and II may be prepared from an inorganic acid or from an organic acid. Examples of such inorganic acids are hydrochloric, hydrobromic, hydroiodic, hydrofluoric, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which include, without limitation, formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, thiocyanic, undecanoic, stearic, algenic, β-hydroxybutyric, salicylic, galactaric and galacturonic acid. Suitable pharmaceutically-acceptable base addition salts of compounds of Formulas I and II include metallic salts, such as salts made from aluminum, calcium, lithium, magnesium, potassium, sodium and zinc, or salts made from organic bases including, without limitation, primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, disopropylethylamine and trimethylamine. All of these salts may be prepared by conventional means from the corresponding compound of the invention by reacting, for example, the appropriate acid or base with the compound of Formulas I or II.

Also, the basic nitrogen-containing groups can be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, hydrobromic acid, citric acid, sulphuric acid and phosphoric acid and such organic acids as oxalic acid, stearic and, salicylic acid, pamoic acid, gluconic acid, ethanesulfonic acid, methanesulfonic acid, toluenesulfonic acid, tartaric acid, fumaric acid, medronic acid, napsylic acid, maleic acid, succinic acid and citric acid. Other examples include salts with alkali metals or alkaline earth metals such as sodium, potassium, calcium or magnesium, or with organic bases.

Additional examples of such salts can be found in Berge et al., J. Pharm. Sci., 66, 1 (1977). Conventional methods may be used to form the salts. For example, a phosphate salt of a compound of the invention may be made by combining the desired compound free base in a desired solvent, or combination of solvents, with phosphoric acid in a desired stoichiometric amount, at a desired temperature, typically under heat (depending upon the boiling point of the solvent). The salt can be precipitated upon cooling (slow or fast) and may crystallize (i.e., if crystalline in nature), as appreciated by those of ordinary skill in the art. Further, hemi-, mono-, di, tri- and poly-salt forms of the compounds of the present invention are also contemplated herein. Similarly, hemi-, mono-, di, tri- and poly-hydrated forms of the compounds, salts and derivatives thereof, are also contemplated herein.

The term “derivative” is broadly construed herein, and intended to encompass any salt of a compound of this invention, any ester of a compound of this invention, or any other compound, which upon administration to a patient is capable of providing (directly or indirectly) a compound of this invention, or a metabolite or residue thereof, characterized by the ability to the ability to modulate a kinase enzyme.

The term “pharmaceutically-acceptable derivative” as used herein, denotes a derivative which is pharmaceutically acceptable.

The term “prodrug”, as used herein, denotes a compound which upon administration to a subject or patient is capable of providing (directly or indirectly) a compound of this invention. Examples of prodrugs would include esterified or hydroxylated compounds where the ester or hydroxyl groups would cleave in vivo, such as in the gut, to produce a compound according to Formula I. A “pharmaceutically-acceptable prodrug” as used herein, denotes a prodrug which is pharmaceutically acceptable. Pharmaceutically acceptable modifications to the compounds of Formula I are readily appreciated by those of ordinary skill in the art.

The compound(s) of Formula I or II may be used to treat a subject by administering the compound(s) as a pharmaceutical composition. To this end, the compound(s) can be combined with one or more carriers, diluents or adjuvants to form a suitable composition, which is described in more detail herein.

The term “carrier”, as used herein, denotes any pharmaceutically acceptable additive, excipient, adjuvant, or other suitable ingredient, other than the active pharmaceutical ingredient (API), which is typically included for formulation and/or administration purposes. “Diluent” and “adjuvant” are defined hereinafter.

The terms “treat”, “treating,” “treatment,” and “therapy” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

The phrase “effective dosage amount” is intended to quantify the amount of each agent, which will achieve the goal of improvement in disorder severity and the frequency of incidence over treatment of each agent by itself, while avoiding adverse side effects typically associated with alternative therapies. For example, effective neoplastic therapeutic agents prolong the survivability of the patient, inhibit the rapidly-proliferating cell growth associated with the neoplasm, or effect a regression of the neoplasm.

The term “leaving groups” generally refer to groups that are displaceable by a nucleophile. Such leaving groups are known in the art. Examples of leaving groups include, but are not limited to, halides (e.g., I, Br, F, Cl), sulfonates (e.g., mesylate, tosylate), sulfides (e.g., SCH₃), N-hydroxsuccinimide, N-hydroxybenzotriazole, and the like. Nucleophiles are species that are capable of attacking a molecule at the point of attachment of the leaving group causing displacement of the leaving group. Nucleophiles are known in the art. Examples of nucleophilic groups include, but are not limited to, amines, thiols, alcohols, Grignard reagents, anionic species (e.g., alkoxides, amides, carbanions) and the like.

The term “angiogenesis” is defined as any alteration of an existing vascular bed or the formation of new vasculature which benefits tissue perfusion. This includes the formation of new vessels by sprouting of endothelial cells from existing blood vessels or the remodeling of existing vessels to alter size, maturity, direction and/or flow properties to improve blood perfusion of tissue.

The terms “cancer” and “cancerous” when used herein refer to or describe the physiological condition in mammals that is typically characterized by unregulated cell growth. Examples of cancer include, without limitation, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer. While the term “cancer” as used herein is not limited to any one specific form of the disease, it is believed that the methods of the invention will be particularly effective for cancers which are found to be accompanied by unregulated levels of Tie-2, and similar kinases, in the mammal.

### General Synthetic Procedures

The present invention further comprises procedures for the preparation of a compound of Formulas I and II.

The compounds of Formulas I and II can be synthesized according to the procedures described in the following Schemes 1-5, wherein the substituents are as defined for Formulas I and II, above, except where further noted. The synthetic methods described below are merely exemplary, and the compounds of the invention may be synthesized by alternate routes as appreciated by persons of ordinary skill in the art. The compounds exemplified herein are named using either the IUPAC naming convention or the naming convention of MDL or ChemDraw software.

The following list of abbreviations used throughout the specification represent the following and should assist in understanding the invention:
ACN, MeCN—acetonitrile
BSA—bovine serum albumin
Cs₂CO₃—cesium carbonate
CHCl₃—chloroform
CH₂Cl₂, DCM—dichloromethane, methylene chloride
DIC—1,3-diisopropylcarbodiimide
DIEA, (iPr)₂NEt—diisopropylethylamine
DME—dimethoxyethane
DMF—dimethylformamide
DMAP—4-dimethylaminopyridine
DMSO—dimethylsulfoxide
EDC—1-(3-dimethylaminopropyl)-3-ethylcarbodiimide
Et₂O—diethyl ether
EtOAc—ethyl acetate
PyBop—benzotriazol-1-yl-oxy-tripyrrolidino-phosphonium hexafluorophosphate
RT, rt—room temperature
TBTU—O-benzotriazol-1-yl-N,N,N′,N′-tetramethyluronium tetrafluoroborate
TEA, Et₃N—triethylamine
TFA—trifluoroacetic acid
THF—tetrahydrofuran
G, gm—gram
h, hr—hour
H₂—hydrogen
HATU—O-(7-azabenzotriazol-1-yl)-N,N,N′,N′-tetramethyluroniumhexafluorophosphate
HOBt—1-hydroxybenzotriazole hydrate
HPLC—high pressure liquid chromatography
IPA, IpOH—isopropyl alcohol
K₂CO₃—potassium carbonate
MgSO₄—magnesium sulfate
MeOH—methanol
N₂—nitrogen
NaHCO₃—sodium bicarbonate
NaOCH₃—sodium methoxide
Na₂SO₄—sodium sulfate
NH₄Cl—ammonium chloride
NH₄OH—ammonium hydroxide
Pd/C—palladium on carbon

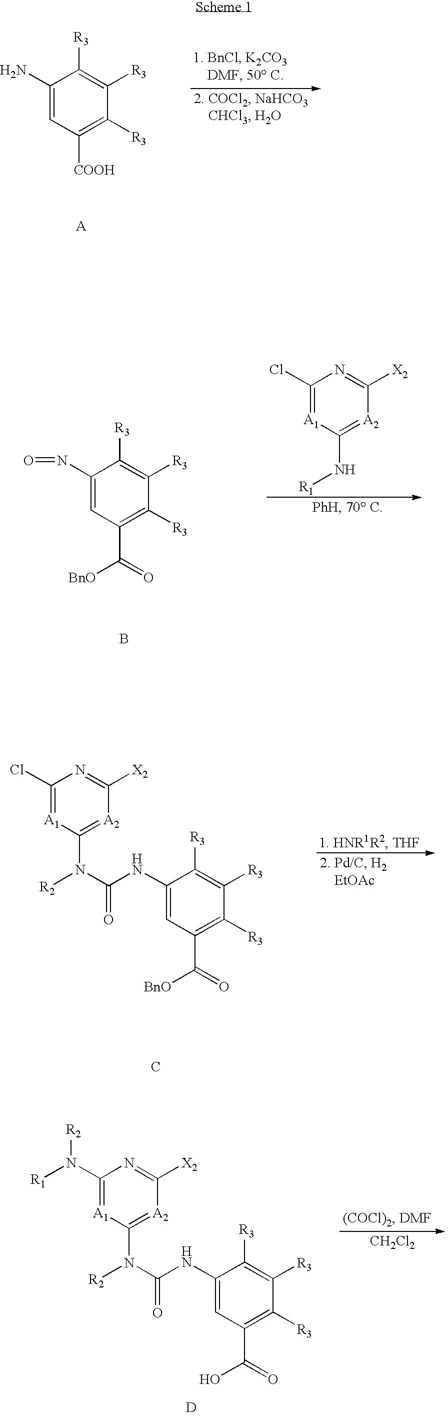

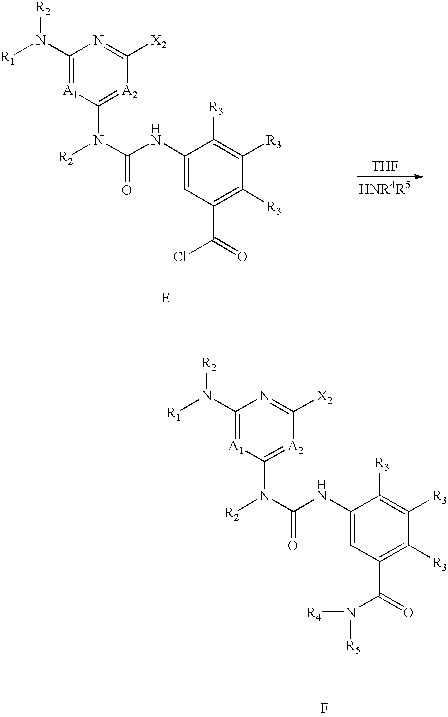

Compounds F of Formulas I and II (where the amide is para-substituted on the phenyl ring) can be made utilizing the method described in Scheme 1. As shown, a compound F may be made starting with an amino-benzoic acid A, referred to herein and throughout the specification as the “B” ring. The acid A can first be protected by known acid protecting groups, such as a benzyl group as shown above, and then the aniline A can be converted to the corresponding isocyanate B using conventional methods, such as with oxalyl chloride, as shown. The isocyanate B can be reacted with a desired chloroheterocyclic ring, referred to herein and throughout the specification as the “D” ring, to generate the resulting urea between the D and B rings, as shown in Formulas I and II. The protecting group can be removed from the B ring, and the free acid functional group of compound D can be converted to an activated group, such as an acid chloride group of compound E, and reacted with a desired amine to afford the desired product F, where Y is an amide linker between an R⁵ group and the B ring. This method allows one to prepare compounds with desireable R⁵ groups conveniently and easily.

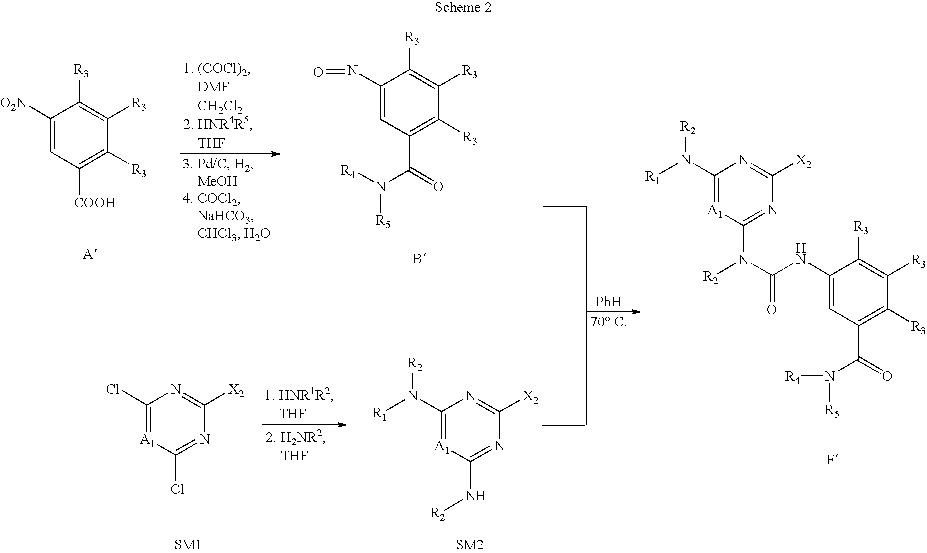

Compounds F′ of Formulas I and II can be made utilizing the method described in Scheme 2. As shown, a compound F′ may be made starting with a nitro-benzoic acid A, as the B ring. The acid A can first be activated and coupled to the desired amine, as shown in scheme 1 above. The nitro can be reduced using conventional methods, such as by hydrogenation shown above, and then the corresponding amine converted to the isocyanate B′ using conventional methods, such as that shown above in scheme 2. The isocyanate B′ can be reacted with a desired, previously functionalized D ring SM2, to generate the resulting urea F′ between the D and B rings, as shown in Formulas I and II. This is another method of preparing compounds of Formulas I and II, where the D ring and the R⁵ group may be independently modified, as desired. 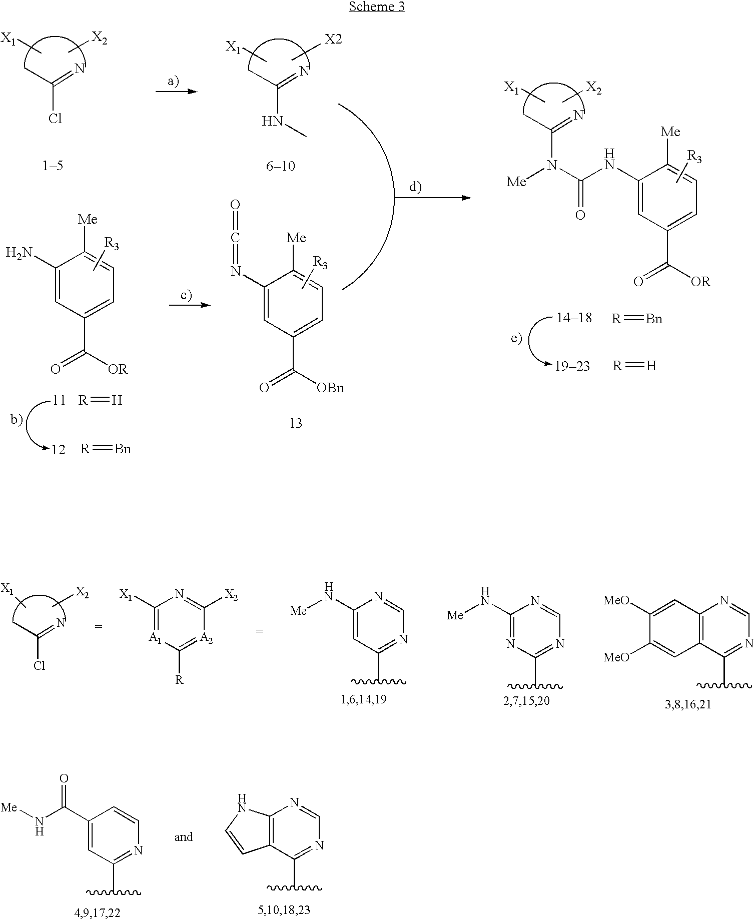
Conditions: a) MeNH₂, −10° C. to 140° C. b) benzylbromide, K2CO3, DMF, 50°. C) COCl₂, CH₂Cl₂/aq. sat. NaHCO₃, 0°->rt. d) see text. e) H₂, 10%-Pd/C, 2-methoxyethanol/dioxane at 80° C. or MeOH at rt.

Alternatively, the compounds of Formulas I and II, including exemplary compounds 14-18 and 19-23 (Scheme 4) may be synthesized beginning with the method described in Scheme 3 above. As shown, various aryl groups containing a nitrogen atom, such as starting aryl rings 1-5, may be attached to the second aryl group, the “B” ring illustrated above for purposes of an example as a phenyl ring, via a urea linker (shown above) by the method described in Scheme 3. As shown, chloro-substituted D ring starting materials 1-5 may be converted to the corresponding methylamino substitution by displacement of the chlorine with methylamine. More specifically, nucleophilic substitution of the commercially available chloroheterocycles 1-5 with methylamine at different temperatures should afford the N-methyl derivatives 6-10. Aminobenzoic acid 11 can be benzylated using benzylbromide (conditions b) to produce product 12, which can them be converted into the isocyanate 13 by conventional methods. As shown, oxalyl chloride and aqueous base as described in conditions c can be used to form the desired isocyanate 13. Addition of isocyanate 13 to a pyrimidine 6 or a triazine 7 in a suitable solvent, such as refluxing dioxane, or combination of solvents, should lead to the corresponding urea derivatives 14 and 15 in good yield (in both examples B¹═NHR¹, where R¹=methyl). The reaction of aminopyridine 9, for example, under these conditions proved to be sluggish and accordingly, weaker and/or sterically hindered nucleophiles including nucleophilic anilines may require slightly harsher conditions and higher temperatures (CHCl₃, 175°, 1 h, microwave), as appreciated by those of ordinary skill in the art. Purification by chromatography should afford the desired ureas 17 in good yield.

Depending upon the particular D ring chosen, the conditions to affect the urea formation between the D and B rings may need to vary, as appreciated by those of ordinary skill in the art. For example, different solvents (toluene, dioxane, DMF or CHCl₃) and bases (DMAP, DIPEA, K₂CO₃ or NaH) may be used, or even stoichiometrically excessive isocyanate may be required to afford the desired products 14-18. The reaction of deazapurin 10 with 13, for example, afforded the regioisomeric urea of 18 (structure not shown) as evidenced by the disappearance of the pyrrole-NH signal in the ¹H-NMR spectrum.

Compounds 14-18 may then be deprotected using conventional methods to afford the corresponding acids 19-23, which may then be used in Scheme 4 to prepare compounds of Formulas I and II. For example, condition e describes the final hydrogenolytic cleavage of the benzyl protecting group of compounds 14-18 which afforded acid building block intermediates 19-23. These building blocks were used in methods described in Scheme 4 below.

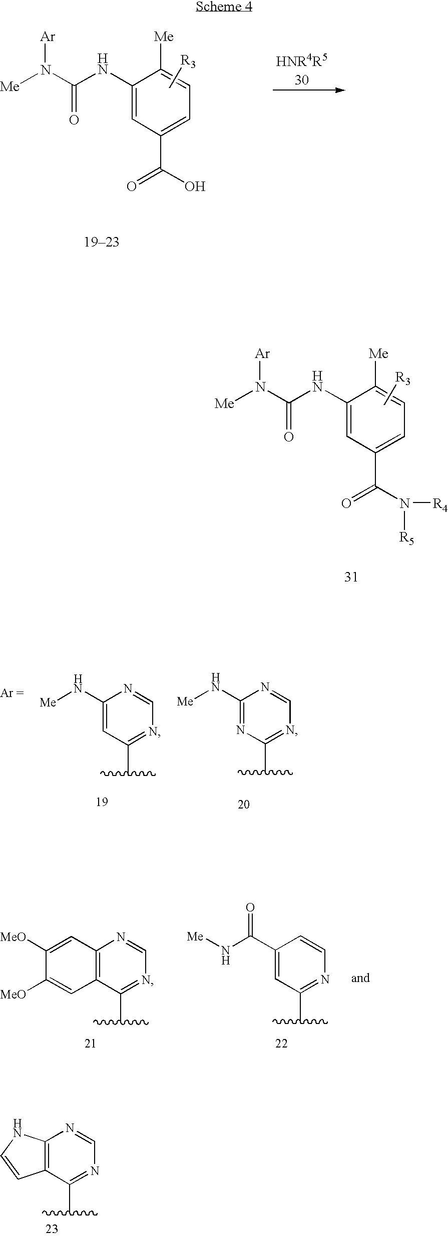

Compounds 31 (Y in Formulas I and II=—C(O)NR⁴R⁵) may be made using the method described in Scheme 4. As shown, intermediate acids 19-23 may be reacted with an amine 30 in the presence of known coupling reagents and suitable solvents to afford the desired amides 31. Suitable reaction conditions include conventional methods, known to those of ordinary skill in the art.

For example, amide, sulfonamide, urea, carbamate, and ester bond formation usually require the following reagent-comprising reactive centers: a nucleophile Nu⁻ and an electrophile E⁺, which may also be referred to as a leaving group “LG” or X. Suitable “leaving groups” include a halide (bromine, chlorine, iodine or fluorine), alkylsulfonate and other known leaving groups (also see definitions herein). Suitable nucleophiles or nucleophilic species Nu⁻ include a primary or secondary amine, an oxygen, a sulfur or a anionic carbon species. Examples of nucleophiles include, without limitation, amines, hydroxides, alkoxides and the like. Suitable electrophiles or electrophilic species E⁺, include the carbon atom of a carbonyl or carbon atom attached to an activated leaving group, the carbon atom of which is susceptible to nucleophilic attack or readily eliminates. Examples of suitable electrophilic carbonyl species include, without limitation, acid halides, mixed anhydrides, aldehydes, carbamoyl-chlorides, sulfonyl chlorides (sulfonyl electrophile), acid carbonyls activated with standard, known coupling reagents or also referred to herein as “activating reagents”, such as TBTU, HBTU, HATU, HOBT, BOP, PyBOP, carbodiimides (DCC, EDC and the like), pentafluorophenyl, and other electrophilic species including halides, isocyanates (see scheme 1), diazonium ions and the like. The protected carbonyl allows one to take a desired D-linked B ring intermediates and attach various R⁵ ring intermediates such as selected R⁴-R⁵ coupled primary or secondary amines (scheme 4 above). This allows one the advantage of modifying the R⁵ group in a single step.

The coupling of rings B and desired R⁵ rings (referred to herein and throughout the specification as the “A” ring), as shown in compounds of Formulas I and II, can be brought about using various conventional methods to link rings B and A together. For example, an amide or a sulfonamide linker where the Nu- is an amine, respectively, can be made utilizing an amine on either the B or A rings and an acid chloride or sulfonyl chloride on the other of either the A or B rings. The reaction proceeds generally in the presence of a suitable solvent and/or base. The reaction proceeds generally in the presence of a suitable solvent and/or base. Suitable solvents include, without limitation, generally non-nucleophilic, aprotic solvents such as toluene, CH₂Cl₂, THF, DMF, DMSO, N,N-dimethylacetamide and the like, and solvent combinations thereof. The solvent(s) may range in polarity, as appreciated by those skilled in the art. Suitable bases include, for example, mild bases such as tertiary amine bases including, without limitation, DIEA, TEA, N-methylmorpholine; and stronger bases such as carbonate bases including, without limitation, Na₂CO₃, K₂CO₃, Cs₂CO₃; hydrides including, without limitation, NaH, KH, borohydrides, cyanoborohydrides and the like; and alkoxides including, without limitation, NaOCH₃, and the like. The base itself may also serve as a solvent. The reaction may optionally be run neat, i.e., without any base and/or solvent. For simple structurally unhindered substrates, these coupling reactions are generally fast and conversion occurs typically in ambient conditions. However, depending upon the particular substrate, steric hindrance, concentration and other stoichiometric factors, such reactions may be sluggish and may require a basicity adjustment or heat, as appreciated by those skilled in the art.

As another example, a urea linker (or a sulfonylurea linker), as shown in scheme 3, may be made by reacting an amine with a desired isocyanate. As isocyanates are generally highly reactive species, the urea formation generally proceeds quickly, at ambient temperatures with a minimal amount of solvent, as appreciated by those of ordinary skill in the art. The reaction may optionally be run neat, i.e., without any base and/or solvent.

Similarly, carbamate linkers where Nu- would be an amine, thiourea linkers where the respective carbonyl oxygen is a sulfur, and thiocarbamates where the respective carbonyl oxygen and/or carbamate oxygen is a sulfur made be made by similar methods. While the above methods are so described, they are not exhaustive, and other methods for linking rings A and B together may be utilized as appreciated by those skilled in the art. 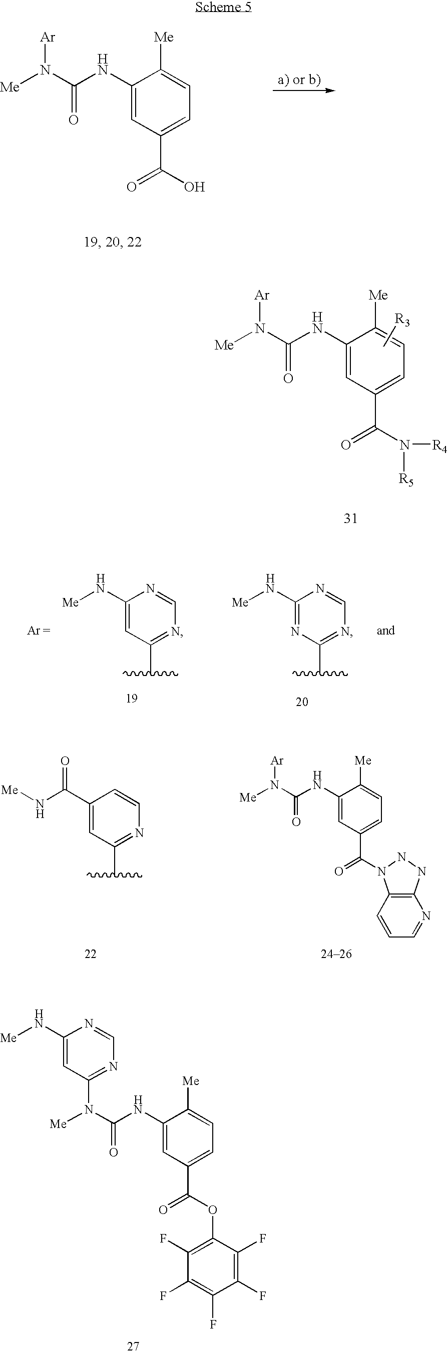
Conditions: a) HATU, HOAt, amine (30), Hunig's base, DMF, rt to 85° C. or b) COCI₂, CHCl₃, 0° C., 5 min. then DMF, 60-90 min; amine (30), CHCI3, rt to 55° C., 16 h.

Scheme 5 describes a few exemplary methods, which may be used to make amide bonds as the linker “L” for compounds of Formulas I and II. Activated carbonyl intermediates 24-26 and the corresponding pentafluorophenyl ester of acids 19, 20 and 22 may be made using known methods, as described above. More specifically, the pentafluorophenylester 27 (prepared in one step from 19) may be coupled under conventional reaction conditions, including neat reaction without solvent, reaction in a microwave apparatus, utilizing bases of differing strengths, e.g. NaH, DMAP, with strong nucleophilic anilines to afford the corresponding amide derivatives 31. Reaction of the pentafluorophenylester 27 with weak nucleophilic anilines, such as p-trifluoromethylaniline, may require stronger reaction conditions, as appreciated by those of ordinary skill in the art.

In situ generation of the corresponding acid chlorides derived from 19, 20 and 22 in a 4:1 mixture of CHCl₃/THF followed by the addition of excess aniline (3.5 Eq.) afforded additional final compounds 31, derived from the weaker nucleophilic anilines. The observed yields with the acid chloride derived from 22 were generally higher than those with the acid chloride derived from 19. This may be attributed to a improved solubility in the solvent mixture.

Alternatively, amides 31 may be synthesized using parallel synthesis techniques (not shown). The parallel synthesis may be used for more nucleophilic amines and anilines, reacted with the acids using a HATU/HOAt mediated coupling to give final products 31, in reasonable yield after purification by silica-gel chromatography or washing. The reaction with more deactivated anilines (eg. entries 19, 20, 21) did not lead to the targeted amides under these conditions; instead, the activated azobenzotriazol derivatives 24-26 were formed (Scheme 3). Running the coupling reaction at 85° C. for 7 h afforded the final anilides 31. However, prolonged heating or higher temperatures sometimes led to decomposition of the activated intermediates 24-26.

Various experimental methods have been employed to synthesize compounds of Formulas I and II, as more generally described in schemes 1-5 above, and further described in more detail by the representative examples below.

To enhance the understanding and appreciation of the present invention, the following exemplary methods and specific examples (starting reagents, intermediates and compounds of Formulas I and II) are set forth. It should be appreciated that these methods and examples are merely for illustrative purposes-only and are not to be construed as limiting the scope of this invention in any manner.

### EXAMPLE 1

Synthesis of Compound 6 in Scheme 3

To 4,6-Dichloropyrimidine (9.5 g, 63.77 mmol) in a sealable tube was slowly added cold methyl amine solution (80 ml, 640 mmol, 8 M in EtOH) at 0° C. The tube was closed and stirred for 16 h at 80° C. After cooling to rt, the formed precipitate was filtered off. To the white solid was added Na₂CO₃ (1 M, aq.) and ethyl acetate and slurry was stirred for 30 min. After filtration and drying, compound 6 was obtained as white crystals.

C₆H₁₀N₄ (138.17): TLC (CH₂CI₂/MeOH 9:1) Rf: 0.1. MS-APCI: 139 ([M+H]⁺, 70). ′HNMR (300 MHz, DMSO-d6): 6 (ppm)=7.88 (s, 1H), 6.45 (m, 1H), 5.23 (s, 1H), 2.68 (d, J=4.9, 6H).

### EXAMPLE 2

Synthesis of Compound 7 in Scheme 3

To a mixture of 2,6-dichlorotriazine (3, 6 g, 40 mmol) in dry THF (20 ml) was added methylamine (60 ml, 480 mmol, 8 M in EtOH) dropwise at −10° C. After the addition was complete, the reaction mixture was transferred into a sealed vessel and it was stirred at rt until the reaction was complete. The reaction mixture was diluted with ethyl acetate, washed with 1 M aq Na₂CO₃, dried over Na₂SO₄, filtered and concentrated. The crude residue was adsorbed on silica-gel and purified by chromatography (ethyl acetate/MeOH gradient). Further purification by washing with Et20 and ethyl acetate afforded compound 7 as a white solid.

C₅H₉N₅ (139.16): ′H-NMR (300 MHz, DMSO-d6): mixture of rotamers 6 (ppm)=8.11, 7.

### EXAMPLE 3

Synthesis of Compound 8 in Scheme 3

A mixture of 4-chloro-6,7-dimethoxyquinazoline (500 mg, 2.2 mmol) and methylamine (3 ml, 24 mmol, 8 M in EtOH) was stirred at 100° C. in a sealed vessel until complete conversion of starting material. The reaction mixture was diluted with ethyl acetate, washed with 1 M aq Na₂CO₃, dried over Na₂SO₄, filtered and concentrated. The crude residue was washed with Et₂0 to afford compound 8 as a white solid.

C11H13N302 (219.24): ¹H-NMR (300 MHz, DMSO-d6): 6 (ppm)=8.34 (s, 1H), 7.90 (q, J=4.9, 1H), 7.55 (s, 1H), 7.08 (s, 1H), 3.89 (s, 3H), 3.88 (s, 3H), 3.15 (d, J=4.9, 3H).

### EXAMPLE 4

Synthesis of Compound 9 in Scheme 3

A mixture of methyl-4-chloropicolinate (300 mg, 1.75 mmol) and methylamine (5 ml, 40 mmol, 8 M in EtOH) was stirred at 140° C. in a sealed vessel until complete conversion of starting material. The reaction mixture was diluted with THF and ethyl acetate, washed with 1 M aq Na₂CO₃, dried over Na₂SO₄, filtered and concentrated. The crude residue was adsorbed onto Si0₂ and purified by chromatography (ethyl acetate/EtOH gradient) yielding compound 9 as a white solid.

C8H₁₁N30 (165.19): ¹H-NMR (300 MHz, DMSO-d6): 6 (ppm)=8.53 (q, J=4.9, 1H), 8.06 (d, J=5.6, 1H), 7.17 (d, J=2.4, 1H), 6.82 (q, J=4.9, 1H), 6.55 (dd, J=2.4, 5.6, 1H), 2.75 (d, J=4.9, 3H), 2.73 (d, J=4.9, 3H).

### EXAMPLE 5

Synthesis of Compound 10 in Scheme 3

A mixture of 6-chloro-7-deazapurine (500 mg, 3.24 mmol) and methylamine (3 ml, 24 mmol, 8 M in EtOH) was stirred at 100° C. in a sealed vessel until complete conversion of starting material. The reaction mixture was diluted with THF and ethyl acetate, washed with 1 M aq Na₂CO₃, dried over Na₂SO₄, filtered and concentrated yielding compound 10 as a white solid.

C7H₈N (148.17): ¹H-NMR (300 MHz, DMSO-d₆): 8 (ppm)=11.45 (s, 1H), 8.11 (s, 1H), 7.33 (q, J=4.7, 1H), 7.04 (d, J=3.4, 1H), 6.50 (d, J=3.4, 1H), 2.95 (q, J=4.7, 3H).

### EXAMPLE 6

Synthesis of Compound 12 in Scheme 3

3-Amino-4-methylbenzoic acid (49 g, 0.324 mol) was dissolved in DMF (200 ml) and K₂CO₃ (53.76 g, 0.389 mol) was added. After stirring for 1 h at rt, the suspension was cooled to 0° C. and benzylbromide (42.3 ml, 0.356 mol) was added dropwise over a period of 30 min. The reaction mixture was allowed to warm to rt and stirred for 16 h. To the mixture was added Na₂CO₃ (1 M, aq.). the mixture was extracted with ethyl acetate (3×) and dried (MgS0₄). Evaporation and purification by column chromatography (Si0₂, hexane/ethyl acetate 3:1, 2:1) yielded compound 12 as a pink solid.

Cl₅H₁₅N02 (241.3): TLC (hexane/EtOAc 2:1) R_{f}: 0.55. MS-APCI: 242 ([M+H]⁺). ¹H-NMR (300 MHz, DMSO-d6): 6 (ppm)=7.48-7.31 (m, 5H), 7.29 (d, J=1.7, 1H), 7.13 (dd, J=1.7, J=7.7, 2H), 7.04 (d, J=7.7, 2H), 5.29 (s, 2H), 5.13 (bs, 1H), 2.11 (s, 3H).

### EXAMPLE 7

Synthesis of Compound 13 in Scheme 3

Compound 12 (Example 6; 16.95 g, 70.24 mmol) was dissolved in CH₂CI₂ (150 ml) and cooled to 0° C. To the solution was added phosgene solution (73.9 ml, 140.49 mmol, 20% in toluene) within 1 Os and stirred for 1 min. To the reaction mixture was added cold saturated aqueous NaHCO₃ solution (300 ml) and it was stirred vigorously for 15 min at 0° C. To the reaction mixture was added saturated aqueous NaCl and the organic layer was separated. The aqueous phase was exctracted with CH₂CI₂ and both combined organic layers were dried (MgS0₄), and concentrated (at 25° C. bath temp.) to yield compound 13 as light brown oil.

C16H13N03 (267.3): ¹H-NMR (300 MHz, DMSO-d6): 6 (ppm)=7.76 (m, 2H), 7.50-7.30 (m, 6H), 5.34 (s, 2H), 2.36 (s, 3H).

### EXAMPLE 8

Synthesis of Ureas 14 in Scheme 3

A mixture of isocyanate 13 (scheme 1; 1.05 eq) and methyl aryl amine 6 (or 7) (1 eq) in dry dioxane was heated at reflux in a sealed vessel under a nitrogen atmosphere until completion of the reaction. After cooling to rt, the mixture was filtrated and the precipitate was washed with ethyl acetate yielding Urea 14 as a white solid.

C22H23N503 (405.45): ¹H-NMR (300 MHz, DMSO-d6): 6 (ppm)=13.10 (s, 1H), 8.74 (d, J=1.5, 1H), 8.32 (s, 1H), 7.60 (dd, J=1.7, 7.7, 1H), 7.51-7.31 (m, 7H), 6.04 (s, 1H), 5.35 (s, 2H), 3.31 (s, 3H), 2.84 (d, J=4.5, 3H), 2.39 (s, 3H).

### EXAMPLE 9

Synthesis of Ureas 15 in Scheme 3

The title compound was made using a method analogous to that described in Example 8. C21H22N603 (406.18): ¹H-NMR (300 MHz, DMSO-d6): mixture of rotamers 6 (ppm)=12.77, 12.57 (s, 1H), 8.66, 8.54 (d, J=1.6, 1H), 8.51, 8.39 (s, 1H), 8.18, 8.06 (d, J=4.2, 1H), 7.69-7.63 (m, 1H), 7.49-7.34 (m, 6H), 5.35 (s, 2H), 3.31 (s, 3H), 3.38, 3.44 (s, 3H), 2.86, 2.85 (d, J=4.2, 3H), 2.39 (s, 3H).

### EXAMPLE 10

Synthesis of Ureas 17 in Scheme 3

A mixture of isocyanate 13 (870 mg, 3.25 mmol) and methylamine (500 g, 3 mmol in EtOH free CHCI₃ (1.5 ml) was heated in a microwave apparatus at 175° C. for 80 min. after cooling, the mixture was concentrated and the crude residue was adsorbed on Si0₂ and purified by chromatography (CH₂CI₂/EtOH gradient) yielding urea 17 as a brown foam. C24H24N404 (432.47): ¹H-NMR (300 MHz, DMSO-d₆): 6 (ppm)-8.82 (s, 1H), 8.71 (d, J=4.9, 1H), 8.49 (d, J=5.6, 1H), 8.02 (d, J=2.4, 1H), 7.92 (d, J=1.3, 1H), 7.73 (dd, J=1.3, 7.9, 1H), 7.56 (dd, J=2.4, 5.7, 1H), 7.47-7.38 (m, 6H), 5.35 (s, 2H), 3.43 (s, 3H), 2.81 (d, J=4.9, 3H), 2.30 (s, 3H).

### EXAMPLE 11

Synthesis of Acid 19 in Scheme 3

A mixture of urea 14 (or 15) (1 eq) in 2-methoxyethanol/dioxane (8/2) and Pd/C 10% (0.08 eq) was stirred at 80° C. under H₂ atmosphere until completion of the reaction. The reaction mixture was filtrated and concentrated to yield the title compound as a white solid. C₁₅H₁₇N₅03 (315.33): ¹H-NMR (300 MHz, DMSO-d₆): 6 (ppm)=13.02 (s, 1H), 8.65 (d, J=1.4, 1H), 8.33 (s, 1H), 7.55 (dd, J=1.7, 7.9, 1H), 7.47 (q, J=4.7, 1H), 7.31 (d, J=8.0, 1H), 6.06 (s, 1H), 3.25 (s, 3H), 2.84 (d, J=4.7, 3H), 2.38 (s, 3H).

### EXAMPLE 12

Synthesis of Acid 20 in Scheme 3

The title compound was made using a method analogous to that described in Example 11. C14H16N603 (316.32): ¹H-NMR (300 MHz, DMSO-d₆): mixture of rotamers 6 (ppm)=12.80 (brs, 1H), 12.72, 12.48 (s, 1H), 8.60, 8.48 (d, J=1.4, 1H), 8.50, 8.38 (s, 1H), 8.19, 8.09 (q, J=4.1, 1H), 7.64-7.57 (m, 1H), 7.38-7.32 (m, 1H), 3.44, 3.38 (s, 3H), 2.86, 2.85 (d, J=4.1, 3H), 2.36 (s, 3H).

### EXAMPLE 13

Synthesis of Acid 22 in Scheme 3

A mixture of urea 17 (3.5 g, 8.09 mmol) in MeOH (130 ml) and Pd/C 10% (350 mg, 10% m) was stirred at rt under an H₂ atmosphere until completion of the reaction. The reaction was filtered and concentrated under vacuo. The crude residue was washed with Et₂0 yielding Acid 22 as a yellow solid. C₁₇H₁₈N₄O₄ (342.35): ¹H-NMR (300 MHz, DMSO-d6): 6 (ppm)=8.78 (s, 1H), 8.71 (q, J=4.9, 1H), 8.49 (d, J=5.6, 1H), 8.02 (d, J=2.3, 1H), 7.88 (d, J=1.35, 1H), 7.67 (dd, J=1.6, 7.8, 1H), 7.57 (dd, J=2.3, 5.6, 1H), 7.32 (d, J=7.9, 1H), 3.44 (s, 3H), 2.81 (d, J=4.9, 3H), 2.29 (s, 3H).

### EXAMPLE 14

General Procedure for the Synthesis of Amides Using the HATU/HOAt Coupling Acid 19 (20 or 22) in Scheme 3

(1 eq), HATU (1.5 eq), HOAt (1.5 eq), amine (1 eq) and i-Pr₂EtN (3 eq) in DMF were combined in a sealed vessel and stirred at rt or 85° C. until completion of the reaction. The reaction mixture was diluted with ethyl acetate and washed with saturated aqueous Na₂CO₃ and brine, dried over Na₂SO₄, filtered and concentrated. The crude residue was washed with ethyl acetate, EtOH or THF and/or purified by chromatography on silica gel to afford the title compound amide 31 of scheme 4.

Example: R⁴═H and R⁵=3-methoxy-5-trifluoromethylaniline C23H23N603 (488.46): TLC (AcOEt/EtOH 1:1) R_{f}: 0.44. MS-APCI: 489 ([M+H]⁺). Analyt. HPLC (system A) R_{T} in min (purity)=4.95 (97) ′H-NMR (300 MHz, DMSO-d₆): 6 (ppm)=13.05 (s, 1H), 10.40 (s, 1H), 8.60 (s, 1H), 8.35 (s, 1H), 7.84 (s, 1H), 7.74 (s, 1H), 7.61 (dd, J=1.5, 7.8, 1H), 7.47 (q, J=4.2, 1H), 7.39 (d, J=7.9, 1H), 6.96 (s, 1H), 6.07 (s, 1H), 3.84 (s, 3H), 3.30 (s, 3H), 2.84 (d, J=4.2, 3H), 2.40 (s, 3H).

### EXAMPLE 15

General Procedure for the Synthesis of Amides Using Acid Chloride to Couple Acid 19 (20 or 22) in Scheme 3

To a mixture of acid 19 (20 or 22) (1 eq) in EtOH (CHCI₃ free) was added oxalyl chloride (1.9 eq). After 5 min, DMF (8.3 eq) was added dropwise at 0° C. and stirring was continued for 60-90 min. The mixture was poured into an ice cold CH₃CI solution of aniline (3.5 eq), and stirring was continued for 16 h at rt or 55° C. the reaction mixture was diluted with ethyl acetate, washed with saturated aqueous Na₂CO₃ and brine, dried over Na₂SO₄, filtered and concentrated. The crude residue was washed with ethyl acetate, EtOH or THF and/or purified by chromatography on silica gel to afford the title compound amide 31 of scheme 4.

Example: R⁴═H and R⁵=3-trifluoromethylaniline

Calc. For C24H22F3N503=485.46; MS-ESI: found 486 ([M+H]⁺). Analyt. HPLC (system A) RT in min (purity)=4.16 (99%); TLC (AcOEt/hexane 8:2) Rf: 0.21.

The following Exemplary compounds were synthesized using a method analogous to one or more of those described in Examples 1-15.

| Ex. | | | Mol. | MS |
|---|---|---|---|---|
| No. | Structure | Structure Name | Form | Data |
| 16 | 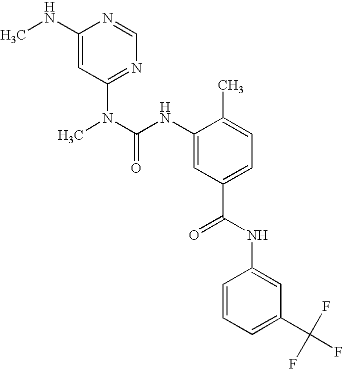 | ′4-methyl-3- (((methyl(6- (methylamino)- 4-pyrimidinyl) amino)carbonyl) amino)-N-(3- (trifluoromethyl) phenyl) benzamide | C22H21F3N6O2 | 458 |
| 17 | 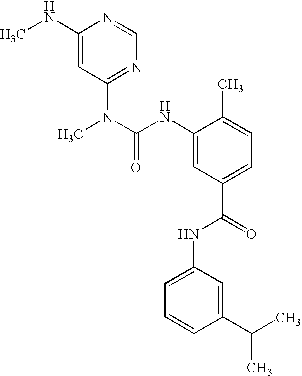 | 4-methyl-N-(3- (1-methylethyl) phenyl)-3- (((methyl(6- (methylamino)- 4-pyrimidinyl) amino) carbonyl) amino) benzamide | C24H28N6O2 | 432 |
| 18 | 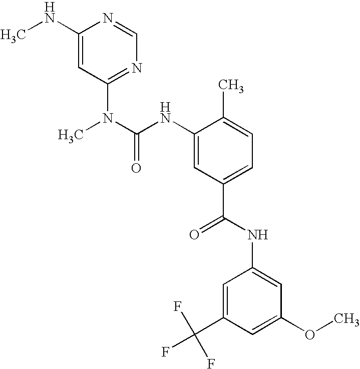 | 4-methyl-3- (((methyl(6- (methylamino)- 4-pyrimidinyl) amino)carbonyl) amino)-N-(3- (methyloxy)-5- (trifluoromethyl) phenyl) benzamide | C23H23F3N6O3 | 488 |
| 19 | 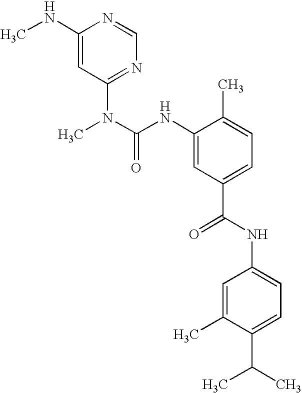 | 4-methyl-3- (((methyl(6- (methylamino)- 4-pyrimidinyl) amino)carbonyl amino)-N-(3- methyl-4-(1- methylethyl) phenyl)benzamide | C25H30N6O2 | 447 |
| 20 | 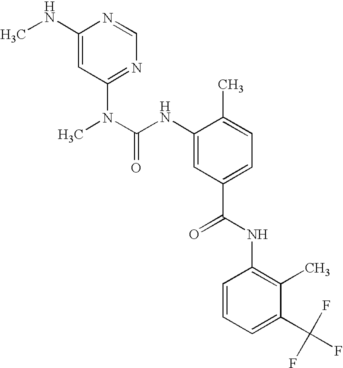 | 4-methyl-3- (((methyl(6- (methylamino)- 4-pyrimidinyl) amino)carbonyl) amino)-N-(2- methyl-3- (trifluoromethyl) phenyl)benzamide | C23H23F3N6O2 | 472 |
| 21 | 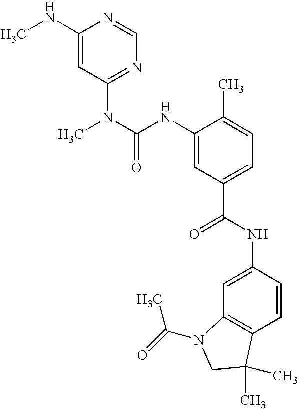 | N-(1-acetyl- 3,3-dimethyl- 2,3-dihydro- 1H-indol-6- yl)-4-methyl- 3-(((methyl(6- (methylamino)- 4-pyrimidinyl) amino)carbonyl) amino)benzamide | C27H31N7O3 | 502 |
| 22 | 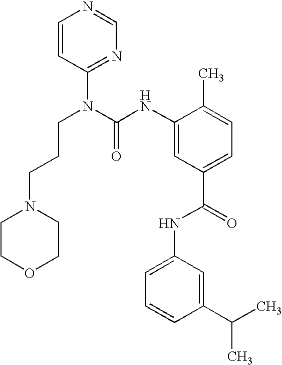 | 4-methyl-N-(3- (1-methylethyl) phenyl)-3-((((3- (4-morpholinyl) propyl)(4- pyrimidinyl) amino)carbonyl) amino)benzamide | C29H36N6O3 | 517 |
| 23 | 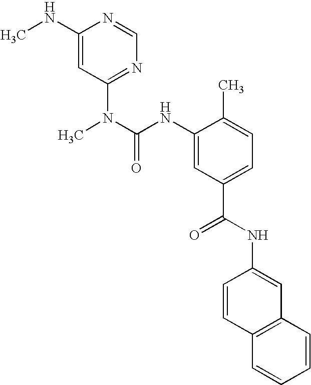 | 4-methyl-3- (((methyl(6- (methylamino)- 4-pyrimidinyl) amino)carbonyl) amino)-N-(2- naphthalenyl) benzamide | C25H24N6O2 | 441 |
| 24 | 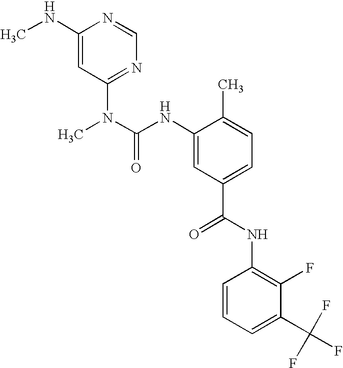 | N-(2-fluoro-3- (trifluoromethyl) phenyl)-4- methyl-3- ((methyl(6- (methylamino)- 4-pyrimidinyl) amino)carbonyl amino)benzamide | C22H20F4N6O2 | 476 |
| 25 | 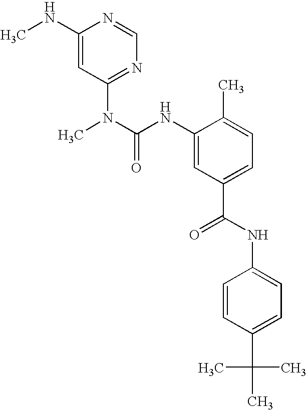 | N-(4-1,1- dimethylethyl) phenyl)-4- methyl-3- ((methyl(6- (methylamino)- 4-pyrimidinyl) amino)carbonyl amino)benzamide | C25H30N6O2 | 447 |
| 26 | 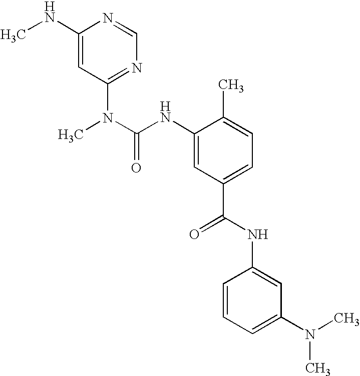 | N-(3- (dimethylamino) phenyl)-4- methyl-3- ((methyl(6- (methylamino)- 4-pyrimidinyl) amino)carbonyl amino)benzamide | C23H27N7O2 | 434 |
| 27 | 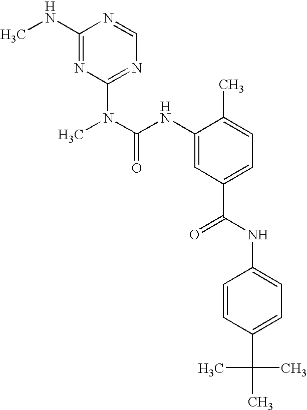 | N-(4-1,1- (dimethylethyl) phenyl)-4- methyl-3- (((methyl(4- (methylamino)- 1,3,5-triazin-2- yl)amino)carbonyl) amino)benzamide | C24H29N7O2 | 448 |
| 28 | 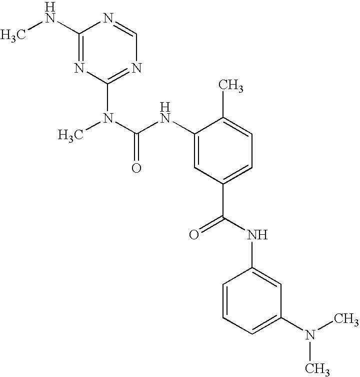 | N-(3- (dimethylamino) phenyl)-4- methyl-3- (((methyl-4- (methylamino)- 1,3,5-triazin-2- yl)amino)carbonyl) amino)benzamide | C22H26N8O2 | 435 |
| 29 | 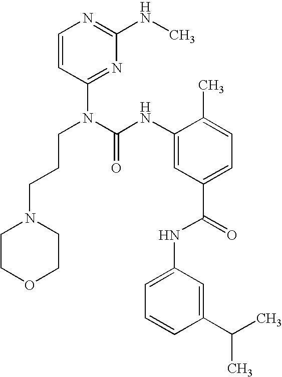 | 4-methyl-3- ((((2- (methylamino)- 4-pyrimidinyl) (3-(4- morpholinyl) propyl)amino) carbonyl)amino)- N-(3-(1- methylethyl) phenyl)benzamide | C30H39N7O3 | 546 |
| 30 | 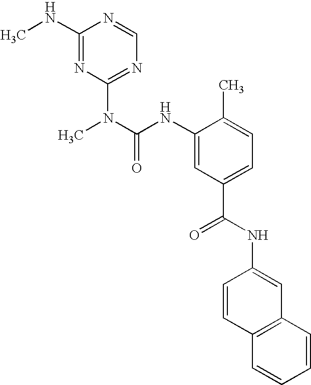 | 4-methyl-3- (((methyl(4- (methylamino)- 1,3,5-triazin-2- yl)amino)carbonyl) amino)-N-(2- naphthalenyl) benzamide | C24H23N7O2 | 441 |
| 31 | 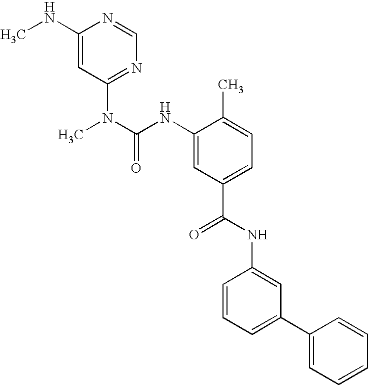 | N-(1,1′- biphenyl-3- yl)-4-methyl- 3-(((methyl(6- (methylamino)- 4-pyrimidinyl) amino)carbonyl) amino)benzamide | C27H26N6O2 | 467 |
| 32 | 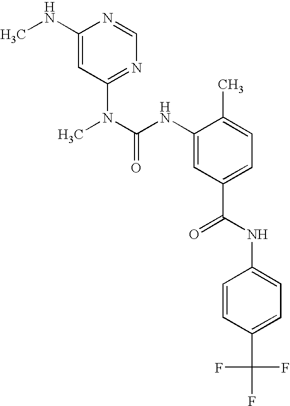 | 4-methyl-3- (((methyl(6- (methylamino)- 4-pyrimidinyl) amino)carbonyl) amino)-N-(4- (trifluoromethyl) phenyl)benzamide | C22H21F3N6O2 | 458 |
| 33 | 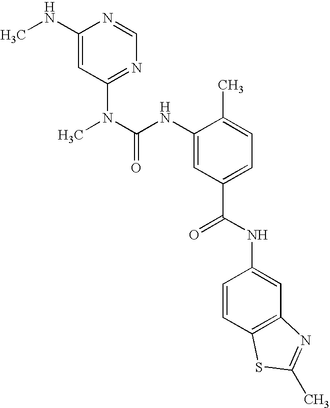 | 4-methyl-N-(2- methyl-1,3- benzothiazol- 5-yl)-3- (((methyl(6- (methylamino)- 4-pyrimidinyl) amino)carbonyl) amino)benzamide | C23H23N7O2S | 462 |
| 34 | 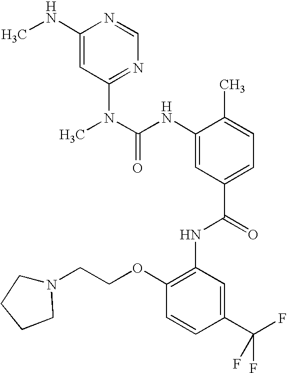 | 4-methyl-3- (((methyl(6- (methylamino)- 4-pyrimidinyl) amino)carbonyl) amino)-N-(2- ((2-(1- pyrrolidinyl) ethyl)oxy)-5- (trifluoromethyl) phenyl)benzamide | C28H32F3N7O3 | 572 |
| 35 | 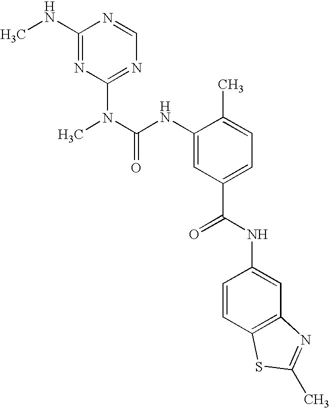 | 4-methyl-N-(2- methyl-1,3- benzothiazol- 5-yl)-3- (((methyl(4- (methylamino)- 1,3,5-triazin-2- yl)amino)carbonyl) amino)benzamide | C22H22N8O2S | 463 |
| 36 | 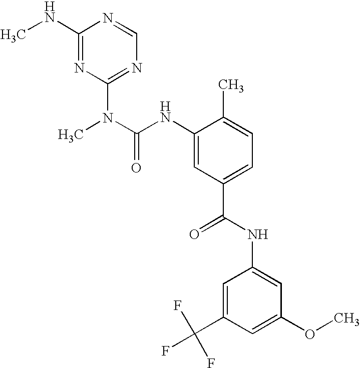 | 4-methyl-3- (((methyl(4- (methylamino)- 1,3,5-triazin-2- yl)amino)carbonyl) amino)-N-(3- (methyloxy)-5- (trifluoromethyl) phenyl)benzamide | C22H22F3N7O3 | 489 |
| 37 | 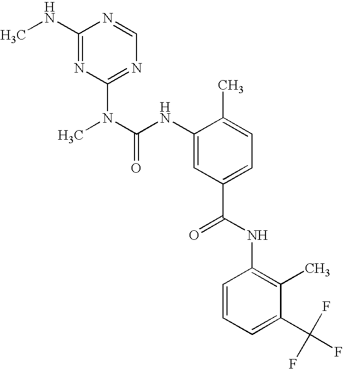 | 4-methyl-3- (((methyl(4- (methylamino)- 1,3,5-triazin-2- yl)amino)carbonyl) amino)-N-(2-methyl-3- (trifluoromethyl) phenyl)benzamide | C22H22F3N7O2 | 473 |
| 38 | 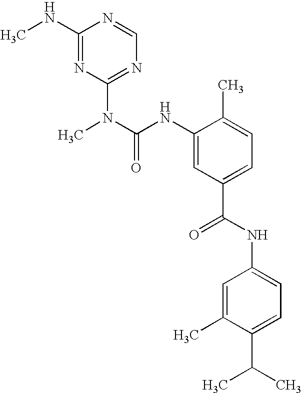 | 4-methyl-3- (((methyl(4- (methylamino)- 1,3,5-triazin-2- yl)amino)carbonyl) amino)-N- (3-methyl-4-(1- methylethyl) phenyl)benzamide | C24H29N7O2 | 448 |
| 39 | 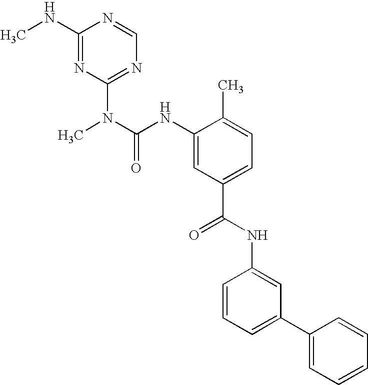 | N-(1,1′- biphenyl-3- yl)-4-methyl- 3-(((methyl(4- (methylamino)- 1,3,5-triazin-2- yl)amino)carbonyl) amino)benzamide | C26H25N7O2 | 468 |
| 40 | 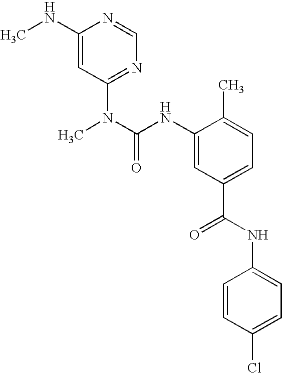 | N-(4- chlorophenyl)- 4-methyl-3- (((methyl(6- (methylamino)- 4-pyrimidinyl) amino)carbonyl) amino)benzamide | C21H21ClN6O2 | 425 |
| 41 | 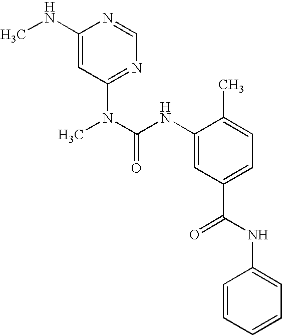 | 4-methyl-3- (((methyl(6- (methylamino)- 4-pyrimidinyl) amino)carbonyl) amino)-N- phenylbenzamide | C21H22N6O2 | 390 |
| 42 | 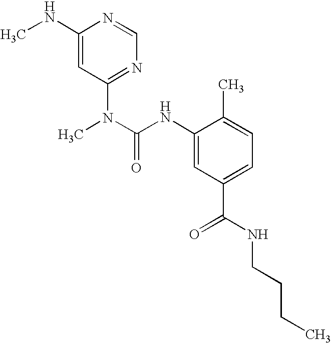 | N-butyl-4- methyl-3- (((methyl(6- (methylamino)- 4-pyrimidinyl) amino)carbonyl) amino)benzamide | C19H26N6O2 | 370 |
| 43 | 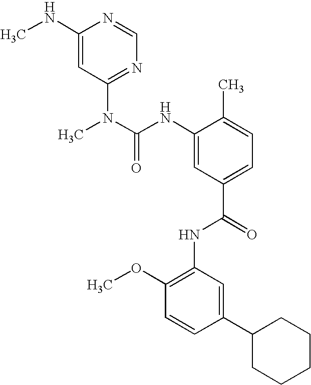 | N-(5- cyclohexyl-2- (methyloxy) phenyl)-4-methyl-3- (((methyl(6- (methylamino)- 4-pyrimidinyl) amino)carbonyl) amino)benzamide | C28H34N6O3 | 503 |
| 44 | 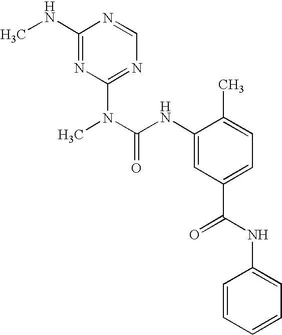 | 4-methyl-3- (((methyl(4- (methylamino)- 1,3,5-triazon-2- yl)amino)carbonyl) amino)-N- phenylbenzamide | C20H21N7O2 | 391 |
| 45 | 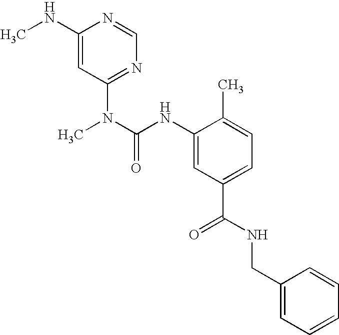 | 4-methyl-3- (((methyl(6- (methylamino)- 4-pyrimidinyl) amino)carbonyl) amino)-N- (phenylmethyl) benzamide | C22H24N6O2 | 404 |
| 46 | 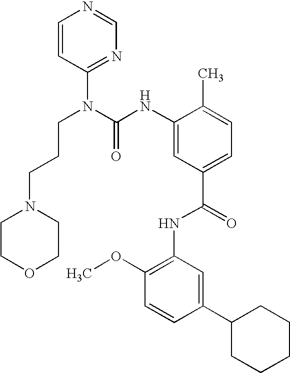 | N-(5- cyclohexyl-2- (methyloxy) phenyl)-4-methyl- 3-((((3-(4- morpholinyl) propyl)(4- pyrimidinyl) amino)carbonyl) amino)benzamide | C33H42N6O4 | 587 |
| 47 | 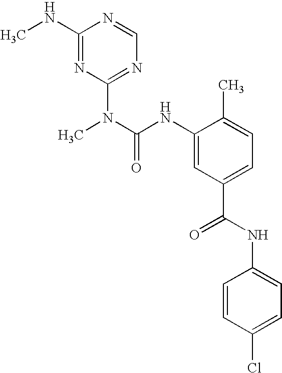 | N-(4- chlorophenyl)- 4-methyl-3- (((methyl(4- (methylamino)- 1,3,5-triazin-2- yl)amino) carbonyl)amino) benzamide | C20H20ClN7O2 | 426 |
| 48 | 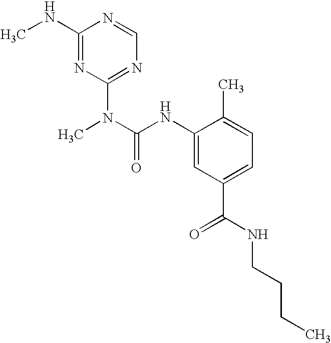 | N-butyl-4- methyl-3- (((methyl(4- (methylamino)- 1,3,5-triazin-2- yl)amino)carbonyl) amino)benzamide | C18H25N7O2 | 371 |
| 49 | 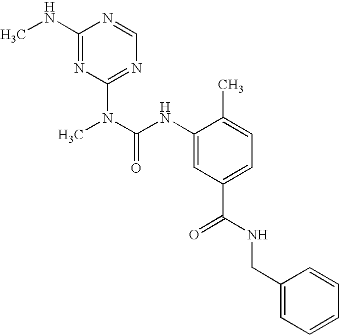 | 4-methyl-3- (((methyl(4- (methylamino)- 1,3,5-triazin-2- yl)amino)carbonyl) amino)-N- (phenylmethyl) benzamide | C21H23N7O2 | 405 |
| 50 | 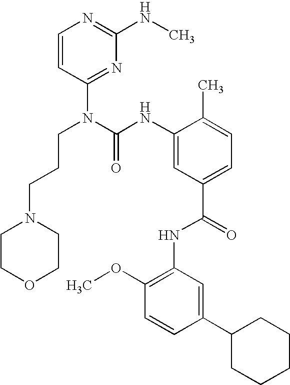 | N-(5- cyclohexyl-2- (methyloxy) phenyl)-4-methyl- 3-((((2- (methylamino)- 4-pyrimidinyl)(3- (4-morpholinyl) propyl)amino) carbonyl)amino) benzamide | C34H45N7O4 | 616 |
| 51 | 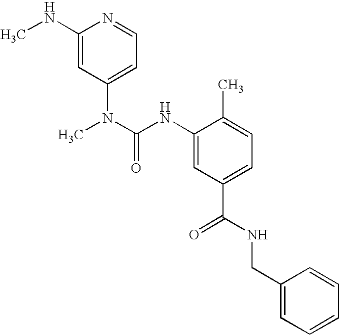 | N-methyl-4- (methyl(((2- methyl-5- (((phenylmethyl) amino)carbonyl) phenyl)amino) carbonyl)amino)-2- pyridinecarboxamide | C24H25N5O3 | 431 |
| 52 | 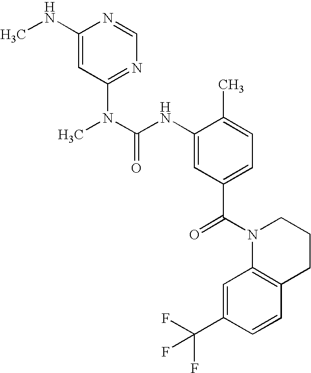 | N-methyl-N-(6- (methylamino)- 4-pyrimidinyl)- N′-(2-methyl- 5-((7- (trifluoromethyl)- 3,4-dihydro-1(2H)- quinolinyl) carbonyl)phenyl) urea | C25H25F3N6O2 | 499 |
| 53 | 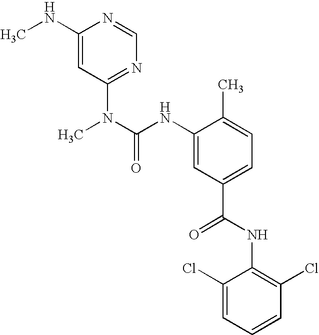 | N-(2,6- dichlorophenyl)- 4-methyl-3- (((methyl(6- (methylamino)- 4-pyrimidinyl) amino)carbonyl) amino)benzamide | C21H20Cl2N6O2 | 459 |
| 54 | 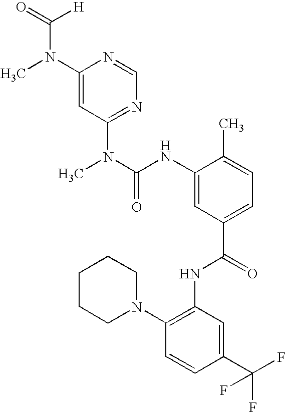 | 3-((((6- (formyl(methyl) amino-4- pyrimidinyl) (methyl)amino) carbonyl)amino)- 4-methyl-N-(2- (1-piperidinyl)-5- (trifluoromethyl) phenyl)benzamide) | C28H30F3N7O3 | 570 |
| 55 | 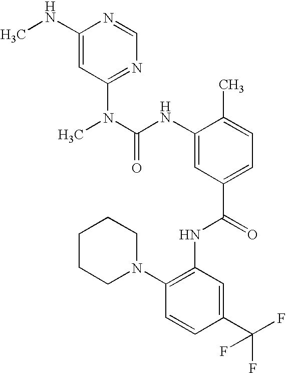 | 4-methyl-3- (((methyl(6- (methylamino)-4- pyrimidinyl) amino)carbonyl) amino)-N-(2-(1- piperidinyl)-5- (trifluoromethyl) phenyl)benzamide | C27H30F3N7O2 | 542 |
| 56 | 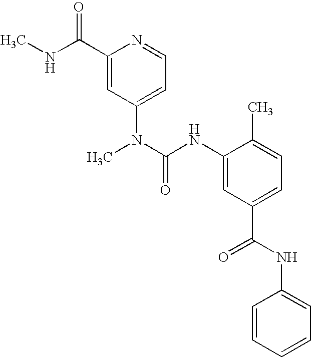 | N-methyl-4- (methyl(((2- methyl-5- ((phenylamino) carbonyl)phenyl) amino)carbonyl) amino)-2- pyridinecarboxamide | C23H23N5O3 | 417 |
| 57 | 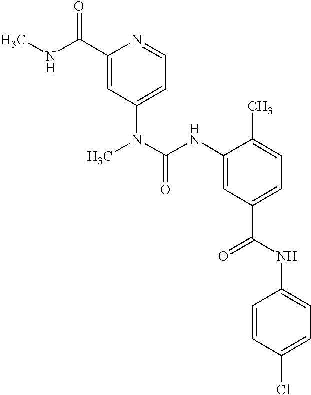 | 4-((((5-(((4- chlorophenyl) amino)carbonyl)- 2-methylphenyl) amino)carbonyl) (methyl)amino)- N-methyl-2- pyridinecarboxamide | C23H22ClN5O3 | 452 |
| 58 | 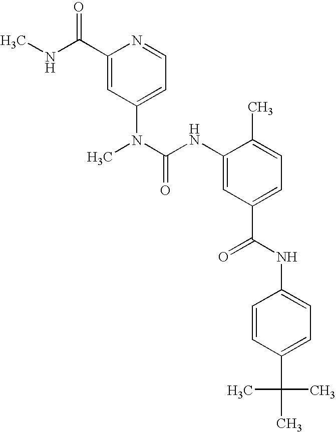 | 4-((((5-(((4-(1,1- dimethylethyl) phenyl)amino) carbonyl)-2- methylphenyl) amino)carbonyl) (methyl)amino)- N-methyl-2- pyridinecarboxamide | C27H31N5O3 | 474 |
| 59 | 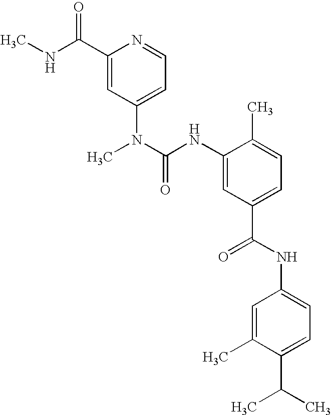 | N-methyl-4- (methyl(((2- methyl-5-(((3- methyl-4-(1- methylethyl) phenyl)amino) carbonyl)phenyl) amino)carbonyl) amino)-2- pyridinecarboxamide | C27H31N5O3 | 474 |
| 60 | 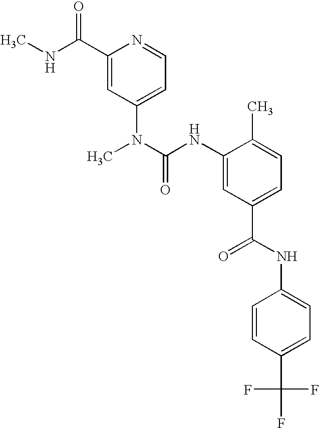 | N-methyl-4- (methyl(((2- methyl-5-(((4- (trifluoromethyl) phenyl)amino carbonyl)phenyl) amino)carbonyl amino)-2- pyridinecarboxamide | C24H22F3N5O3 | 485 |
| 61 | 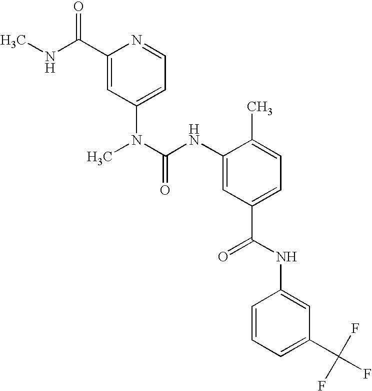 | N-methyl-4- (methyl(((2- methyl-5-(((3- (trifluoromethyl) phenyl)amino) carbonyl)phenyl) amino)carbonyl) amino)-2- pyridinecarboxamide | C24H22F3N5O3 | 485 |
| 62 | 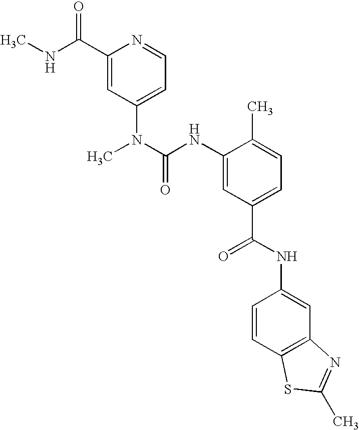 | N-methyl-4- (methyl(((2- methyl-5-(((2- methyl-1,3- benzothiazol-5- yl)amino)carbonyl) phenyl)amino) carbonyl)amino)-2- pyridinecarboxamide | C25H24N6O3S | 489 |
| 63 | 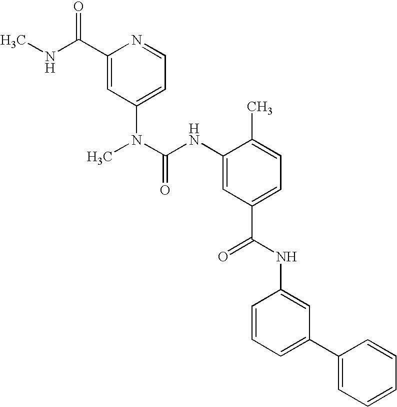 | 4-((((5-((1,1′- biphenyl-3- ylamino)carbonyl)-2- methylphenyl) amino)carbonyl) (methyl)amino)- N-methyl-2- pyridinecarboxamide | C29H27N5O3 | 494 |
| 64 | 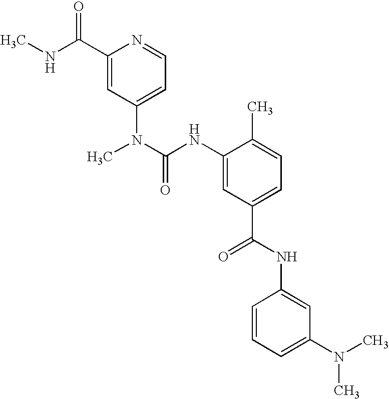 | 4-((((5-(((3- (dimethylamino) phenyl)amino) carbonyl)-2- methylphenyl) amino)carbonyl) (methyl)amino)- N-methyl-2- pyridinecarboxamide | C25H28N6O3 | 461 |
| 65 | 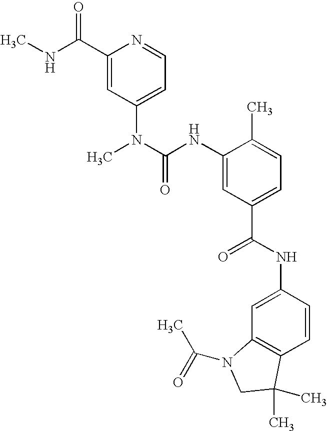 | 4-((((5-(((1- acetyl-3,3- dimethyl-2,3- dihydro-1H- indol-6- yl)amino) carbonyl)-2- methylphenyl) amino)carbonyl) (methyl)amino)- N-methyl-2- pyridinecarboxamide | C29H32N6O4 | 529 |
| 66 | 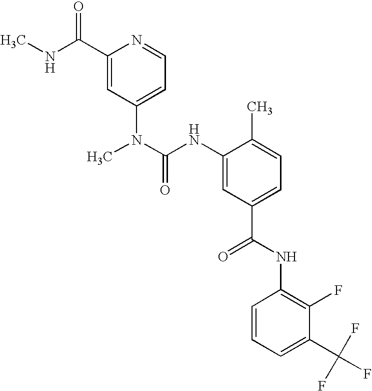 | 4-((((5-(((2- fluoro-3- (trifluoromethyl) phenyl)amino) carbonyl)-2- methylphenyl) amino)carbonyl) (methyl)amino)- N-methyl-2- pyridinecarboxamide | C24H21F4N5O3 | 503 |
| 67 | 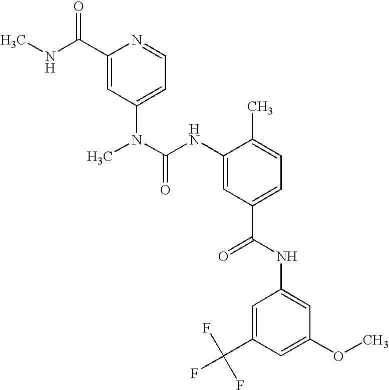 | N-methyl-4- (methyl(((2- methyl-5-(((3- (methyloxy)-5- (trifluoromethyl) phenyl)amino) carbonyl)phenyl) amino)carbonyl) amino)-2- pyridinecarboxamide | C25H24F3N5O4 | 515 |
| 68 | 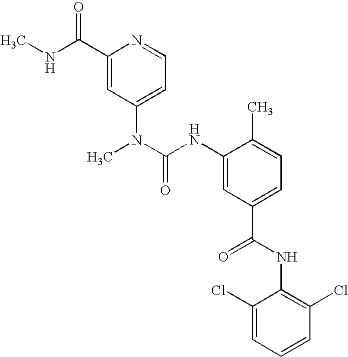 | 4-((((5-(((2,6- dichlorophenyl) amino)carbonyl)-2- methylphenyl) amino)carbonyl (methyl)amino)- N-methyl-2- pyridinecarboxamide | C23H21Cl2N5O3 | 486 |
| 69 | 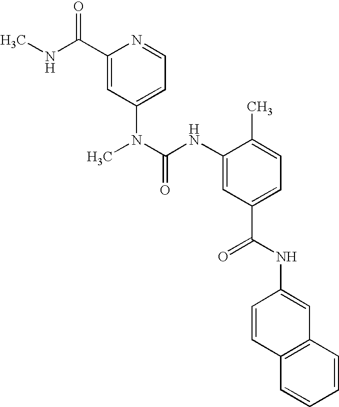 | N-methyl-4- (methyl(((2- methyl-5-((2- naphthalenylamino) carbonyl)phenyl) amino)carbonyl) amino)-2- pyridinecarboxamide | C27H25N5O3 | 468 |
| 70 | 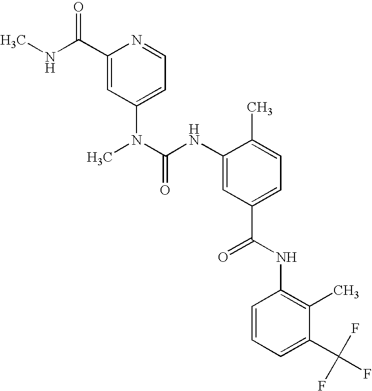 | N-methyl-4- (methyl(((2- methyl-5-(((2- methyl-3- (trifluoromethyl) phenyl)amino) carbonyl)phenyl) amino)carbonyl) amino)-2- pyridinecarboxamide | C25H24F3N5O3 | 499 |
| 71 | 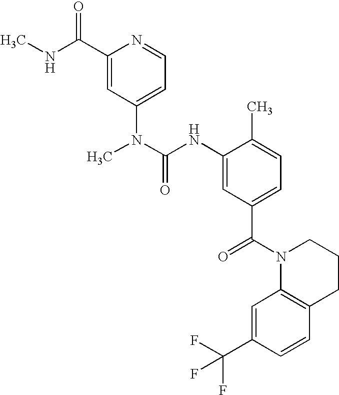 | N-methyl-4- (methyl(((2- methyl-5-((7- (trifluoromethyl)-3,4- dihydro-1(2H)- quinolinyl) carbonyl)phenyl) amino)carbonyl) amino)-2- pyridinecarboxamide | C27H26F3N5O3 | 526 |

The following additional Example 72, including starting reagents and intermediates, is set forth to further enhance the understanding and appreciation of the present invention.

### EXAMPLE 72

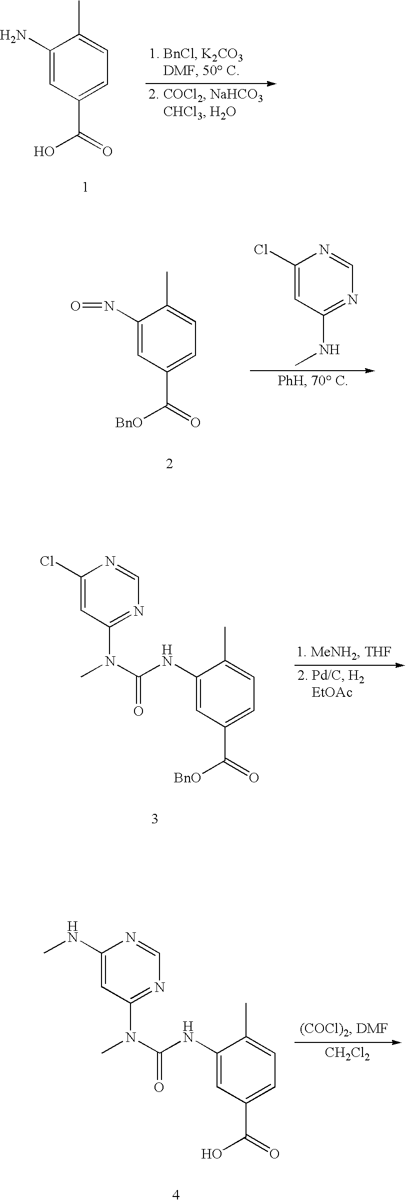 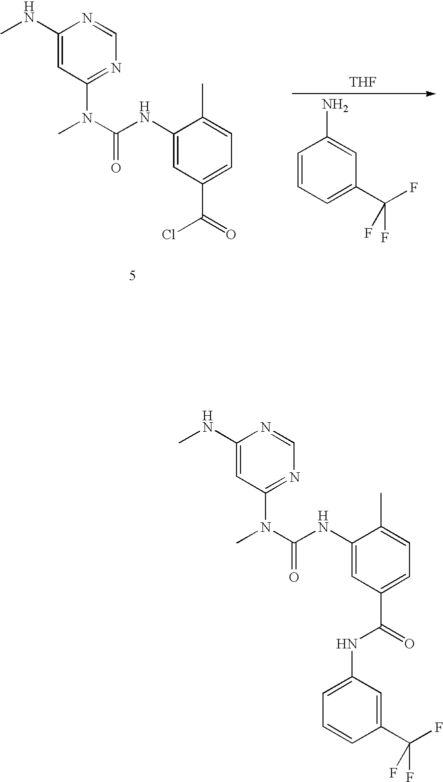 Step 1:

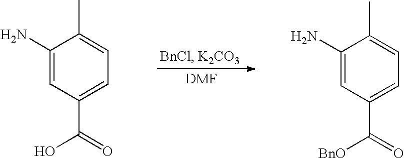

To the amino acid 1 (10.00 g, 66.15 mmol) in DMF (50 mL) at room temperature was added K₂CO₃ (10.97 g, 79.38 mmol). The reaction was stirred vigorously for 10 minutes followed by the addition of benzyl chloride (8.37 mL, 72.77 mmol). The reaction was then heated at 50° C. until consumption of starting material as indicated by TLC was complete. The mixture was diluted with 500 ml H₂O and extracted with EtOAc (3×100 mL). The EtOAc was then washed with H₂O and brine, followed by drying with MgSO₄. Filtration and removal of solvent under reduced pressure furnished a red/purple solid. Trituration with Et₂O removed most of the colored material (high R_{f}), leaving the desired product as a light-pink solid. Chromatography on silica gel afforded pure benzyl ester.
Step 2:

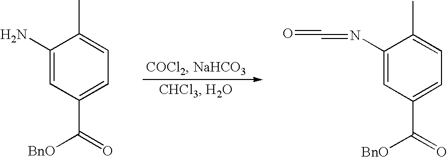

To the aniline (5.00 g, 20.72 mmol) in CHCl₃ (150 mL) at room temperature was added NaHCO₃ (aq., sat. 150 mL). The reaction was stirred vigorously for 10 minutes before allowing the layers to separate. Phosgene (20%, 16.44 mL, 31.08 mmol) was syringed into the lower, organic layer and the reaction was again stirred vigorously for 10 minutes. The layers were then separated and the aqueous extracted with CH₂Cl₂ (2×50 mL). The organics were combined, washed with brine, dried with MgSO₄ and filtered. Removal of solvent provided nearly pure isocyanate 2 as a colorless oil (waxy solid after standing in freezer).
Step 3:

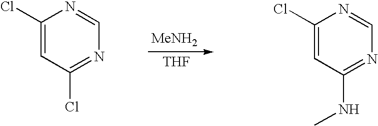

To 4.6-dichloropyrimidine in THF at 0° C. was added MeNH₂ (1.5 equiv.) dropwise (slight exotherm). The reaction was allowed to warm to room temperature and stirred for an additional 6 hours. Solvent was removed under reduced pressure and the reaction mixture was taken up in EtOAc and washed twice with NaHCO₃ (aq., sat.). The organic layer was then washed with brine, dried with MgSO₄ and filtered. Removal of solvent yielded the chloro-amino-pyrimidine product as a white solid.
Step 4:

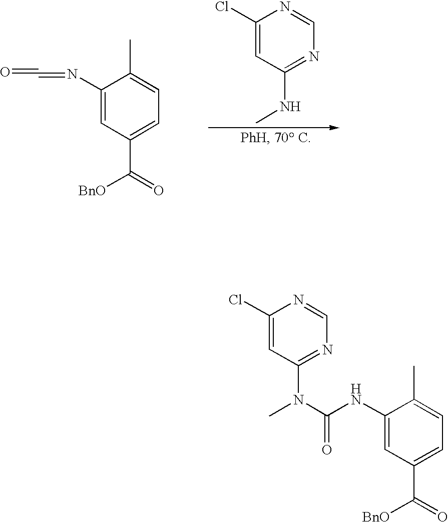

The isocyanate and chloro-amino-pyrimidine (1:1) were combined in benzene and heated at 70° C. for 2 days. A solid precipitate forms. The reaction was cooled to room temperature and filtered, affording the urea 3 as a white solid.
Step 5:

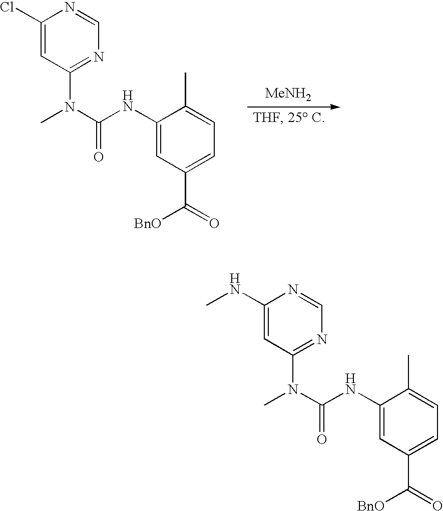

To the aryl chloride 3 was added MeNH₂ in THF (2.0 M, excess) at 25° C. The reaction was stirred at room temperature until completion as indicated by LCMS (usually several hours). Solvent and excess amine was removed under reduced pressure and the crude mixture was dissolved in EtOAc/H₂O. The layers were separated and the aqueous was extracted with additional EtOAc. The organics were combined, washed with brine, dried with MgSO₄ and filtered. Removal of solvent afforded the desired product, which could be purified by silica gel chromatography.
Step 6:

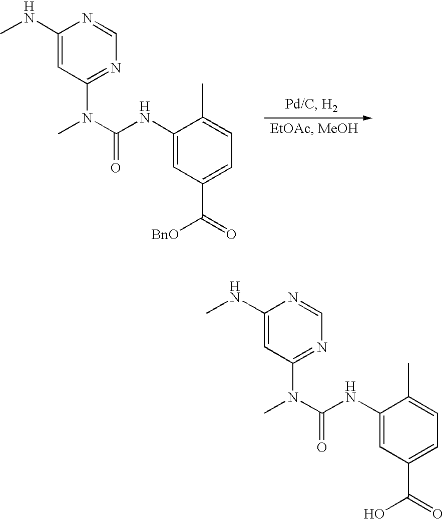

The ester from Step 5 (3.00 g, 7.40 mmol) was suspended in EtOAc/MeOH (10:1, 200 mL) at room temperature. Pd/C (catalytic) was added under nitrogen and the flask carefully purged with H₂ gas. The flask was capped with a rubber septum and positive H₂ gas pressure was applied through a balloon/needle. The reaction was stirred at room temperature overnight (solid precipitate) and then extracted with NaOH (aq., 2N, 3×30 mL). The aqueous extracts were then neutralized by slow addition of HCl (aq., 6N) and a solid precipitates. The mixture was filtered and the solids dissolved in CH₂Cl₂/MeOH (1:1). Filtration removeed remaining catalyst. Removal of solvents under reduced pressure afforded the product acid as a white solid. Product was carried on as a crude mixture.
Step 7:

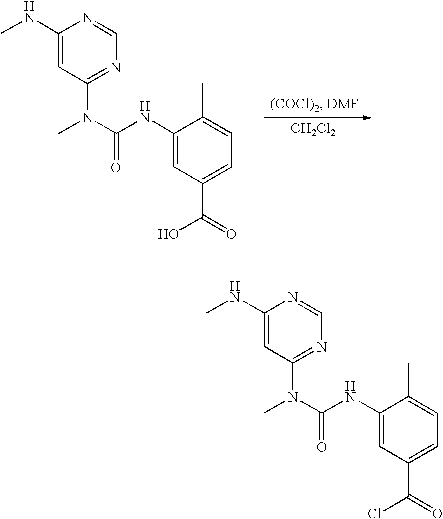

The acid (1.00 g, 3.17 mmol) was suspended in CH₂Cl₂ (30 mL) at room temperature and DMF (1 drop, catalytic) was added. Oxalyl chloride was then added and the reaction stirred at room temperature for several hours (the reaction progress was monitored by quenching of small aliquots with MeOH or a nucleophilic amine followed by LCMS analysis). The solvent was removed under reduced pressure and the product acid chloride 5 used as a crude mixture in step 8.
Step 8:

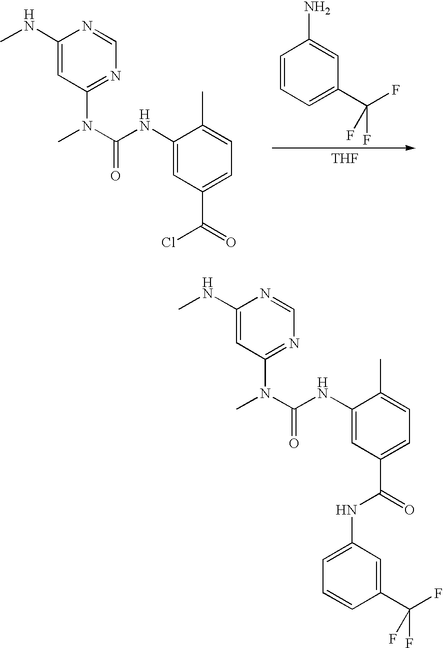

To the acid chloride 5 (100 mg, 0.27 mmol) suspended in THF was added 3-trifluoromethyl aniline (0.037 mL, 0.30 mmol). The reaction was stirred at room temperature for several hours. The solvent was removed and the mixture was taken up in EtOAc (5 mL) and washed with NaOH (aq., 2N, 2 mL). The layers were separated and the aqueous was extracted with EtOAc (5 mL). The organics were combined, washed with brine, dried with MgSO₄ and filtered. After removal of the solvent, chromatography afforded pure amide product 6. MS=found M+H⁺=458.

Note: Products such as compound 6 can be purified by normal phase silica gel chromatography or reverse phase HPLC. Alternatively, in some cases, trituration with MeOH or other solvents may afford pure products.

### EXAMPLE 73

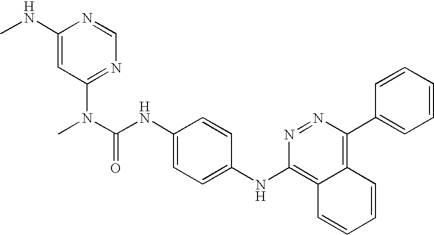

The following are additional exemplary compounds A1-A20, B1-B20, C1-C20, D1-D20 and E1-E20, representative of Formula I, wherein the R⁵ group (shown as R) and D ring varies, are contemplated herein. It is herein contemplated that the identical compounds illustrated below, but each compound having the amide bond para to the urea on the central phenyl ring, are representative of compounds of Formula II of the present invention. 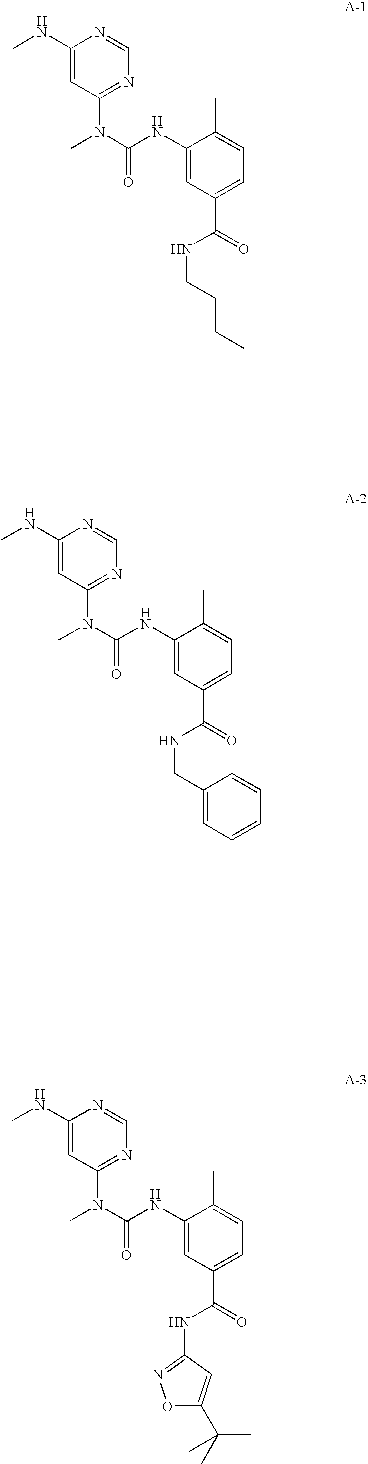 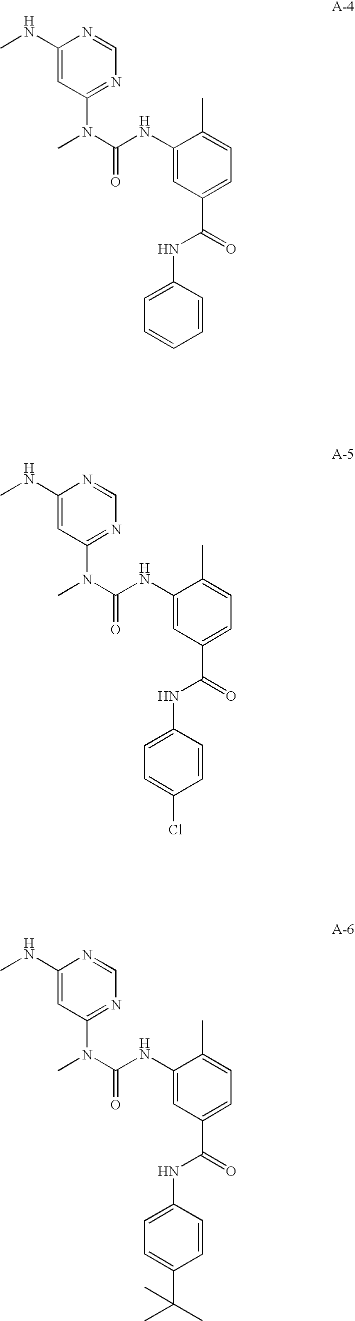 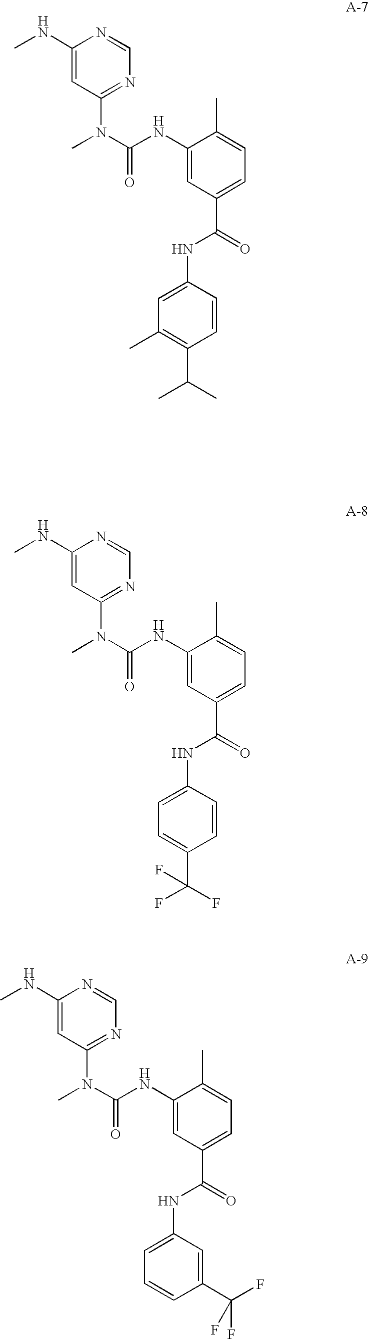 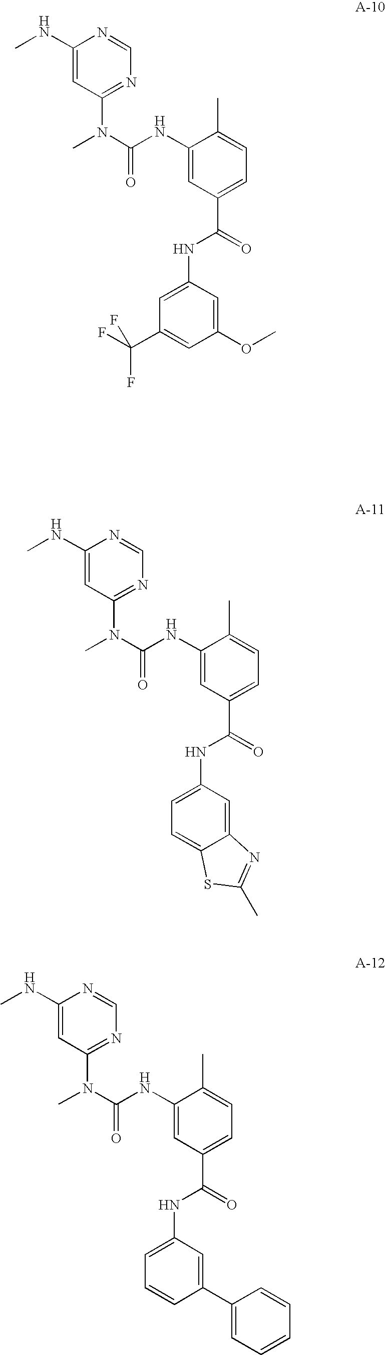 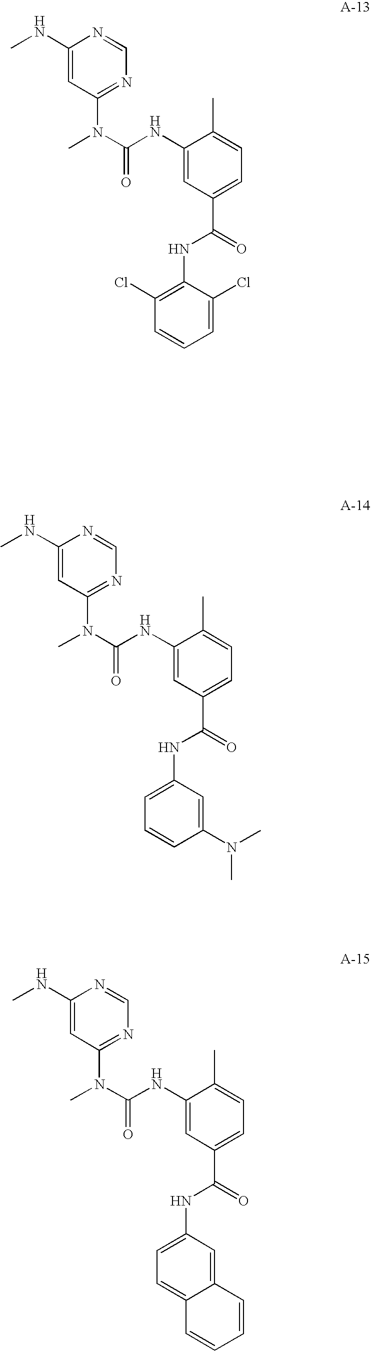 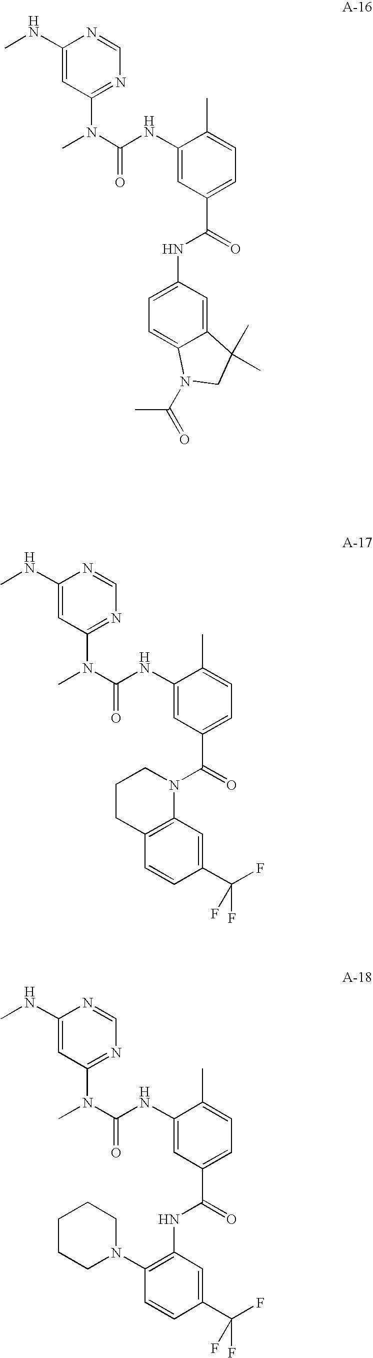 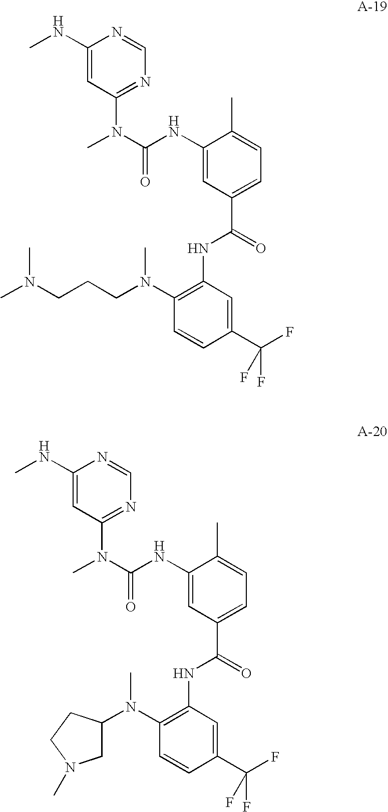 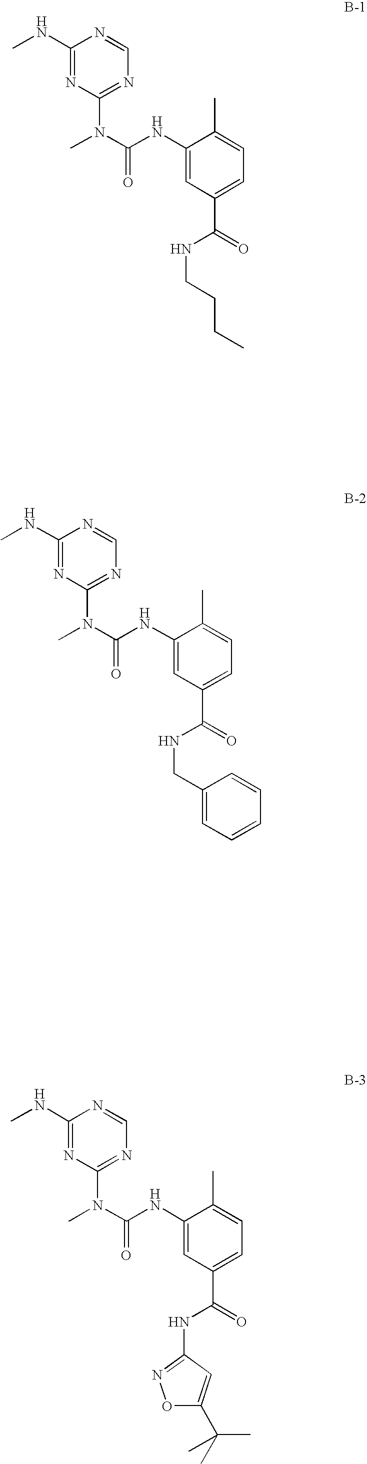 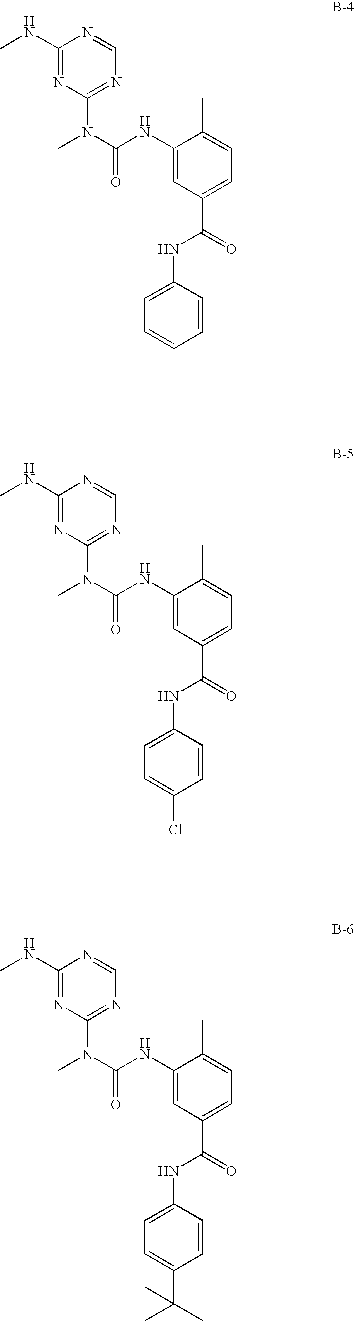 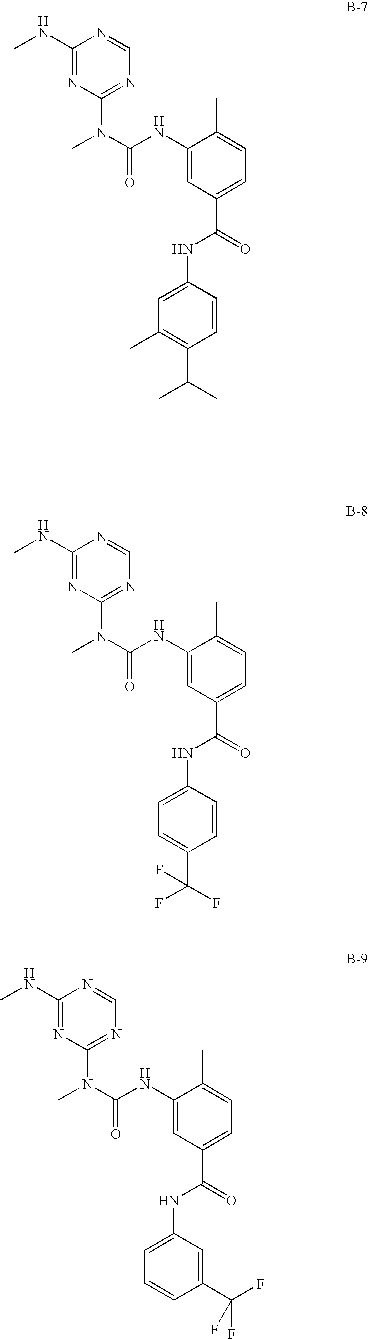 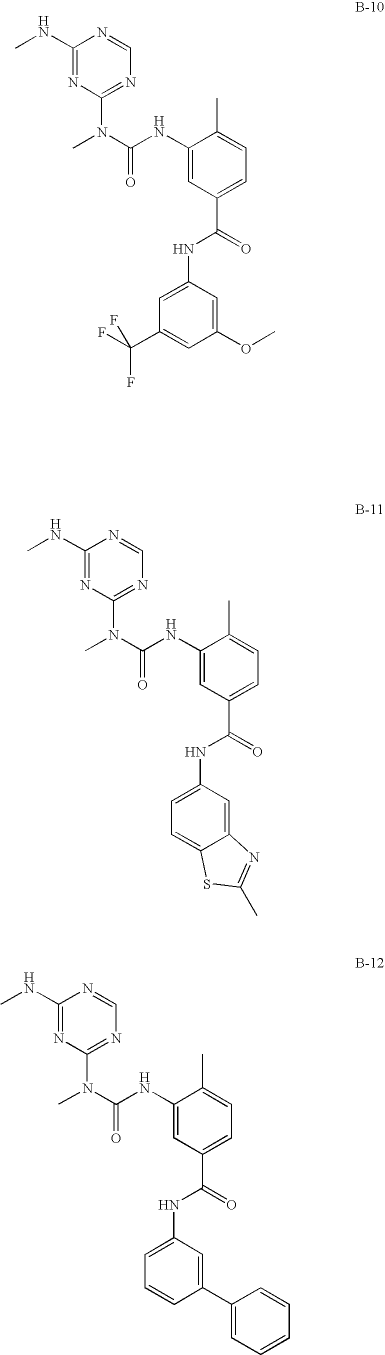 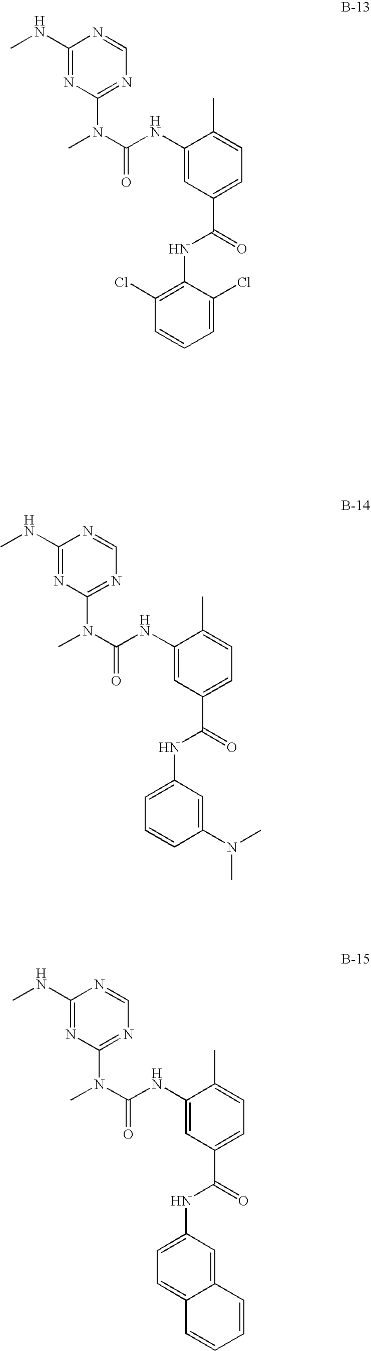 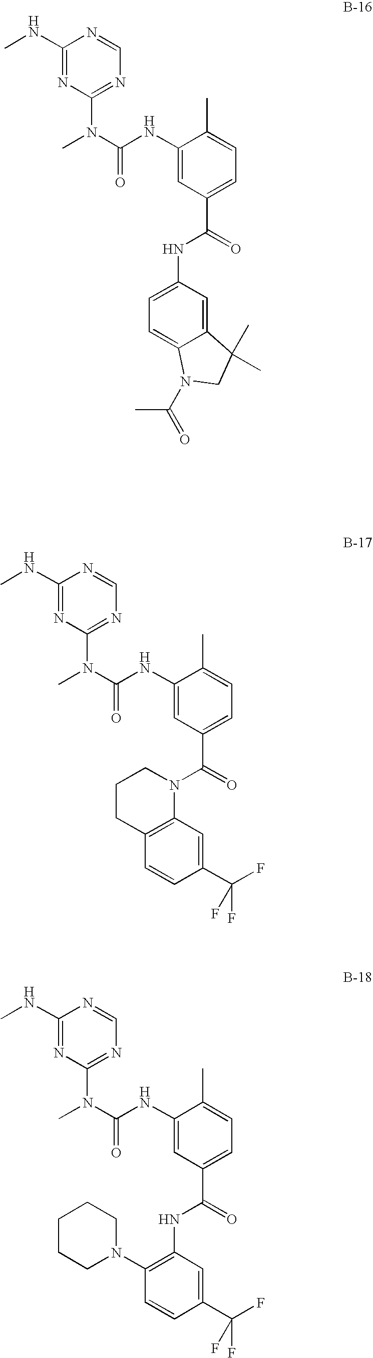 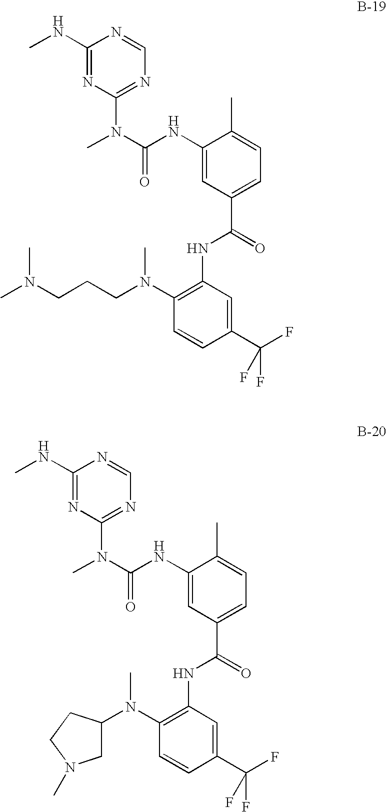 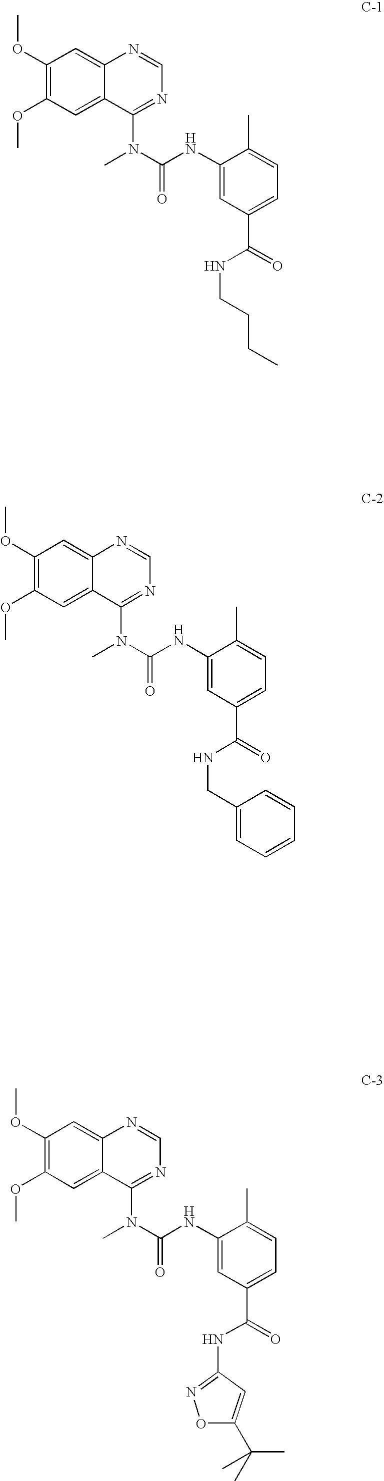 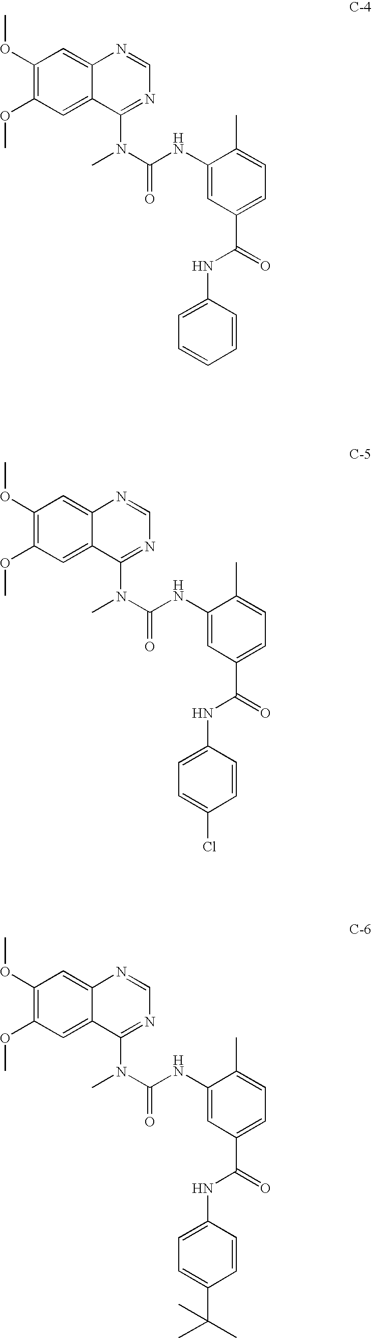 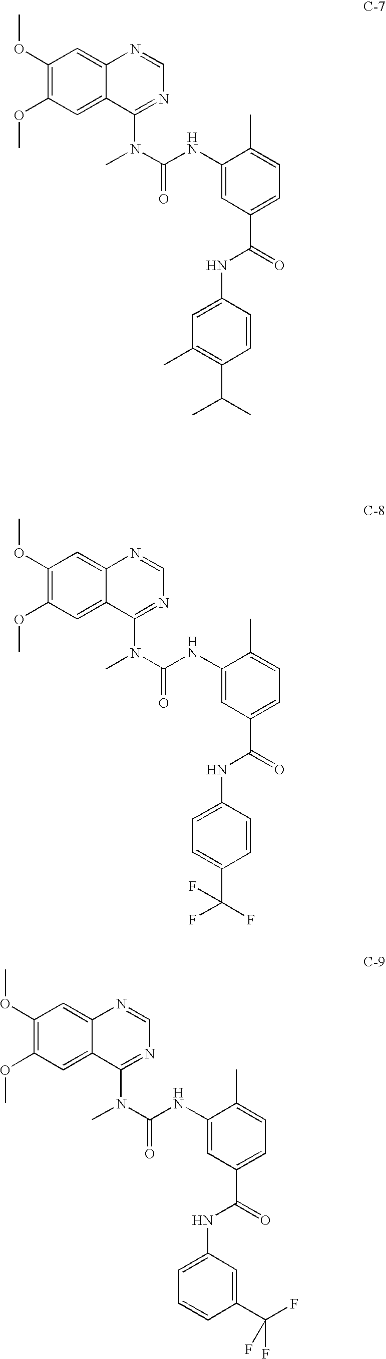 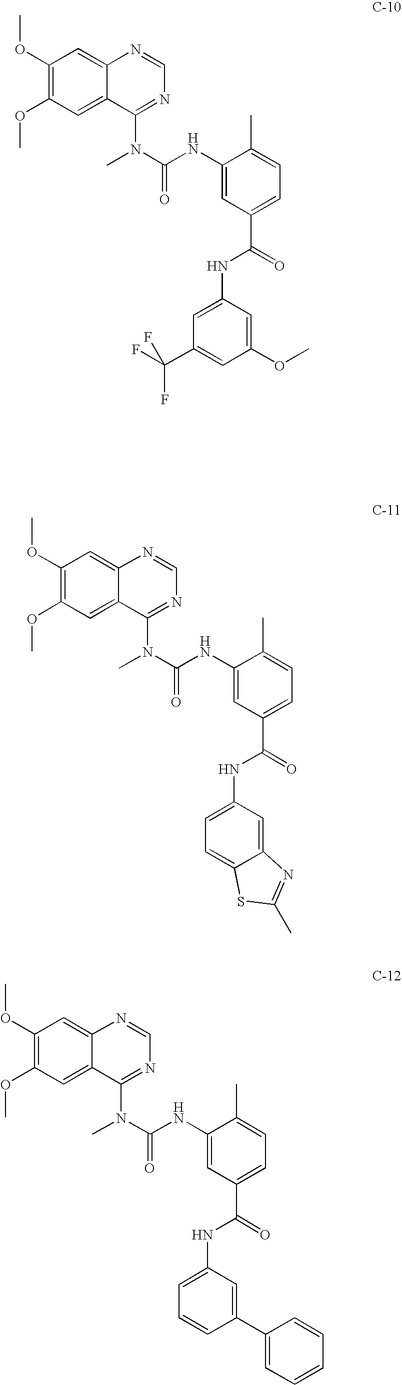 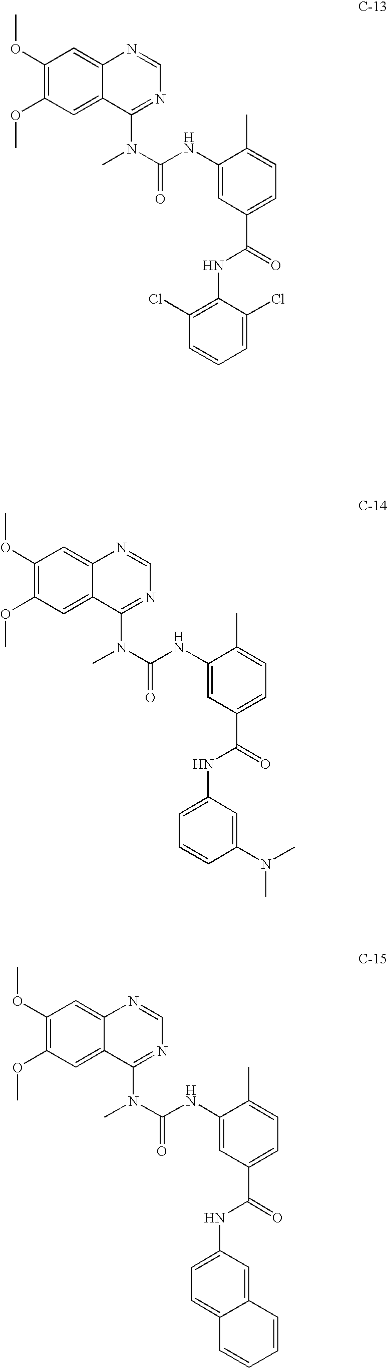 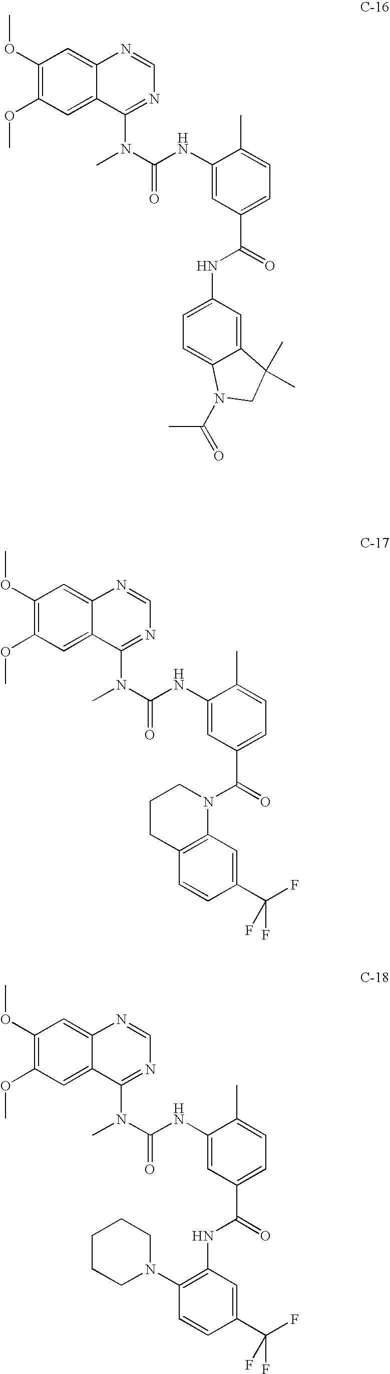 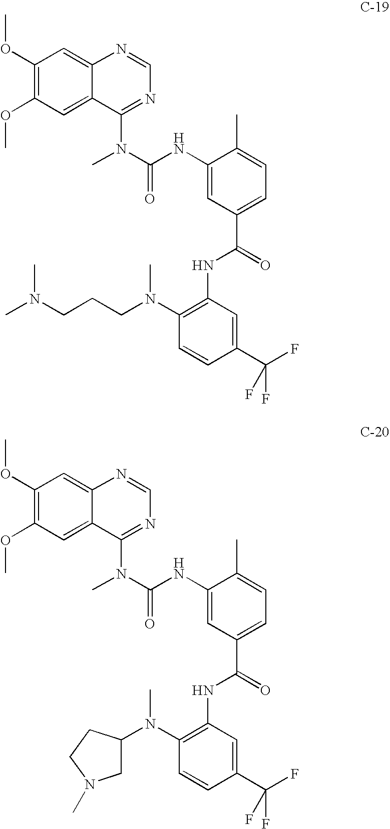 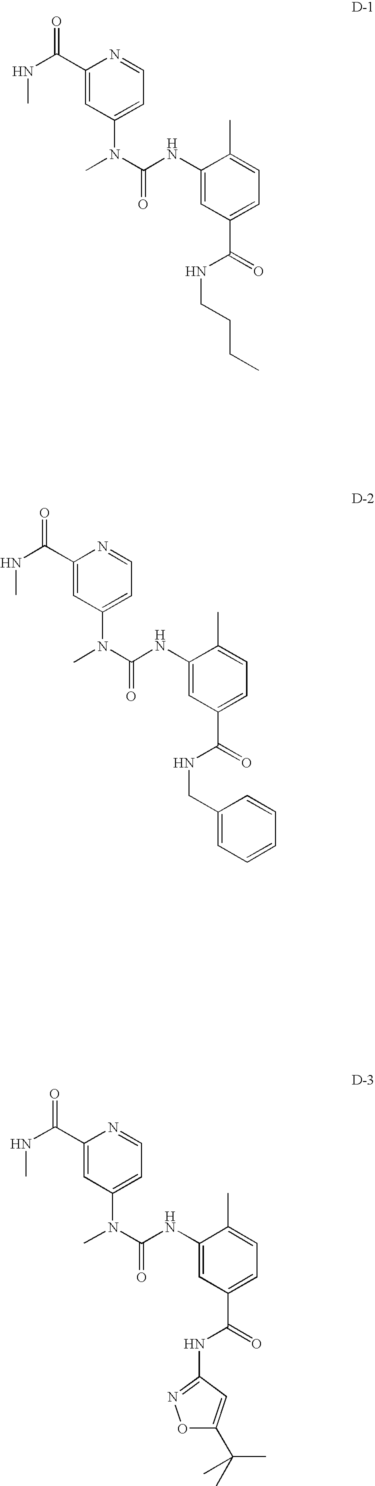 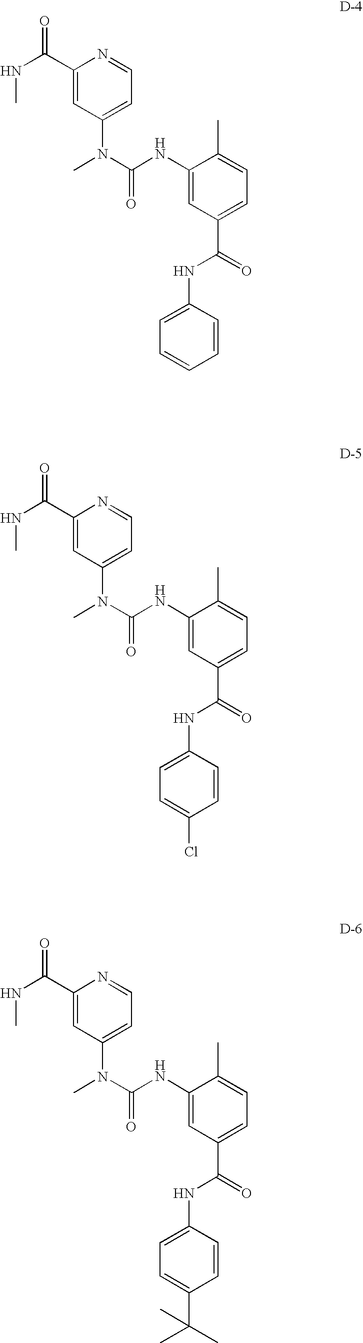 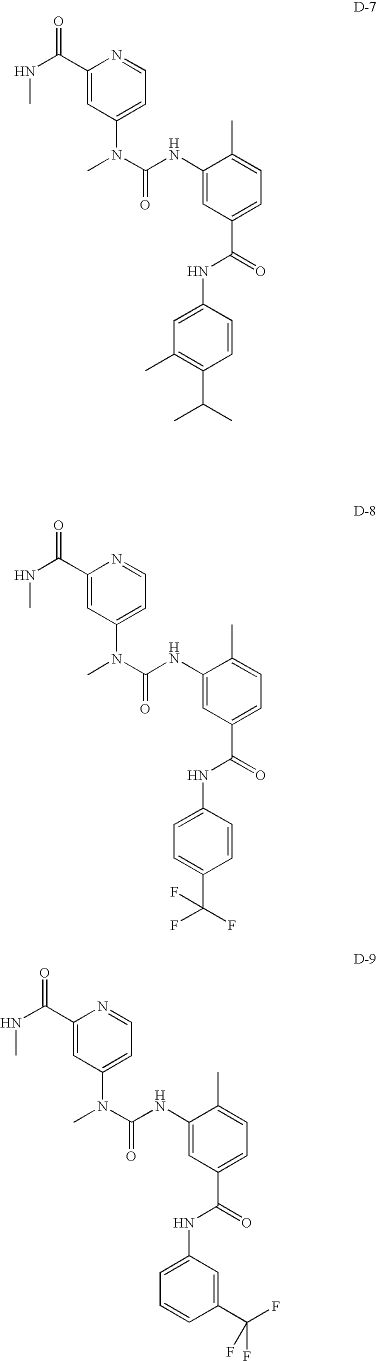 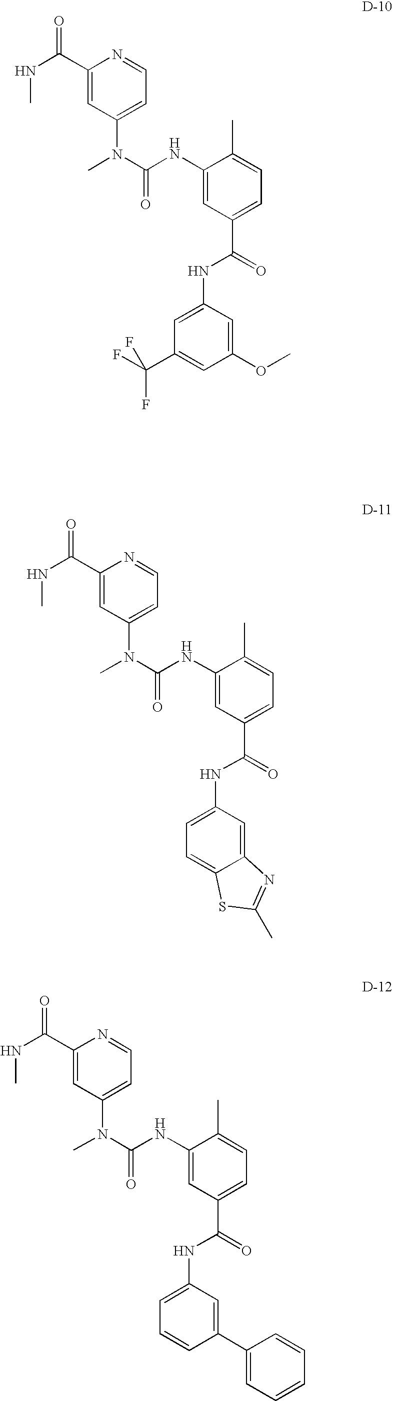 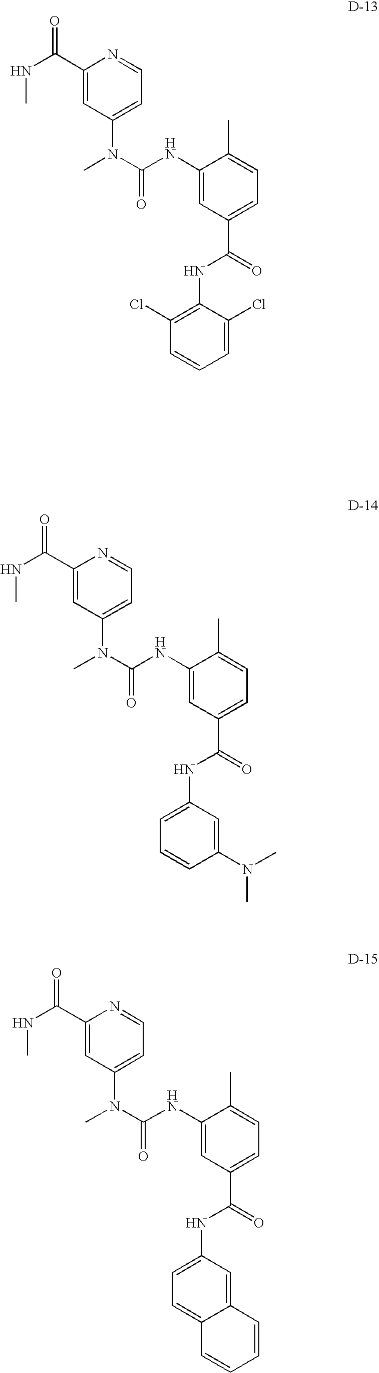 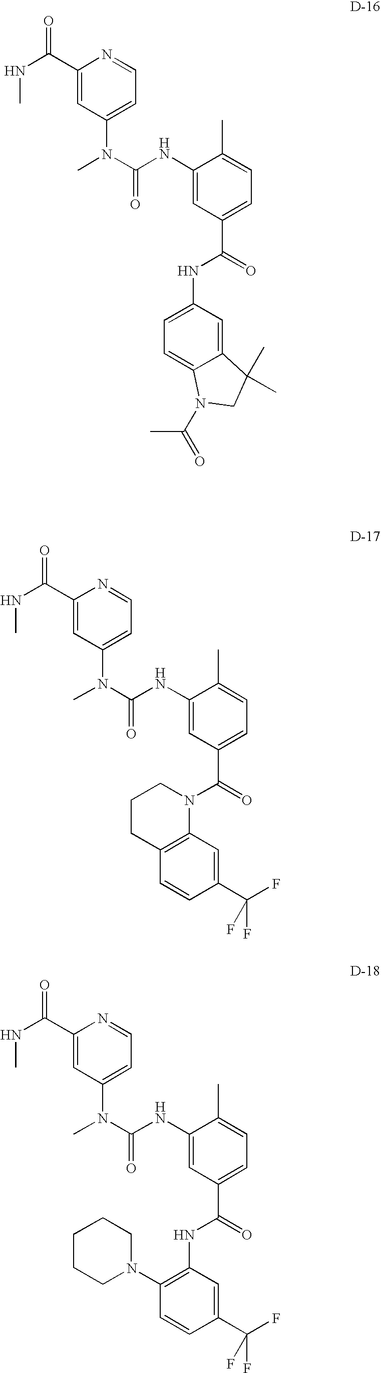 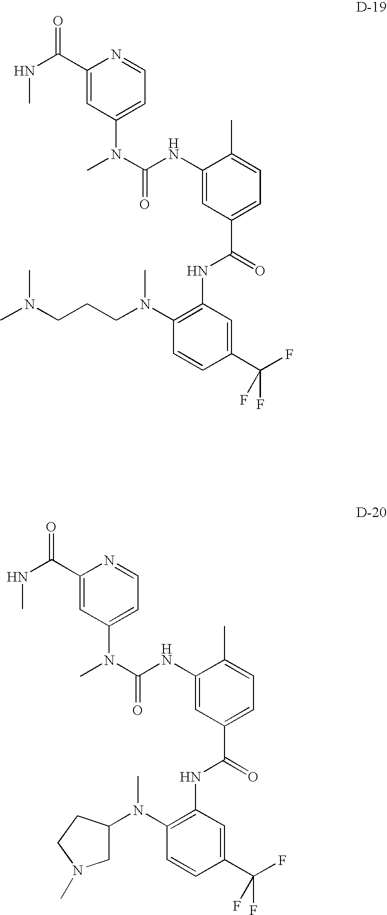 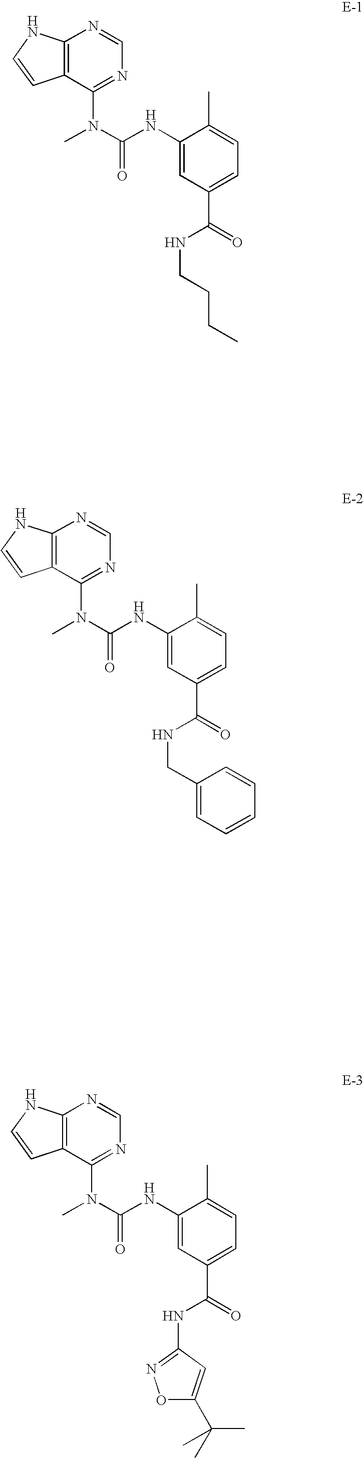 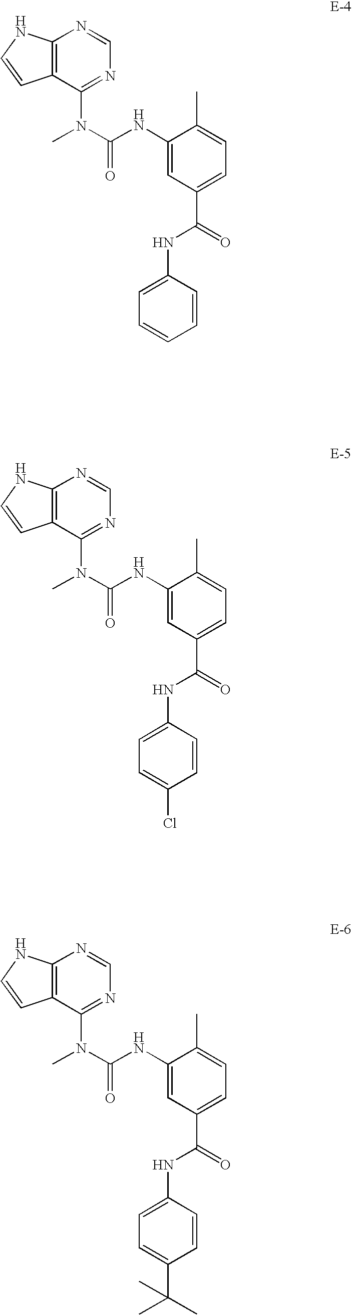 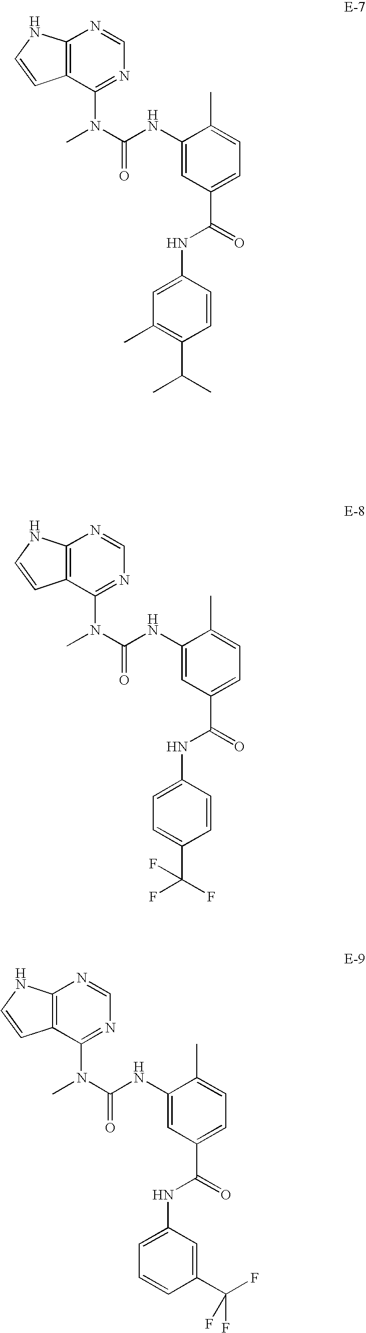 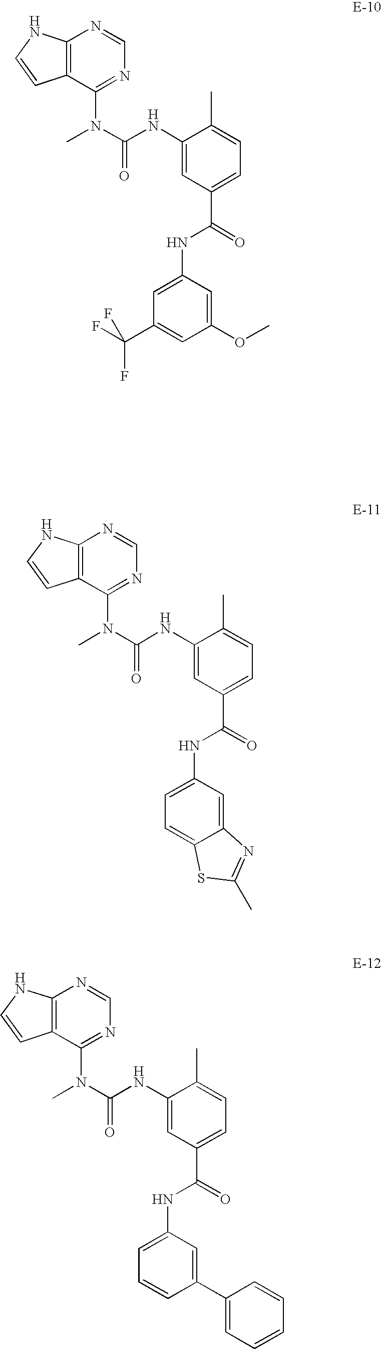 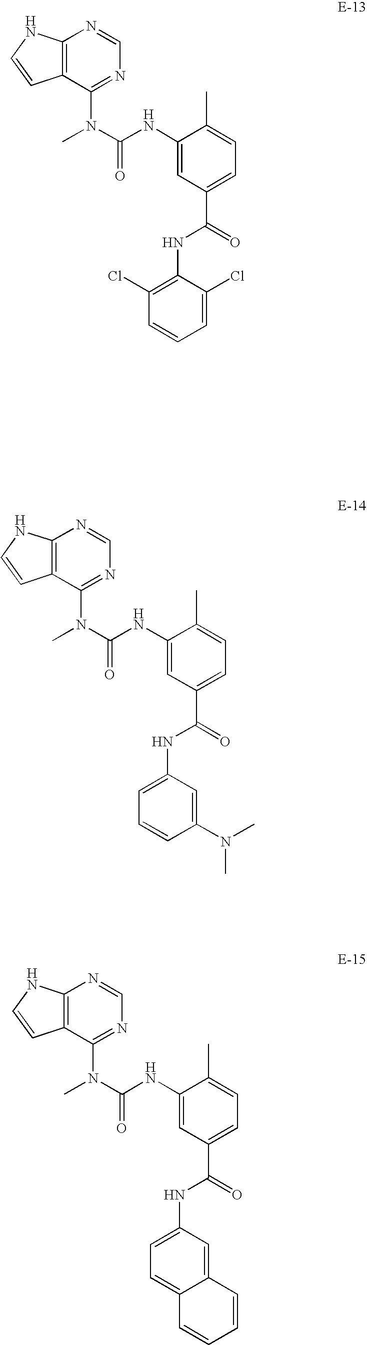 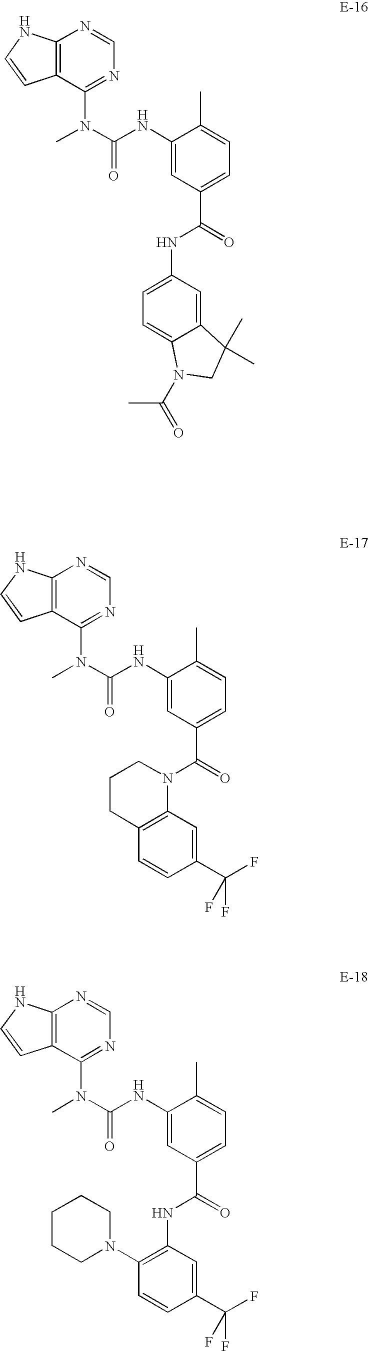 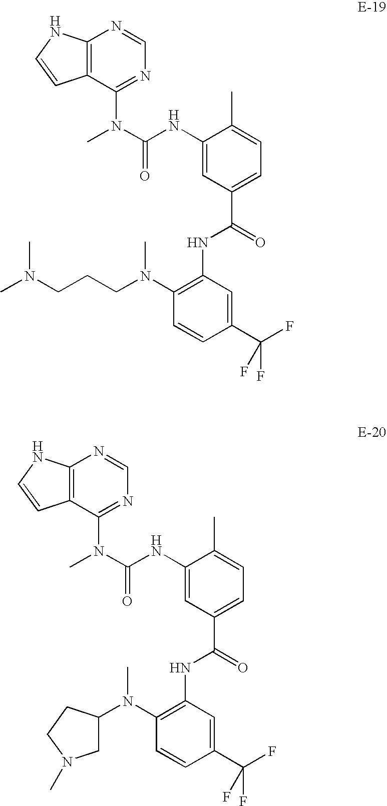
Analytical Methods:

Unless otherwise indicated, the reactions were monitored by TLC: Merck (silica gel Si-60 F₂₅₄, 0.25 mm) and purified by Flash chromatography using Merck silica gel Si-60 (230-400 mesh).

LC-MS Method:

The final product compounds were analyzed using analytical HPLC: column (Develosil RPAq 4.6×50 mm), flow: 1.5 ml/min; UV detection at 220 nm and 254 nm; with one of the following solvent gradients:
A: 5% MeCN, 95% H₂O (0.1% TFA) to 100% MeCN in 5 min
B: 10% MeCN, 90% H₂O (0.1% TFA) to 100% MeCN in 5 min
C: 20% MeCN, 80% H₂O (0.1% TFA) to 100% MeCN in 5 min
D: 30% MeCN, 70% H₂O (0.1% TFA) to 100% MeCN in 5 min
E: 40% MeCN, 60% H₂O (0.1% TFA) to 100% MeCN in 5 min
F: 50% MeCN, 50% H₂O (0.1% TFA) to 100% MeCN in 5 min
G: 10% MeCN, 90% H₂O (0.1% TFA) to 30% MeCN, 70% H₂O (0.1% TFA) in 5 min
H: 10% MeCN, 90% H₂O (0.1% TFA) to 40% MeCN, 60% H₂O (0.1% TFA) in 5 min
   Preparative HPLC Method:

Where indicated, compounds of interest were purified via preparative HPLC: VP100/21 Nucleosil 50-100 (Macherey-Nagel), eluting with hexane/EtOAc/MeOH or CH₂CI₂/MeOH/NH3-MeOH gradients.

Proton NMR Spectra:

Unless otherwise indicated, all ¹H NMR spectra were run on a Bruker, ¹H-NMR (300 MHz), ³C-NMR (75 MHz) in the indicated deuterated solvent at ambient temperature. The chemical shifts (S) are expressed in ppm, and the coupling constants J are reported in Hz.

Mass Spectra (MS)

Unless otherwise indicated, all mass spectral data for starting materials, intermediates and/or exemplary compounds were run on a Finnagan uinstrument and are reported as mass/charge (m/z), having an (M+H⁺) molecular ion. The molecular ion reported was obtained by atmospheric pressure chemical ionization (APCI) method. Compounds having an isotopic atom, such as bromine and the like, are reported according to the detected isotopic pattern, as appreciated by those skilled in the art. While the examples described above provide processes for synthesizing compounds of Formulas I and II, other methods may be utilized to prepare such compounds. Methods involving the use of protecting groups may be used. Particularly, if one or more functional groups, for example carboxy, hydroxy, amino, or mercapto groups, are or need to be protected in preparing the compounds of the invention, because they are not intended to take part in a specific reaction or chemical transformation, various known conventional protecting groups may be used. For example, protecting groups typically utilized in the synthesis of natural and synthetic compounds, including peptides, nucleic acids, derivatives thereof and sugars, having multiple reactive centers, chiral centers and other sites potentially susceptible to the reaction reagents and/or conditions, may be used.

The protecting groups may already be present in precursors and should protect the functional groups concerned against unwanted secondary reactions, such as acylations, etherifications, esterifications, oxidations, solvolysis, and similar reactions. It is a characteristic of protecting groups that they readily lend themselves, i.e. without undesired secondary reactions, to removal, typically accomplished by solvolysis, reduction, photolysis or other methods of removal such as by enzyme activity, under conditions analogous to physiological conditions. It should also be appreciated that the protecting groups should not be present in the end-products. The specialist knows, or can easily establish, which protecting groups are suitable with the reactions described herein.

The protection of functional groups by protecting groups, the protecting groups themselves, and their removal reactions (commonly referred to as “deprotection”) are described, for example, in standard reference works, such as J. F. W. McOmie, Protective Groups in Organic Chemistry, Plenum Press, London and New York (1973), in T. W. Greene, Protective Groups in Organic Synthesis, Wiley, New York (1981), in The Peptides, Volume 3, E. Gross and J. Meienhofer editors, Academic Press, London and New York (1981), in Methoden der Organischen Chemie (Methods of Organic Chemistry), Houben Weyl, 4^{th} edition, Volume 15/1, Georg Thieme Verlag, Stuttgart (1974), in H.-D. Jakubke and H. Jescheit, Aminosäuren, Peptide, Proteine (Amino Acids, Peptides, Proteins), Verlag Chemie, Weinheim, Deerfield Beach, and Basel (1982), and in Jochen Lehmann, Chemie der Kohlenhydrate: Monosaccharide und Derivate (Chemistry of Carbohydrates: Monosaccharides and Derivatives), Georg Thieme Verlag, Stuttgart (1974).

Synthetic procedures may also be carried out where functional groups of starting compounds, which are not intended to take part in the reaction, may be present in unprotected form without the added step of protecting that group by, for example, one or more of the protecting groups mentioned above or taught in the references above.

Salts of a compound of the invention having a salt-forming group may be prepared in a conventional manner or manner known to persons skilled in the art. For example, acid addition salts of compounds of the invention may be obtained by treatment with an acid or with a suitable anion exchange reagent. A salt with two acid molecules (for example a dihalogenide) may also be converted into a salt with one acid molecule per compound (for example a monohalogenide); this may be done by heating to a melt, or for example by heating as a solid under a high vacuum at elevated temperature, for example from 50° C. to 170° C., one molecule of the acid being expelled per molecule of the compound.

Acid salts can usually be converted to free-base compounds, e.g. by treating the salt with suitable basic agents, for example with alkali metal carbonates, alkali metal hydrogen carbonates, or alkali metal hydroxides, typically potassium carbonate or sodium hydroxide. Exemplary salt forms and their preparation are described herein in the Definition section of the application.

All synthetic procedures described herein can be carried out under known reaction conditions, advantageously under those described herein, either in the absence or in the presence (usually) of solvents or diluents. As appreciated by those of ordinary skill in the art, the solvents should be inert with respect to, and should be able to dissolve, the starting materials and other reagents used. Solvents should be able to partially or wholly solubilize the reactants in the absence or presence of catalysts, condensing agents or neutralizing agents, for example ion exchangers, typically cation exchangers for example in the H⁺ form. The ability of the solvent to allow and/or influence the progress or rate of the reaction is generally dependent on the type and properties of the solvent(s), the reaction conditions including temperature, pressure, atmospheric conditions such as in an inert atmosphere under argon or nitrogen, and concentration, and of the reactants themselves.

Suitable solvents for conducting reactions to synthesize compounds of the invention include, without limitation, water; esters, including lower alkyl-lower alkanoates, e.g., EtOAc; ethers including aliphatic ethers, e.g., Et₂O and ethylene glycol dimethylether or cyclic ethers, e.g., THF; liquid aromatic hydrocarbons, including benzene, toluene and xylene; alcohols, including MeOH, EtOH, 1-propanol, IPOH, n- and t-butanol; nitriles including CH₃CN; halogenated hydrocarbons, including CH₂Cl₂, CHCl₃ and CCl₄; acid amides including DMF; sulfoxides, including DMSO; bases, including heterocyclic nitrogen bases, e.g. pyridine; carboxylic acids, including lower alkanecarboxylic acids, e.g., AcOH; inorganic acids including HCl, HBr, HF, H₂SO₄ and the like; carboxylic acid anhydrides, including lower alkane acid anhydrides, e.g., acetic anhydride; cyclic, linear, or branched hydrocarbons, including cyclohexane, hexane, pentane, isopentane and the like, and mixtures of these solvents, such as purely organic solvent combinations, or water-containing solvent combinations e.g., aqueous solutions. These solvents and solvent mixtures may also be used in “working-up” the reaction as well as in processing the reaction and/or isolating the reaction product(s), such as in chromatography.

The invention further encompasses “intermediate” compounds, including structures produced from the synthetic procedures described, whether isolated or not, prior to obtaining the finally desired compound. Structures resulting from carrying out steps from a transient starting material, structures resulting from divergence from the described method(s) at any stage, and structures forming starting materials under the reaction conditions are all “intermediates” included in the invention. Further, structures produced by using starting materials in the form of a reactive derivative or salt, or produced by a compound obtainable by means of the process according to the invention and structures resulting from processing the compounds of the invention in situ are also within the scope of the invention.

New starting materials and/or intermediates, as well as processes for the preparation thereof, are likewise the subject of this invention. In select embodiments, such starting materials are used and reaction conditions so selected as to obtain the desired compound(s).

Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups may be protected with suitable protecting groups when necessary. Protecting groups, their introduction and removal are described above.

Compounds of the present invention can possess, in general, one or more asymmetric carbon atoms and are thus capable of existing in the form of optical isomers as well as in the form of racemic or non-racemic mixtures thereof. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using optically active starting materials. These isomers may be in the form of a free acid, a free base, an ester or a salt. All such isomeric forms of these compounds are expressly included in the present invention.

The compounds of this invention may also be represented in multiple tautomeric forms. The invention expressly includes all tautomeric forms of the compounds described herein.

The compounds may also occur in cis- or trans- or E- or Z-double bond isomeric forms. All such isomeric forms of such compounds are expressly included in the present invention. All crystal forms of the compounds described herein are expressly included in the present invention.

Substituents on ring moieties (e.g., phenyl, thienyl, etc.) may be attached to specific atoms, whereby they are intended to be fixed to that atom, or they may be drawn unattached to a specific atom, whereby they are intended to be attached at any available atom that is not already substituted by an atom other than H (hydrogen).

The compounds of this invention may contain heterocyclic ring systems attached to another ring system. Such heterocyclic ring systems may be attached through a carbon atom or a heteroatom in the ring system.

Alternatively, a compound of any of the formulas described herein may be synthesized according to any of the procedures described herein. In the procedures described herein, the steps may be performed in an alternate order and may be preceded, or followed, by additional protection/deprotection steps as necessary. The procedures may further use appropriate reaction conditions, including inert solvents, additional reagents, such as bases (e.g., LDA, DIEA, pyridine, K₂CO₃, and the like), catalysts, and salt forms of the above. The intermediates may be isolated or carried on in situ, with or without purification. Purification methods are known in the art and include, for example, crystallization, chromatography (liquid and gas phase, and the like), extraction, distillation, trituration, reverse phase HPLC and the like. Reactions conditions such as temperature, duration, pressure, and atmosphere (inert gas, ambient) are known in the art and may be adjusted as appropriate for the reaction.

As can be appreciated by the skilled artisan, the above synthetic schemes are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. Synthetic chemistry transformations and protecting group methodologies (protection and deprotection) useful in synthesizing the inhibitor compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3^{rd} edition, John Wiley and Sons (1999); L. Fieser and M. Fieser, Fieser and Fieser's Reagents for Organic Synthesis, John Wiley and Sons (1994); A. Katritzky and A. Pozharski, Handbook of Heterocyclic Chemistry, 2^{nd} edition (2001); M. Bodanszky, A. Bodanszky, The Practice of Peptide Synthesis, Springer-Verlag, Berlin Heidelberg (1984); J. Seyden-Penne, Reductions by the Alumino- and Borohydrides in Organic Synthesis, 2^{nd} edition, Wiley-VCH, (1997); and L. Paquette, editor, Encyclopedia of Reagents for Organic Synthesis, John Wiley and Sons (1995).

The compounds of the invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. By way of example, a compound of the invention may be modified to incorporate a hydrophobic group or “greasy” moiety in an attempt to enhance the passage of the compound through a hydrophobic membrane, such as a cell wall.

These detailed descriptions fall within the scope, and serve to exemplify, the above-described General Synthetic Procedures which form part of the invention. These detailed descriptions are presented for illustrative purposes only and are not intended as a restriction on the scope of the invention.

Although the pharmacological properties of the compounds of the invention (Formulas I and II) vary with structural change, in general, activity possessed by compounds of Formulas I and II may be demonstrated both in vitro as well as in vivo. Particularly, the pharmacological properties of the compounds of this invention may be confirmed by a number of pharmacological in vitro assays. The following exemplified pharmacological assays have been carried out with the compounds according to the invention. Compounds of the invention were found to inhibit the activity of various kinase enzymes, including, without limitation, Tie-2, Lck, p38 and VEGF receptor kinases at doses less than 25 μM.

### BIOLOGICAL EVALUATION

The following assays can be employed to determine the degree of activity of a compound as a protein kinase inhibitor. Compounds described herein have been tested in one or more of these assays, and have shown activity. Representative compounds of the invention were tested and found to exhibit IC₅₀ values of at least <25 μM in any one of the described assays, thereby demonstrating and confirming the utility of the compounds of the invention as protein kinase inhibitors and in the prophylaxis and treatment of immune diseases, proliferative disorders, angiogenic diseases, etc.

### Tie-2—Homogenous Time Resolved Flourescent (HTRF) Kinase Assay

IC₅₀'s for the inhibition of the Tie-2 kinase enzyme for individual compounds were measured using an HTRF assay, utilizing the following procedure:

In a 96 well plate (available from Costar Co.) was placed 1 uL of each test and standard compound per well in 100% DMSO having a 25 uM final compound concentration (3-fold, 10 point dilution). To each well was added 20 uL of a reaction mix formed from Tie-2 (4.0 uL; of a 10 mM stock solution available from Gibco), 0.05% BSA (0.1 uL; from a 10% stock solution available from Sigma-Aldrich Co.), 0.002 mM of BLC HER-2 KKK (Biotinylated Long chain peptide; 0.04 uL; from a 0.002 mM stock solution), 0.01 mM concentration of ATP (0.02 uL; commercially available from Sigma-Aldrich Co.) and the remaining solution was water (15.84 uL) to make to a total volume of 20 uL/well.

The reaction was initiated in each well by adding 20 uL per well of an enzyme preparation consisting of a 50 mM concentration of Hepes (1.0 uL; from a 1000 mM stock solution commercially available from Gibco Co.), 0.05% concentration of BSA (0.1 uL), 4 mM of DTT (0.08 uL; from a 1000 mM stock solution available from Sigma-Aldrich Co.), a 2.4×10⁻⁷ concentration of Tie-2 (0.02 uL, from a 4 mM concentration stock), with the remaining volume being water (18.8 uL) to dilute the enzyme preparation to a total volume of 20 uL. The plate was incubated for about 90 minutes at RT. After incubation, a 160 uL of a filtered detection mixture, prepared from 0.001 mg/ml of SA-APC (0.0765 uL; available as a 2.09 mg/ml stock solution from Gibco), 0.03125 nM concentration of Eu-Ab (0.1597 uL; available in a 31.3 nM stock solution from Gibco), with the remaining volume being Detection buffer (159.73 uL), was added to each well to stop the reaction therein. The plate was then allowed to equilibrate for about 3 hr and read on a Ruby Star fluorescent reader (available from BMG Technologies, Inc.) using a 4 parameter fit using activity base to calculate the corresponding IC₅₀'s for the test and standard compounds in each well. Of the compounds tested, the following exemplary compounds were found to have IC₅₀'s for the inhibition of Tie-2 as measured by the HTRF assay of less than or equal to 5 uM: Examples 16-33, 35-38, 40, 42, 43, 45, 49, 51, 54, 71 and 73.

### Tie-2 Cell-Based Delfia Assay

Day 1—Plate Preparation

Three 175 ml flasks of EAHY926 cells were obtained from the University of N. Carolina. All cells were trypsinized (i.e., washed with 20 mL of PBS followed by 3 mL of trypsin-EDTA obtained from Gibco Co., cat. no. 25300-054, for 5 min at RT), then cultured in a growth medium solution containing DMEM (High glucose, Gibco Co., cat. no. 1965-092), 10% FBS serum (Gibco Co., cat. no. 10099-141) and P/S (Penicillin-Streptomycin-Glutamine; Gibco Co., cat. no. 10378-016) culture media. The cells were counted using a Z2® coulter® counter. The cells were plated in four 24-well tissue culture plates (Costar Co., cat. no. 353047) to initially contain 4×10⁵ cells/ml per well, and then loaded to 500 uL volume having a final cell density of 2×10⁵ cells/well. The cells were incubated for 5 or more hours at 37° C. under 5% CO₂. The DMEM+10% serum+P/S culture media was removed and the cells washed twice with 500 uL of PBS (without Ca+ and Mg++; Gibco Co., cat. no. 14190-136) at RT. 500 uL of 0.5% FBS+F12 (F12 nutrient mixture; Gibco Co., cat. no. 11765-054) was added to each well and the cells were incubated at 37° C. overnight (about 15 hr).

100 ug of anti-hTie2 antibody (R & D Systems, Inc., Cat. No. AF313) was diluted with 10 mL of ice-cold PBS to prepare a 10 ug/mL antibody concentration stock. A 96-well microplate (Perkin-Elmer Wallac, cat. no. AAAND-0001) was coated with 100 uL of the anti-Tie2 antibody stock and the coated plate was stored at 4° C. overnight.

Day 2—Compound Plate Preparation

The media in the microplate was replaced with a preparation of 500 uL DMEM+1% BSA (Bovine Serum Albumin; ICN Biomedicals, Inc., cat. no. 160069). 20 uL of a selected Tie2 reference compound was placed in a selected well of the 96-well plate, and diluted 1:4 with 100% DMSO from an initial concentration of about 10 mM to a final concentration of about 2.5 mM, then diluted 1:3 with 100% DMSO for a 10 point dilution to a final concentration of about 0.128 uM.

Test compounds (10 uL of a 10 mM concentration) were similarly diluted 1:4 with 100% DMSO to obtain a sample concentration of about 2.5 mM, then diluted 1:3 for a 10 point dilution to finally obtain a concentration of about 0.128 uM for each test compound. 20 uL of 100% DMSO served as positive controls, while and 10 uL of the 2.5 mM concentration of the reference compound served as the negative control.

A 2 uL aliquot from each well (test compounds, positive and negative controls) in the 96-well plate was added to designated wells in the 24-well cell culture plate (1:250). The culture plate was incubated for 2.5 at 37° C. in an atmosphere of about 5% CO₂.

The Tie-2 ligand was stimulated with the following series of preparations: (1) about 0.5 mL of a protease inhibitor cocktail (Sigma-Aldrich Co., cat. no. P8340) was thawed; (2) to prepare the phosphatase inhibitor, a 300 mM NaVO₄ (Sigma-Aldrich Chem. Co., cat. no. S6508-10G) stock solution in PBS was made and stored at RT. Two 1 mL aliquots of the NaVO₄ solution was prepared in separate two vials by adding 100 uL of the NaVO₄ stock solution to 900 uL RT PBS and each solution was activated by adding 6 uL of H₂O₂ to each vial. Both NaVO₄ solutions were mixed, wrapped in aluminum foil and stored at RT for 15 min.

The Delfia plates, containing 200 uL of PBS+0.1% TWEEN20, were washed three times and blocked by adding 200 uL of a diluted solution of 5% BSA (16 mL of stock 7.5% BSA solution, available from Perkin-Elmer Wallac, Cat. No. CR84-100, was diluted with 8 mL of room temperature PBS). The plates were then stored at room temperature for about one hour.

100 uL of 35% BSA solution was diluted with 3.4 mL of ice cold PBS to make a 1% BSA/PBS solution. 100 uL of this 1% BSA/PBS solution was diluted with 900 uL of ice cold PBS. hAng1 was reconstituted with 250 uL of ice cold PBS+0.1% BSA to make a 100 ug/mL concentration in solution. The solution was separated into 70 uL aliquots and stored at −80° C.

1 mL of the 30 mM solution of NaVO₄/PBS was diluted with 99 mL of ice cold PBS to form a 300 uM concentration. The solution was kept cold on ice. 210 uL of the activated NaVO₄ and 280 uL of the protease inhibitor preparation was added to 21 mL of RIPA buffer and kept cold on ice.

Dilute hAng1 and Stimulate Cells:

70 uL of the 100 ug/mL stock solution was added to 700 uL in 1% BSA/DMEM (1:10) to 10 ug/mL concentration, and it was stored on ice. 5 uL of this 10 ug/mL hAng1 preparation was added to each well of the 24-well plate. The plate was shaken at 700 rpm at 37° C. for about 2.5 minutes.

After shaking, the wells were incubated for 7.5 min at 37° C. The media was removed and 400 uL of ice cold PBS+300 uM NaVO₄ was added. The wells were kept on ice for at least 5 min and washed 1× with ice cold PBS+300 uM NaVO₄. The wells were tapped against a dry paper towel. The cells were lysed with 150 uL of RIPA, 300 uM of NaVO₄, and 100 uL/1*10⁷ cells protease inhibitor cocktail (purchased from Sigma-Aldrich, Cat. No. P8340). The solution was incubated, then shaken on ice for 30 min.

The BSA blocking solution was removed from the 96-well plates, which were then tapped dry. 140 uL of cell lysate was added to the antibody-coated plate and the plate was incubated at 4° C. for 2 hours.

Delfia 25× Wash Buffer Concentrate (purchased from Perkin-Elmer Wallac, Cat. No. 1244-114) was diluted with 24 parts DDI water to obtain a washing solution. The lysate was removed and the plate was washed three times each with 400 uL of Delfia washing solution. The plate was tap dried with a paper towel.

The Anti-Phosphotyrosine clone 4G10 (purchased from Upstatebiotech Co., Cat. No. 05-321) was diluted with Delfia Assay Buffer (purchased from Perkin-Elmer Wallac, cat. no. 1244-1111) to make a solution of about 1 ug/mL in concentration. 100 uL of antibody was added to the plate and the plate was incubated at room temperature for one hour. The plate was again washed three times with 400 uL pre-time of the Delfia Washing solution.

The Eu-N1 labeled anti-mouse antibody (purchased from Perkin-Elmer Wallac, cat. no. AD0124) was diluted with Delfia Assay Buffer to make a solution of about 0.1 ug/mL in concentration.

100 uL of antibody was added to the plate and the plate was incubated at room temperature for one hour. The plate was again washed with Delfia Wash Buffer three times as described above. 100 uL of Delfia Enhancement Solution (purchased from Perkin-Elmer Wallac, Cat. No. 1244-105) was added to each well and the plate was incubated at room temperature for 5 min in the dark.

The Europium signal was measured with a Victor multilabel counter (Wallac Model 1420) while shaking (shake fast, linear, 0.10 mm for is) using a Europium protocol.

Raw data was analyzed using a fit equation in XLFit. IC₅₀ values were then determined using Grafit software. Each of the examples described herein exhibited activity in the HTRF assay and the delfia cell-based assay with IC₅₀ values less than 10.0 μM.

The compounds of the invention also were found to have inhibitory activity with respect to other kinase enzymes as well. For example, the compounds were found to be inhibitors of Lck, p38 and/or VEGF. The exemplary assays described as follows were used to make such determination.

### LCK-Homogenous Time Resolved Flourescent (HTRF) Kinase Assay

The LCK HTRF assay begins with LCK in the presence of ATP phosphorylating the biotinylated peptide Gastrin. The reaction incubates for 90 min. To quench the assay detection reagents are added which both stop the reaction by diluting out the enzyme and chelating the metals due to the presence of EDTA. Once the detection reagents are added the assay incubates for 30 min to allow for equilibration of the detection reagents.

The LCK HTRF assay is comprised of 10 μL of compound in 100% DMSO, 15 μL of ATP and biotinylated Gastrin, and 15 μL of LCK KD GST (225-509) for a final volume of 40 μL. The final concentration of gastrin is 1.2 μM. The final concentration of ATP is 0.5 μM (Km app=0.6 μM+/−0.1) and the final concentration of LCK is 250 pM. Buffer conditions are as follows: 50 mM HEPES pH 7.5, 50 mM NaCl, 20 mM MgCl, 5 mM MnCl, 2 mM DTT, 0.05% BSA.

The assay is quenched and stopped with 160 μL of detection reagent. Detection reagents are as follows: Buffer made of 50 mM Tris, pH 7.5, 100 mM NaCl, 3 mM EDTA, 0.05% BSA, 0.1% Tween20. Added to this buffer-prior to reading is Steptavidin allophycocyanin (SA-APC) at a final conc in the assay of 0.0004 mg/mL, and europilated anti-phosphotyrosine Ab (Eu-anti-PY) at a final conc of 0.025 nM.

The assay plate is read in either a Discovery or a RubyStar. The eu-anti-PY is excited at 320 nm and emits at 615 nm to excite the SA-APC which in turn emits at 655 nm. The ratio of SA-APC at 655 nm (excited due to close proximity to the Eu-anti-PY because of phosphorylation of the peptide) to free Eu-anti-PY at 615 nm will give substrate phosphorylation.

Assays for other kinases are done in a similar way as described above, varying the concentrations of enzyme, peptide substrate, and ATP added to the reaction, depending on the specific activity of the kinase and measured Km's for the substrates.

Human Mixed Lymphocyte Reaction (huMLR):

The purpose of this assay is to test the potency of T cell activation inhibitors in an in vitro model of allogeneic T cell stimulation. Human peripheral blood lymphocytes (hPBL; 2×10⁵/well) are incubated with mitomycin C-treated B lymphoblastoid cells (JY cell line; 1×10⁵/well) as allogeneic stimulators in the presence or absence of dilutions of potential inhibitor compound in 96-well round-bottom tissue culture plates. These cultures are incubated at 37° C. in 5% CO₂ for 6 days total. The proliferative response of the hPBL is measured by ³H-thymidine incorporation overnight between days 5 and 6 after initiation of culture. Cells are harvested onto glass fiber filters and ³H-thymidine incorporation into DNA is analyzed by liquid scintillation counter.

Jurkat Proliferation/Survival Assay:

The purpose of this assay is to test the general anti-proliferative/cytotoxic effect of compounds on the Jurkat human T cell line. Jurkat cells (1×10⁵/well) are plated in 96-well flat-bottom tissue culture plates with or without compound dilutions and cultured for 72 h at 37° C. in 5% CO₂. Viable cell number is determined during the last 4 h of culture by adding 10 μL/well WST-1 dye. WST-1 dye conversion relies on active mitochondrial electron transport for reduction of the tetrazolium dye. The dye conversion is read by OD at 450-600 nm.

Anti-CD3/CD28-Induced T Cell IL-2 Secretion and Proliferation Assay:

The purpose of this assay is to test the potency of T cell receptor (TCR; CD3) and CD28 signaling pathway inhibitors in human T cells. T cells are purified from human peripheral blood lymphocytes (hPBL) and pre-incubated with or without compound prior to stimulation with a combination of an anti-CD3 and an anti-CD28 antibody in 96-well tissue culture plates (1×10⁵ T cells/well). Cells are cultured for ˜20 h at 37° C. in 5% CO₂, then secreted IL-2 in the supernatants is quantified by cytokine ELISA (Pierce/Endogen). The cells remaining in the wells are then pulsed with ³H-thymidine overnight to assess the T cell proliferative response. Cells are harvested onto glass fiber filters and ³H-thymidine incorporation into DNA is analyzed by liquid scintillation counter. For comparison purposes, phorbol myristic acid (PMA) and calcium ionophore can be used in combination to induce IL-2 secretion from purified T cells. Potential inhibitor compounds can be tested for inhibition of this response as described above for anti-CD3 and -CD28 antibodies.

Assays for other kinases are done in a similar way as described above, varying the concentrations of enzyme, peptide substrate, and ATP added to the reaction, depending on the specific activity of the kinase and measured Km's for the substrates.

Of the compounds tested, exemplary compounds 16-40, 41-46, 48, 49, 54, 71 and 73 exhibited an average IC₅₀ value of 5 uM or less in the human HTRF assay for the inhibition of the Lck kinase enzyme.

The compounds were also found to be active inhibitors of the VEGF kinase receptor, as measured by the following described assays.

### HUVEC Proliferation Assay

Human Umbilical Vein Endothelial cells are purchased from Clonetics, Inc., as cryopreserved cells harvested from a pool of donors. These cells, at passage 1, are thawed and expanded in EBM-2 complete medium, until passage 2 or 3. The cells are trypsinized, washed in DMEM+10% FBS+antibiotics, and spun at 1000 rpm for 10 min. Prior to centrifugation of the cells, a small amount is collected for a cell count. After centrifugation, the medium is discarded, and the cells are resuspended in the appropriate volume of DMEM+10% FBS+antibiotics to achieve a concentration of 3×10⁵ cells/mL. Another cell count is performed to confirm the cell concentration. The cells are diluted to 3×10⁴ cells/mL in DMEM+10% FBS+antibiotics, and 100 μL of cells are added to a 96-well plate. The cells are incubated at 37° C. for 22 h.

Prior to the completion of the incubation period, compound dilutions are prepared. Five-point, five-fold serial dilutions are prepared in DMSO, at concentrations 400-fold greater than the final concentrations desired. 2.5 μL of each compound dilution are diluted further in a total of 1 mL DMEM+10% FBS+antibiotics (400× dilution). Medium containing 0.25% DMSO is also prepared for the 0 μM compound sample. At the 22 h timepoint, the medium is removed from the cells, and 100 μL of each compound dilution is added. The cells are incubated at 37° C. for 2-3 h.

During the compound pre-incubation period, the growth factors are diluted to the appropriate concentrations. Solutions of DMEM+10% FBS+antibiotics, containing either VEGF or bFGF at the following concentrations: 50, 10, 2, 0.4, 0.08, and 0 ng/mL are prepared. For the compound-treated cells, solutions of VEGF at 550 ng/mL or bFGF at 220 ng/mL for 50 ng/mL or 20 ng/mL final concentrations, respectively, are prepared since 10 μL of each will be added to the cells (110 μL final volume). At the appropriate time after adding the compounds, the growth factors are added. VEGF is added to one set of plates, while bFGF is added to another set of plates. For the growth factor control curves, the media on wells B4-G6 of plates 1 and 2 are replaced with media containing VEGF or bFGF at the varying concentrations (50-0 ng/mL). The cells are incubated at 37° C. for an additional 72 h.

At the completion of the 72 h incubation period, the medium is removed, and the cells are washed twice with PBS. After the second wash with PBS, the plates are tapped gently to remove excess PBS, and the cells are placed at −70° C. for at least 30 min. The cells are thawed and analyzed using the CyQuant fluorescent dye (Molecular Probes C-7026), following the manufacturer's recommendations. The plates are read on a Victor/Wallac 1420 workstation at 485 nm/530 nm (excitation/emission). Raw data is collected and analyzed using a 4-parameter fit equation in XLFit. IC₅₀ values are then determined.

Of the compounds tested, Examples 16-50 and 71 were found to have an IC₅₀ of less than 5 μM in the VEGF Huvec assay.

The following assays were used to characterize the ability of compounds of Formula I and II to inhibit the production of TNF-α and IL-1-β. The second assay measured the inhibition of TNF-α and/or IL-1-β in mice after oral administration of the test compounds.

Lipopolysaccharide-Activated Monocyte TNF Production Assay

Isolation of Monocytes

Test compounds were evaluated in vitro for the ability to inhibit the production of TNF by monocytes activated with bacterial lipopolysaccharide (LPS). Fresh residual source leukocytes (a byproduct of plateletpheresis) were obtained from a local blood bank, and peripheral blood mononuclear cells (PBMCs) were isolated by density gradient centrifugation on Ficol-Paque Plus (Pharmacia). PBMCs were suspended at 2×10⁶/ml in DMEM supplemented to contain 2% FCS, 10 mM, 0.3 mg/ml glutamate, 100 U/ml penicillin G and 100 mg/ml streptomycin sulfate (complete media). Cells were plated into Falcon flat bottom, 96 well culture plates (200 μl/well) and cultured overnight at 37° C. and 6% CO₂. Non-adherent cells were removed by washing with 200 μl/well of fresh medium. Wells containing adherent cells (˜70% monocytes) were replenished with 100 μl of fresh medium.

Preparation of Test Compound Stock Solutions

Test compounds were dissolved in DMZ. Compound stock solutions were prepared to an initial concentration of 10-50 μM. Stocks were diluted initially to 20-200 μM in complete media. Nine two-fold serial dilutions of each compound were then prepared in complete medium.

Treatment of Cells with Test Compounds and Activation of TNF Production with Lipopolysaccharide

One hundred microliters of each test compound dilution were added to microtiter wells containing adherent monocytes and 100 μl complete medium. Monocytes were cultured with test compounds for 60 min at which time 25 μl of complete medium containing 30 ng/ml lipopolysaccharide from E. coli K532 were added to each well. Cells were cultured an additional 4 hrs. Culture supernatants were then removed and TNF presence in the supernatants was quantified using an ELISA.

TNF Elisa

Flat bottom, 96 well Corning High Binding ELISA plates were coated overnight (4° C.) with 150 μL/well of 3 μg/ml murine anti-human TNF-α MAb (R&D Systems #MAB210). Wells were then blocked for 1 hr at room temperature with 200 μL/well of CaCl₂-free ELISA buffer supplemented to contain 20 mg/ml BSA (standard ELISA buffer: 20 mM, 150 mM NaCl, 2 mM CaCl₂, 0.15 mM thimerosal, pH 7.4). Plates were washed and replenished with 100 μl of test supernatants (diluted 1:3) or standards. Standards consisted of eleven 1.5-fold serial dilutions from a stock of 1 ng/ml recombinant human TNF (R&D Systems). Plates were incubated at room temperature for 1 hr on orbital shaker (300 rpm), washed and replenished with 100 μl/well of 0.5 μg/ml goat anti-human TNF-α (R&D systems #AB-210-NA) biotinylated at a 4:1 ratio. Plates were incubated for 40 min, washed and replenished with 100 μl/well of alkaline phosphatase-conjugated streptavidin (Jackson ImmunoResearch #016-050-084) at 0.02 μg/ml. Plates were incubated 30 min, washed and replenished with 200 μl/well of 1 mg/ml of p-nitrophenyl phosphate. After 30 min, plates were read at 405 nm on a Vₘₐₓ plate reader.

Data Analysis

Standard curve data were fit to a second order polynomial and unknown TNF-α concentrations determined from their OD by solving this equation for concentration. TNF concentrations were then plotted vs. test compound concentration using a second order polynomial. This equation was then used to calculate the concentration of test compounds causing a 50% reduction in TNF production.

Inhibition of LPS-Induced TNF-α Production in Mice

Male DBA/1LACJ mice were dosed with vehicle or test compounds in a vehicle (the vehicle consisting of 0.5% tragacanth in 0.03 N HCl) 30 minutes prior to lipopolysaccharide (2 mg/kg, I.V.) injection. Ninety minutes after LPS injection, blood was collected and the serum was analyzed by ELISA for TNF levels.

Of the compounds tested, the following compounds exhibit activities in the monocyte assay (LPS induced TNF release) with IC₅₀ values of 5 μM or less: Examples 16-18, 20, 23-25 and 30-32, as a determination of p38 activity.

### INDICATIONS

Accordingly, compounds of the invention are useful for, but not limited to, the prevention or treatment of inflammation, cancer and related diseases. The compounds of the invention have kinase modulatory activity in general, and kinase inhibitory activity in particular. In one embodiment of the invention, there is provided a method of modulating a protein kinase enzyme in a subject, the method comprising administering to the subject an effective dosage amount of a compound of a compound of Formulae I and II. In another embodiment, the kinase enzyme is ab1, Akt, bcr-ab1, Blk, Brk, Btk, c-kit, c-Met, c-src, c-fms, CDK1, CDK2, CDK3, CDK4, CDK5, CDK6, CDK7, CDK8, CDK9, CDK10, cRaf1, CSF1R, CSK, EGFR, ErbB2, ErbB3, ErbB4, Erk, Fak, fes, FGFR1, FGFR2, FGFR3, FGFR4, FGFR5, Fgr, flt-1, Fps, Frk, Fyn, Hck, IGF-1R, INS-R, Jak, KDR, Lck, Lyn, MEK, p38, PDGFR, PIK, PKC, PYK2, ros, tie, tie2, TRK, Yes or Zap70.

Various of the compounds of the invention have selective inhibitory activity for specific kinase receptor enzymes, including Tie-2, Lck, p38 and VEGFR/KDR. Accordingly, the compounds of the invention would be useful in therapy as antineoplasia agents, anti-inflammatory agents or to minimize deleterious effects of Tie-2, Lck, VEGF and/or p38.

Compounds of the invention would be useful for the treatment of neoplasia including cancer and metastasis, including, but not limited to: carcinoma such as cancer of the bladder, breast, colon, kidney, liver, lung (including small cell lung cancer), esophagus, gall-bladder, ovary, pancreas, stomach, cervix, thyroid, prostate, and skin (including squamous cell carcinoma); hematopoietic tumors of lymphoid lineage (including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma); hematopoietic tumors of myeloid lineage (including acute and chronic myelogenous leukemias, myelodysplastic syndrome and promyelocytic leukemia); tumors of mesenchymal origin (including fibrosarcoma and rhabdomyosarcoma, and other sarcomas, e.g. soft tissue and bone); tumors of the central and peripheral nervous system (including astrocytoma, neuroblastoma, glioma and schwannomas); and other tumors (including melanoma, seminoma, teratocarcinoma, osteosarcoma, xenoderoma pigmentosum, keratoctanthoma, thyroid follicular cancer and Kaposi's sarcoma) The compounds are useful for the treatment of neoplasia selected from lung cancer, colon cancer and breast cancer.

The compounds would also be useful for treatment of ophthalmological conditions such as corneal graft rejection, ocular neovascularization, retinal neovascularization including neovascularization following injury or infection, diabetic retinopathy, retrolental fibroplasia and neovascular glaucoma; retinal ischemia; vitreous hemorrhage; ulcerative diseases such as gastric ulcer; pathological, but non-malignant, conditions such as hemangiomas, including infantile hemaginomas, angiofibroma of the nasopharynx and avascular necrosis of bone; and disorders of the female reproductive system such as endometriosis. The compounds are also useful for the treatment of edema, and conditions of vascular hyperpermeability.

Based on the ability to modulate kinases impacting angiogenesis, the compounds of the invention are also useful in treatment and therapy of proliferative diseases. Particularly, these compounds can be used for the treatment of an inflammatory rheumatoid or rheumatic disease, especially of manifestations at the locomotor apparatus, such as various inflammatory rheumatoid diseases, especially chronic polyarthritis including rheumatoid arthritis, juvenile arthritis or psoriasis arthropathy; paraneoplastic syndrome or tumor-induced inflammatory diseases, turbid effusions, collagenosis, such as systemic Lupus erythematosus, poly-myositis, dermato-myositis, systemic sclerodermia or mixed collagenosis; postinfectious arthritis (where no living pathogenic organism can be found at or in the affected part of the body), seronegative spondylarthritis, such as spondylitis ankylosans; vasculitis, sarcoidosis, or arthrosis; or further any combinations thereof. An example of an inflammation related disorder is (a) synovial inflammation, for example, synovitis, including any of the particular forms of synovitis, in particular bursal synovitis and purulent synovitis, as far as it is not crystal-induced. Such synovial inflammation may for example, be consequential to or associated with disease, e.g. arthritis, e.g. osteoarthritis, rheumatoid arthritis or arthritis deformans. The present invention is further applicable to the systemic treatment of inflammation, e.g. inflammatory diseases or conditions, of the joints or locomotor apparatus in the region of the tendon insertions and tendon sheaths. Such inflammation may be, for example, consequential to or associated with disease or further (in a broader sense of the invention) with surgical intervention, including, in particular conditions such as insertion endopathy, myofasciale syndrome and tendomyosis. The present invention is further applicable to the treatment of inflammation, e.g. inflammatory disease or condition, of connective tissues including dermatomyositis and myositis.

The compounds of the invention can also be used as active agents against such disease states as arthritis, atherosclerosis, psoriasis, hemangiomas, myocardial angiogenesis, coronary and cerebral collaterals, ischemic limb angiogenesis, wound healing, peptic ulcer Helicobacter related diseases, fractures, cat scratch fever, rubeosis, neovascular glaucoma and retinopathies such as those associated with diabetic retinopathy or macular degeneration. In addition, some of these compounds can be used as active agents against solid tumors, malignant ascites, hematopoietic cancers and hyperproliferative disorders such as thyroid hyperplasia (especially Grave's disease), and cysts (such as hypervascularity of ovarian stroma, characteristic of polycystic ovarian syndrome (Stein-Leventhal syndrome)) since such diseases require a proliferation of blood vessel cells for growth and/or metastasis.

The compounds of the invention can also be used as active agents against burns, chronic lung disease, stroke, polyps, anaphylaxis, chronic and allergic inflammation, ovarian hyperstimulation syndrome, brain tumor-associated cerebral edema, high-altitude, trauma or hypoxia induced cerebral or pulmonary edema, ocular and macular edema, ascites, and other diseases where vascular hyperpermeability, effusions, exudates, protein extravasation, or edema is a manifestation of the disease. The compounds will also be useful in treating disorders in which protein extravasation leads to the deposition of fibrin and extracellular matrix, promoting stromal proliferation (e.g. fibrosis, cirrhosis and carpal tunnel syndrome).

The compounds of the invention are also useful in the treatment of ulcers including bacterial, fungal, Mooren ulcers and ulcerative colitis.

The compounds of the invention are also useful in the treatment of conditions wherein undesired angiogenesis, edema, or stromal deposition occurs in viral infections such as Herpes simplex, Herpes Zoster, AIDS, Kaposi's sarcoma, protozoan infections and toxoplasmosis, following trauma, radiation, stroke, endometriosis, ovarian hyperstimulation syndrome, systemic lupus, sarcoidosis, synovitis, Crohn's disease, sickle cell anemia, Lyme disease, pemphigoid, Paget's disease, hyperviscosity syndrome, Osler-Weber-Rendu disease, chronic inflammation, chronic occlusive pulmonary disease, asthma, and inflammatory rheumatoid or rheumatic disease. The compounds are also useful in the reduction of sub-cutaneous fat and for the treatment of obesity. The compounds of the invention are also useful in the treatment of ocular conditions such as ocular and macular edema, ocular neovascular disease, scleritis, radial keratotomy, uveitis, vitritis, myopia, optic pits, chronic retinal detachment, post-laser complications, glaucoma, conjunctivitis, Stargardt's disease and Eales disease in addition to retinopathy and macular degeneration. The compounds of the invention are also useful in the treatment of cardiovascular conditions such as atherosclerosis, restenosis, arteriosclerosis, vascular occlusion and carotid obstructive disease.

The compounds of the invention are also useful in the treatment of cancer related indications such as solid tumors, sarcomas (especially Ewing's sarcoma and osteosarcoma), retinoblastoma, rhabdomyosarcomas, neuroblastoma, hematopoietic malignancies, including leukemia and lymphoma, tumor-induced pleural or pericardial effusions, and malignant ascites.

The compounds of the invention are also useful in the treatment of diabetic conditions such as diabetic retinopathy and microangiopathy.

The compounds of the present invention are also capable of inhibiting other protein kinase-associated disorders, and thus may be effective in the treatment of diseases associated with other protein kinases. “Protein tyrosine kinase-associated disorders” are those disorders which result from aberrant tyrosine kinase activity, and/or which are alleviated by the inhibition of one or more of these enzymes. For example, the compounds of the present invention inhibit the protein tyrosine kinase Lck, and are thus useful in the treatment, including prevention and therapy, of Lck-associated disorders such as immunologic disorders. Lck inhibitors are of value in the treatment of a number of such disorders (for example, the treatment of autoimmune diseases), as Lck inhibition blocks T cell activation. The treatment of T cell mediated diseases, including inhibition of T cell activation and proliferation, is a preferred embodiment of the present invention. Compounds of the present invention which selectively block T cell activation and proliferation are preferred. Also, compounds of the present invention which may block the activation of endothelial cell protein tyrosine kinase by oxidative stress, thereby limiting surface expression of adhesion molecules that induce neutrophil binding, and which can inhibit protein tyrosine kinase necessary for neutrophil activation would be useful, for example, in the treatment of ischemia and reperfusion injury.

The present invention also provides methods for the treatment of protein tyrosine kinase-associated disorders, comprising the step of administering to a subject in need thereof at least one compound of the Formula I or of Formula II in an amount effective therefor. Other therapeutic agents such as those described below may be employed with the inventive compounds in the present methods. In the methods of the present invention, such other therapeutic agent(s) may be administered prior to, simultaneously with or following the administration of the compound(s) of the present invention.

Use of the compound(s) of the present invention in treating protein tyrosine kinase-associated disorders is exemplified by, but is not limited to, treating a range of disorders such as: arthritis (such as rheumatoid arthritis, psoriatic arthritis or osteoarthritis); transplant (such as organ transplant, acute transplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; multiple sclerosis; inflammatory bowel disease, including ulcerative colitis and Crohn's disease; lupus (systemic lupus erythematosis); graft vs. host diseases; T-cell mediated hypersensitivity diseases, including contact hypersensitivity, delayed-type hypersensitivity, and gluten-sensitive enteropathy (Celiac disease); Type 1 diabetes; psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; cancers where Lck or other Src-family kinases such as Src are activated or overexpressed, such as colon carcinoma and thymoma, or cancers where Src-family kinase activity facilitates tumor growth or survival; glomerulonephritis, serum sickness; uticaria; allergic diseases such as respiratory allergies (asthma, hayfever, allergic rhinitis) or skin allergies; scleracielma; mycosis fungoides; acute inflammatory responses (such as acute respiratory distress syndrome and ishchemia/reperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic schlerosis; and morphea. The present invention also provides for a method for treating the aforementioned disorders such as atopic dermatitis by administration of a therapeutically effective amount of a compound of the present invention, which is an inhibitor of protein tyrosine kinase, to a patient in need of such treatment.

The combined activity of the present compounds towards monocytes, macrophages, T cells, etc. may prove to be a valuable tool in the treatment of any of the aforementioned disorders.

In a particular embodiment, the compounds of the present invention are useful for the treatment of the aforementioned exemplary disorders irrespective of their etiology, for example, for the treatment of rheumatoid arthritis, transplant rejection, multiple sclerosis, inflammatory bowel disease, lupus, graft v. host disease, T cell mediated hypersensitivity disease, psoriasis, Hashimoto's thyroiditis, Guillain-Barre syndrome, cancer, contact dermatitis, allergic disease such as allergic rhinitis, asthma, ischemic or reperfusion injury, or atopic dermatitis whether or not associated with PTK.

In another embodiment, the compounds are useful for the treatment of rheumatoid spondylitis, gouty arthritis, adult respiratory distress syndrome (ARDS), anaphylaxis, muscle degeneration, cachexia, Reiter's syndrome, type II diabetes, bone resorption diseases, graft vs. host reaction, Alzheimer's disease, atherosclerosis, brain trauma, multiple sclerosis, cerebral malaria, sepsis, septic shock, toxic shock syndrome, fever, and myalgias due to infection, or which subject is infected by HIV-1, HIV-2, HIV-3, cytomegalovirus (CMV), influenza, adenovirus, the herpes viruses (including HSV-1, HSV-2), or herpes zoster in a subject, the method comprising administering to the subject a pharmaceutical composition comprising an effective dosage amount of a compound according to any of Claims 1-18.

In yet another embodiment, the compounds are useful for decreasing the level of one or more of TNF-α, IL-1β, IL-6 and IL-8 in a subject, which is typically a human.

Besides being useful for human treatment, these compounds are useful for veterinary treatment of companion animals, exotic animals and farm animals, including mammals, rodents, and the like. For example, animals including horses, dogs, and cats may be treated with compounds provided by the invention.

### FORMULATIONS AND METHOD OF USE

Treatment of diseases and disorders herein is intended to also include therapeutic administration of a compound of the invention, or a pharmaceutical salt thereof, or a pharmaceutical composition of either to a subject (i.e., an animal, preferably a mammal, most preferably a human) which may be in need of preventative treatment, such as, for example, for pain, inflammation, cancer and the like. Treatment also encompasses prophylactic administration of a compound of the invention, or a pharmaceutical salt thereof, or a pharmaceutical composition of either to a subject (i.e., an animal, preferably a mammal, most preferably a human). Generally, the subject is initially diagnosed by a licensed physician and/or authorized medical practitioner, and a regimen for prophylactic and/or therapeutic treatment via administration of the compound(s) or compositions of the invention is suggested, recommended or prescribed.

While it may be possible to administer a compound of the invention alone, in the methods described, the compound administered normally will be present as an active ingredient in a pharmaceutical composition. Thus, in another embodiment of the invention, there is provided a pharmaceutical composition comprising a compound of this invention in combination with a pharmaceutically acceptable carrier, which includes diluents, excipients, adjuvants and the like (collectively referred to herein as “carrier” materials) as described herein, and, if desired, other active ingredients. A pharmaceutical composition of the invention may comprise an effective amount of a compound of the invention or an effective dosage amount of a compound of the invention. An effective dosage amount of a compound of the invention includes an amount less than, equal to or greater than an effective amount of the compound; for example, a pharmaceutical composition in which two or more unit dosages, such as in tablets, capsules and the like, are required to administer an effective amount of the compound, or alternatively, a multi-dose pharmaceutical composition, such as powders, liquids and the like, in which an effective amount of the compound is administered by administering a portion of the composition.

The compound(s) of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, and typically from about 1 to 500 mg. A suitable daily dose for a human or other mammal may vary widely depending on the condition of the patient and other factors, but, once again, can be determined using routine methods and practices.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg, advantageously between about 0.01 and about 50 mg/kg, and more advantageously about 0.01 and about 30 mg/kg body weight may be appropriate. The daily dose can be administered in one to four doses per day.

For therapeutic purposes, the active compounds of this invention are ordinarily combined with one or more adjuvants or “excipients” appropriate to the indicated route of administration. If administered on a per dose basis, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, to form the final formulation. For example, the active compound(s) and excipient(s) may be tableted or encapsulated by known and accepted methods for convenient administration. Examples of suitable formulations include, without limitation, pills, tablets, soft and hard-shell gel capsules, troches, orally-dissolvable forms and delayed or controlled-release formulations thereof. Particularly, capsule or tablet formulations may contain one or more controlled-release agents, such as hydroxypropylmethyl cellulose, as a dispersion with the active compound(s).

In the case of psoriasis and other skin conditions, it may be preferable to apply a topical preparation of compounds of this invention to the affected area two to four times a day.

Formulations suitable for topical administration include liquid or semi-liquid preparations suitable for penetration through the skin (e.g., liniments, lotions, ointments, creams, pastes, suspensions and the like) and drops suitable for administration to the eye, ear, or nose. A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

When formulated in an ointment, the active ingredients may be employed with either paraffinic or a water-miscible ointment base. Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound, which enhances absorption or penetration of the active ingredient through the skin or other affected areas. Examples of such dermal penetration enhancers include DMSO and related analogs.

The compounds of this invention can also be administered by transdermal device. Preferably transdermal administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizers) make-up the so-called emulsifying wax, and the wax together with the oil and fat make up the so-called emulsifying ointment base, which forms the oily dispersed phase of the cream formulations. Emulsifiers and emulsion stabilizers suitable for use in the formulation of the present invention include, for example, Tween 60, Span 80, cetostearyl alcohol, myristyl alcohol, glyceryl monostearate, sodium lauryl sulfate, glyceryl distearate alone or with a wax, or other materials well known in the art.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The active ingredients are preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables. The active ingredient may also be administered by injection as a composition with suitable carriers including saline, dextrose, or water. The daily parenteral dosage regimen will be from about 0.1 to about 30 mg/kg of total body weight, preferably from about 0.1 to about 10 mg/kg, and more preferably from about 0.25 mg to 1 mg/kg.

For pulmonary administration, the pharmaceutical composition may be administered in the form of an aerosol or with an inhaler including dry powder aerosol.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

### Combinations

While the compounds of the invention can be dosed or administered as the sole active pharmaceutical agent, they can also be used in combination with one or more compounds of the invention or in conjunction with other agents. When administered as a combination, the therapeutic agents can be formulated as separate compositions that are administered simultaneously or sequentially at different times, or the therapeutic agents can be given as a single composition.

The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent.

Specifically, the administration of compounds of the present invention may be in conjunction with additional therapies known to those skilled in the art in the prevention or treatment of neoplasia, such as with radiation therapy or with cytostatic or cytotoxic agents.

If formulated as a fixed dose, such combination products employ the compounds of this invention within the accepted dosage ranges. Compounds of Formulae I and II may also be administered sequentially with known anticancer or cytotoxic agents when a combination formulation is inappropriate. The invention is not limited in the sequence of administration; compounds of the invention may be administered either prior to, simultaneous with or after administration of the known anticancer or cytotoxic agent.

Currently, standard treatment of primary tumors consists of surgical excision followed by either radiation or IV administered chemotherapy. The typical chemotherapy regime consists of either DNA alkylating agents, DNA intercalating agents, CDK inhibitors, or microtubule poisons. The chemotherapy doses used are just below the maximal tolerated dose and therefore dose limiting toxicities typically include, nausea, vomiting, diarrhea, hair loss, neutropenia and the like.

There are large numbers of antineoplastic agents available in commercial use, in clinical evaluation and in pre-clinical development, which would be selected for treatment of neoplasia by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents.

A first family of antineoplastic agents, which may be used in combination with compounds of the invention consists of antimetabolite-type/thymidilate synthase inhibitor antineoplastic agents. Suitable antimetabolite antineoplastic agents may be selected from but not limited to the group consisting of 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT and uricytin.

A second family of antineoplastic agents, which may be used in combination with compounds of the invention consists of alkylating-type antineoplastic agents. Suitable alkylating-type antineoplastic agents may be selected from but not limited to the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

A third family of antineoplastic agents which may be used in combination with compounds of the invention consists of antibiotic-type antineoplastic agents. Suitable antibiotic-type antineoplastic agents may be selected from but not limited to the group consisting of Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

A fourth family of antineoplastic agents which may be used in combination with compounds of the invention consists of a miscellaneous family of antineoplastic agents, including tubulin interacting agents, topoisomerase II inhibitors, topoisomerase I inhibitors and hormonal agents, selected from but not limited to the group consisting of α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides and Yamanouchi YM-534.

Alternatively, the compounds of the invention may also be used in co-therapies with other anti-neoplastic agents, such as other kinase inhibitors including p38 inhibitors and CDK inhibitors, TNF inhibitors, metallomatrix proteases inhibitors (MMP), COX-2 inhibitors including celecoxib, rofecoxib, parecoxib, valdecoxib, and etoricoxib, NSAID's, SOD mimics or αᵥβ₃ inhibitors.

The foregoing description is merely illustrative of the invention and is not intended to limit the invention to the disclosed compounds, compositions and methods. Variations and changes, which are obvious to one skilled in the art, are intended to be within the scope and nature of the invention, as defined in the appended claims. From the foregoing description, one skilled in the art can easily ascertain the essential characteristics of this invention, and without departing from the spirit and scope thereof, can make various changes and modifications of the invention to adapt it to various usages and conditions. All patents and other publications recited herein are hereby incorporated by reference in their entireties.

What is claimed is:

1. A compound of the Formula I: 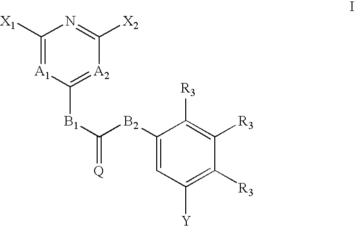
or stereoisomer, tautomer, solvate, pharmaceutically acceptable salt, derivative or prodrug thereof, wherein
A¹ is CH or N;
A² is CH or N;
B¹ is NR², O or S;
B² is NR², O or S;
Q is O, S, NH or N(CN);
one X¹ and X² is H, halo, NO₂, CN, NR¹R², NH₂, OR¹, SR¹, C(O)NR¹R², C(O)R⁶ or (CH₂)ₙR⁶ and the other of X¹ and X² is H;
alternatively, when A¹ is C and X¹ is N or CH, then A¹ and X¹ taken together may form a 5-6-membered unsaturated ring formed of carbons atoms and optionally comprising 1-3 heteroatoms selected from N, O and S, said ring optionally substituted with 1-3 substituents of R⁶, provided that the fused hetero bicyclic ring thus formed is not quinoline or 1,5-naphthydrine;
Y is C(O)R⁵, S(O)₂R⁵, NR⁴R⁵, C(O)NR⁴R⁴, C(O)NH⁴R⁵, COOR⁵, NR⁴C(O)R⁵, S(O)₂NR⁴R⁴, S(O)₂NR⁴R⁵ or NR⁴S(O)₂R⁵;
R¹ is C₁₋₁₀-alkyl, C₂₋₁₀-alkenyl, C₂₋₁₀-alkynyl or C₃₋₇-cycloalkyl, each of the C₁₋₁₀-alkyl, C₂₋₁₀-alkenyl, C₂₋₁₀-alkynyl and C₃₋₇-cycloalkyl optionally substituted with one or more substituents of R⁶, or R¹ is R⁶;
R² is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;
each R³, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁵ or R⁶;
alternatively any two adjacent R³'s taken together form a saturated or partially or fully unsaturated 5-6 membered monocyclic ring of carbon atoms optionally including 1-3 heteroatoms selected from O, N, or S, the ring optionally substituted independently with 1-3 substituents of R⁵ or R⁶;
each R⁴, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;
R⁵ is a partially or fully saturated or unsaturated 3-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, and wherein each ring of said ring system is optionally substituted independently with 1-3 substituents of R⁶, oxo, NR⁶R⁶, OR⁶, SR⁶, C(O)R⁶, COOR⁶, C(O)NR⁶R⁶, NR⁶C(O)R⁶, NR⁶C(O)NR⁶R⁶, OC(O)NR⁶R⁶, S(O)₂R⁶, S(O)₂NR⁶R⁶ or NR⁶S(O)₂R⁶;
each R⁶, independently, is H, oxo, halo, haloalkyl, CN, OH, NO₂, NH₂, acetyl, C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl or a saturated or partially or fully unsaturated 3-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, wherein each of the C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl and ring of said ring system is optionally substituted independently with 1-3 substituents of halo, haloalkyl, CN, NO₂, NH₂, OH, oxo, methyl, methoxyl, ethyl, ethoxyl, propyl, propoxyl, isopropyl, cyclopropyl, butyl, isobutyl, tert-butyl, methylamine, dimethylamine, ethylamine, diethylamine, propylamine, isopropylamine, dipropylamine, diisopropylamine, benzyl or phenyl; and
n is 0, 1, 2, 3 or 4.

2. The compound of claim 1 wherein A¹ is CH and A² is N.

3. The compound of claim 1 wherein A¹ is N and A² is CH.

4. The compound of claim 1 wherein A¹ is N and A² is N.

5. The compound of claim 1 wherein Q is O, B¹ is NR² and B² is NH.

6. The compound of claim 1 wherein one of X¹ and X² is NR¹R² or NH₂ and the other of X¹ and X² is H.

7. The compound of claim 1 wherein both of X¹ and X² are H.

8. The compound of claim 1 wherein Y is NR⁴R⁵, C(O)NR⁴R⁴, C(O)NR⁴R⁵, NR⁴C(O)R⁵, S(O)₂NR⁴R⁴, S(O)₂NR⁴R⁵ or NR⁴S(O)₂R⁵.

9. The compound of claim 1 wherein R⁵ is phenyl, naphthyl, pyridyl, pyrimidyl, pyridazinyl, pyrazinyl, triazinyl, quinolinyl, isoquinolinyl, quinazolinyl, isoquinazolinyl, aza-quinazolinyl, phthalazinyl, aza-phthalazinyl, thiophenyl, furyl, pyrrolyl, pyrazolyl, imidazolyl, triazolyl, thiazolyl, oxazolyl, isoxazolyl, isothiazolyl, indolyl, isoindolyl, indolinyl, benzofuranyl, benzothiophenyl, benzimidazolyl, benzoxazolyl, benzisoxazolyl, benzothiazolyl, benzoisothiazolyl, benzotriazolyl, tetrahydrofuranyl, pyrrolidinyl, oxazolinyl, isoxazolinyl, thiazolinyl, pyrazolinyl, morpholinyl, piperidinyl, piperazinyl, pyranyl, dioxozinyl, cyclopropyl, cyclobutyl, cyclopentyl, cyclohexyl and cycloheptyl, each ring of which is optionally substituted independently with one or more substituents of R⁶.

10. The compound of claim 1 selected from
4-methyl-3-(((methyl(6-(methylamino)-4-pyrimidinyl)amino)carbonyl)amino)-N-(3-(trifluoromethyl)phenyl)benzamide;
4-methyl-N-(3-(1-methylethyl)phenyl)-3-(((methyl(6-(methylamino)-4-pyrimidinyl)amino)carbonyl)amino)benzamide;
4-methyl-3-(((methyl(6-(methylamino)-4-pyrimidinyl)amino)carbonyl)amino)-N-(3-(methyloxy)-5-(trifluoromethyl)phenyl)benzamide;
4-methyl-3-(((methyl(6-(methylamino)-4-pyrimidinyl)amino)carbonyl)amino)-N-(3-methyl-4-(1-methylethyl)phenyl)benzamide;
4-methyl-3-(((methyl(6-(methylamino)-4-pyrimidinyl)amino)carbonyl)amino)-N-(2-methyl-3-(trifluoromethyl)phenyl)benzamide;
N-(1-acetyl-3,3-dimethyl-2,3-dihydro-1H-indol-6-yl)-4-methyl-3-(((methyl(6-(methylamino)-4-pyrimidinyl)amino)carbonyl)amino)benzamide;
4-methyl-N-(3-(1-methylethyl)phenyl)-3-((((3-(4-morpholinyl)propyl)(4-pyrimidinyl)amino)carbonyl)amino)benzamide;
4-methyl-3-(((methyl(6-(methylamino)-4-pyrimidinyl)amino)carbonyl)amino)-N-(2-naphthalenyl)benzamide;
N-(2-fluoro-3-(trifluoromethyl)phenyl)-4-methyl-3-(((methyl(6-(methylamino)-4-pyrimidinyl)amino)carbonyl)amino)benzamide;
N-(4-(1,1-dimethylethyl)phenyl)-4-methyl-3-(((methyl(6-(methylamino)-4-pyrimidinyl)amino)carbonyl)amino)benzamide;
N-(3-(dimethylamino)phenyl)-4-methyl-3-(((methyl(6-(methylamino)-4-pyrimidinyl)amino)carbonyl)amino)benzamide;
N-(4-(1,1-dimethylethyl)phenyl)-4-methyl-3-(((methyl(4-(methylamino)-1,3,5-triazin-2-yl)amino)carbonyl)amino)benzamide;
N-(3-(dimethylamino)phenyl)-4-methyl-3-(((methyl(4-(methylamino)-1,3,5-triazin-2-yl)amino)carbonyl)amino)benzamide;
4-methyl-3-((((2-(methylamino)-4-pyrimidinyl)(3-(4-morpholinyl)propyl)amino)carbonyl)amino)-N-(3-(1-methylethyl)phenyl)benzamide;
4-methyl-3-(((methyl(4-(methylamino)-1,3,5-triazin-2-yl)amino)carbonyl)amino)-N-(2-naphthalenyl)benzamide;
N-(1,1′-biphenyl-3-yl)-4-methyl-3-(((methyl(6-(methylamino)-4-pyrimidinyl)amino)carbonyl)amino)benzamide;
4-methyl-3-(((methyl(6-(methylamino)-4-pyrimidinyl)amino)carbonyl)amino)-N-(4-(trifluoromethyl)phenyl)benzamide;
4-methyl-N-(2-methyl-1,3-benzothiazol-5-yl)-3-(((methyl(6-(methylamino)-4-pyrimidinyl)amino)carbonyl)amino)benzamide;
4-methyl-3-(((methyl(6-(methylamino)-4-pyrimidinyl)amino)carbonyl)amino)-N-(2-((2-(1-pyrrolidinyl)ethyl)oxy)-5-(trifluoromethyl)phenyl)benzamide;
4-methyl-N-(2-methyl-1,3-benzothiazol-5-yl)-3-(((methyl(4-(methylamino)-1,3,5-triazin-2-yl)amino)carbonyl)amino)benzamide;
4-methyl-3-(((methyl(4-(methylamino)-1,3,5-triazin-2-yl)amino)carbonyl)amino)-N-(3-(methyloxy)-5-(trifluoromethyl)phenyl)benzamide;
4-methyl-3-(((methyl(4-(methylamino)-1,3,5-triazin-2-yl)amino)carbonyl)amino)-N-(2-methyl-3-(trifluoromethyl)phenyl)benzamide;
4-methyl-3-(((methyl(4-(methylamino)-1,3,5-triazin-2-yl)amino)carbonyl)amino)-N-(3-methyl-4-(1-methylethyl)phenyl)benzamide;
N-(1,1′-biphenyl-3-yl)-4-methyl-3-(((methyl(4-(methylamino)-1,3,5-triazin-2-yl)amino)carbonyl)amino)benzamide;
N-(4-chlorophenyl)-4-methyl-3-(((methyl(6-(methylamino)-4-pyrimidinyl)amino)carbonyl)amino)benzamide;
4-methyl-3-(((methyl(6-(methylamino)-4-pyrimidinyl)amino)carbonyl)amino)-N-phenylbenzamide;
N-butyl-4-methyl-3-(((methyl(6-(methylamino)-4-pyrimidinyl)amino)carbonyl)amino)benzamide;
N-(5-cyclohexyl-2-(methyloxy)phenyl)-4-methyl-3-(((methyl(6-(methylamino)-4-pyrimidinyl)amino)carbonyl)amino)benzamide;
4-methyl-3-(((methyl(4-(methylamino)-1,3,5-triazin-2-yl)amino)carbonyl)amino)-N-phenylbenzamide;
4-methyl-3-(((methyl(6-(methylamino)-4-pyrimidinyl)amino)carbonyl)amino)-N-(phenylmethyl)benzamide;
N-(5-cyclohexyl-2-(methyloxy)phenyl)-4-methyl-3-((((3-(4-morpholinyl)propyl)(4-pyrimidinyl)amino)carbonyl)amino)benzamide;
N-(4-chlorophenyl)-4-methyl-3-(((methyl(4-(methylamino)-1,3,5-triazin-2-yl)amino)carbonyl)amino)benzamide;
N-butyl-4-methyl-3-(((methyl(4-(methylamino)-1,3,5-triazin-2-yl)amino)carbonyl)amino)benzamide;
4-methyl-3-(((methyl(4-(methylamino)-1,3,5-triazin-2-yl)amino)carbonyl)amino)-N-(phenylmethyl)benzamide;
N-(5-cyclohexyl-2-(methyloxy)phenyl)-4-methyl-3-((((2-(methylamino)-4-pyrimidinyl)(3-(4-morpholinyl)propyl)amino)carbonyl)amino)benzamide;
N-methyl-4-(methyl(((2-methyl-5-(((phenylmethyl)amino)carbonyl)phenyl)amino)carbonyl)amino)-2-pyridinecarboxamide;
N-methyl-N-(6-(methylamino)-4-pyrimidinyl)-N′-(2-methyl-5-((7-(trifluoromethyl)-3,4-dihydro-1(2H)-quinolinyl)carbonyl)phenyl)urea;
N-(2,6-dichlorophenyl)-4-methyl-3-(((methyl(6-(methylamino)-4-pyrimidinyl)amino)carbonyl)amino)benzamide;
3-((((6-(formyl(methyl)amino)-4-pyrimidinyl)(methyl)amino)carbonyl)amino)-4-methyl-N-(2-(1-piperidinyl)-5-(trifluoromethyl)phenyl)benzamide;
4-methyl-3-(((methyl(6-(methylamino)-4-pyrimidinyl)amino)carbonyl)amino)-N-(2-(1-piperidinyl)-5-(trifluoromethyl)phenyl)benzamide;
N-methyl-4-(methyl(((2-methyl-5-((phenylamino)carbonyl)phenyl)amino)carbonyl)amino)-2-pyridinecarboxamide;
4-((((5-(((4-chlorophenyl)amino)carbonyl)-2-methylphenyl)amino)carbonyl)(methyl)amino)-N-methyl-2-pyridinecarboxamide;
4-((((5-(((4-(1,1-dimethylethyl)phenyl)amino)carbonyl)-2-methylphenyl)amino)carbonyl)(methyl)amino)-N-methyl-2-pyridinecarboxamide;
N-methyl-4-(methyl(((2-methyl-5-(((3-methyl-4-(1-methylethyl)phenyl)amino)carbonyl)phenyl)amino)carbonyl)amino)-2-pyridinecarboxamide;
N-methyl-4-(methyl(((2-methyl-5-(((4-(trifluoromethyl)phenyl)amino)carbonyl)phenyl)amino)carbonyl)amino)-2-pyridinecarboxamide;
N-methyl-4-(methyl(((2-methyl-5-(((3-(trifluoromethyl)phenyl)amino)carbonyl)phenyl)amino)carbonyl)amino)-2-pyridinecarboxamide;
N-methyl-4-(methyl(((2-methyl-5-(((2-methyl-1,3-benzothiazol-5-yl)amino)carbonyl)phenyl)amino)carbonyl)amino)-2-pyridinecarboxamide;
4-((((5-((1,1′-biphenyl-3-ylamino)carbonyl)-2-methylphenyl)amino)carbonyl)(methyl)amino)-N-methyl-2-pyridinecarboxamide;
4-((((5-(((3-(dimethylamino)phenyl)amino)carbonyl)-2-methylphenyl)amino)carbonyl)(methyl)amino)-N-methyl-2-pyridinecarboxamide;
4-((((5-(((1-acetyl-3,3-dimethyl-2,3-dihydro-1H-indol-6-yl)amino)carbonyl)-2-methylphenyl)amino)carbonyl)(methyl)amino)-N-methyl-2-pyridinecarboxamide;
4-((((5-(((2-fluoro-3-(trifluoromethyl)phenyl)amino)carbonyl)-2-methylphenyl)amino)carbonyl)(methyl)amino)-N-methyl-2-pyridinecarboxamide;
N-methyl-4-(methyl(((2-methyl-5-(((3-(methyloxy)-5-(trifluoromethyl)phenyl)amino)carbonyl)phenyl)amino)carbonyl)amino)-2-pyridinecarboxamide;
4-((((5-(((2,6-dichlorophenyl)amino)carbonyl)-2-methylphenyl)amino)carbonyl)(methyl)amino)-N-methyl-2-pyridinecarboxamide;
N-methyl-4-(methyl(((2-methyl-5-((2-naphthalenylamino)carbonyl)phenyl)amino)carbonyl)amino)-2-pyridinecarboxamide;
N-methyl-4-(methyl(((2-methyl-5-(((2-methyl-3-(trifluoromethyl)phenyl)amino)carbonyl)phenyl)amino)carbonyl)amino)-2-pyridinecarboxamide; and
N-methyl-4-(methyl(((2-methyl-5-((7-(trifluoromethyl)-3,4-dihydro-1(2H)-quinolinyl)carbonyl)phenyl)amino)carbonyl)amino)-2-pyridinecarboxamide.

11. A compound of the Formula II: 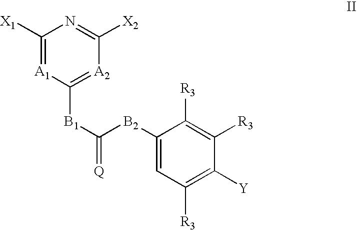
or stereoisomer, tautomer, solvate, pharmaceutically acceptable salt, derivative or prodrug thereof, wherein
A¹ is CH or N;
A² is CH or N;
B¹ is NR², O or S;
B² is NR², O or S;
Q is O, S, NH or N(CN);
one X¹ and X² is H, halo, NO₂, CN, NR¹R², NH₂, OR¹, SR¹, C(O)NR¹R², C(O)R⁶ or (CH₂)ₙR⁶ and the other of X¹ and X² is H;
alternatively, when A¹ is C and X¹ is N or CH, then A¹ and X¹ taken together may form a 5-6-membered unsaturated ring formed of carbons atoms and optionally comprising 1-3 heteroatoms selected from N, O and S, said ring optionally substituted with 1-3 substituents of R⁶;
Y is C(O)R⁵, S(O)₂R⁵, NR⁴R⁵, C(O)NR⁴R⁴, C(O)NR⁴R⁵, COOR⁵, NR⁴C(O)R⁵, S(O)₂NR⁴R⁴, S(O)₂NR⁴R⁵ or NR⁴S(O)₂R⁵;
R¹ is C₁₋₁₀-alkyl, C₂₋₁₀-alkenyl, C₂₋₁₀-alkynyl or C₃₋₇-cycloalkyl, each of the C₁₋₁₀-alkyl, C₂₋₁₀-alkenyl, C₂₋₁₀-alkynyl and C₃₋₇-cycloalkyl optionally substituted with one or more substituents of R⁶, or R¹ is R⁶;
R² is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;
each R³, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁵ or R⁶;
alternatively any two adjacent R³'s taken together form a saturated or partially or fully unsaturated 5-6 membered monocyclic ring of carbon atoms optionally including 1-3 heteroatoms selected from O, N, or S, the ring optionally substituted independently with 1-3 substituents of R⁵ or R⁶;
each R⁴, independently, is H, C₁₋₁₀-alkyl, C₂₋₁₀-alkenyl or C₂₋₁₀-alkynyl, each of the C₁₋₁₀-alkyl, C₂₋₁₀-alkenyl and C₂₋₁₀-alkynyl optionally comprising 1-3 heteroatoms selected from N, O and S and optionally substituted with one or more substituents of R⁶;
R⁵ is a partially or fully saturated or unsaturated 3-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, and wherein each ring of said ring system is optionally substituted independently with 1-3 substituents of R⁶, oxo, NR⁶R⁶, OR⁶, SR⁶, C(O)R⁶, COOR⁶, C(O)NR⁶R⁶, NR⁶C(O)R⁶, NR⁶C(O)NR⁶R⁶, OC(O)NR⁶R⁶, S(O)₂R⁶, S(O)₂NR⁶R⁶ or NR⁶S(O)₂R⁶;
each R⁶, independently, is H, oxo, halo, haloalkyl, CN, OH, NO₂, NH₂, acetyl, C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl or a saturated or partially or fully unsaturated 3-8 membered monocyclic, 6-12 membered bicyclic, or 7-14 membered tricyclic ring system, said ring system formed of carbon atoms optionally including 1-3 heteroatoms if monocyclic, 1-6 heteroatoms if bicyclic, or 1-9 heteroatoms if tricyclic, said heteroatoms selected from O, N, or S, wherein each of the C₁₋₁₀-alkyl, C₂₋₁₀-alkenyl, C₂₋₁₀-alkynyl, C₃₋₁₀-cycloalkyl, C₄₋₁₀-cycloalkenyl, C₁₋₁₀-alkylamino-, C₁₋₁₀-dialkylamino-, C₁₋₁₀-alkoxyl, C₁₋₁₀-thioalkoxyl and ring of said ring system is optionally substituted independently with 1-3 substituents of halo, haloalkyl, CN, NO₂, NH₂, OH, oxo, acetyl, methyl, methoxyl, ethyl, ethoxyl, propyl, propoxyl, isopropyl, cyclopropyl, butyl, isobutyl, tert-butyl, methylamine, dimethylamine, ethylamine, diethylamine, propylamine, isopropylamine, dipropylamine, diisopropylamine, benzyl or phenyl; and
n is 0, 1, 2, 3 or 4,
provided that when Y is C(O)NR⁴R⁵ and R⁵ is phenyl, then the phenyl ring is not di-meta substituted with C(O)NR⁶R⁶.

12. The compound of claim 11 wherein A¹ is CH and A² is N.

13. The compound of claim 11 wherein A¹ is N and A² is CH.

14. The compound of claim 11 wherein A¹ is N and A² is N.

15. The compound of claim 11 wherein Q is O, B¹ is NR² and B² is NH.

16. The compound of claim 15 wherein one of X¹ and X² is NR¹R² or NH₂ and the other of X¹ and X² is H.

17. The compound of claim 15 wherein both of X¹ and X² are H.

18. The compound of claim 15 wherein Y is NR⁴R⁵, C(O)NR⁴R⁴, C(O)NR⁴R⁵, NR⁴C(O)R⁵, S(O)₂NR⁴R⁴, S(O)₂NR⁴R⁵ or NR⁴S(O)₂R⁵.

19. The compound of claim 11 wherein R⁵ is phenyl, naphthyl, pyridyl, pyrimidyl, pyridazinyl, pyrazinyl, triazinyl, quinolinyl, isoquinolinyl, quinazolinyl, isoquinazolinyl, aza-quinazolinyl, phthalazinyl, aza-phthalazinyl, thiophenyl, furyl, pyrrolyl, pyrazolyl, imidazolyl, triazolyl, thiazolyl, oxazolyl, isoxazolyl, isothiazolyl, indolyl, isoindolyl, indolinyl, benzofuranyl, benzothiophenyl, benzimidazolyl, benzoxazolyl, benzisoxazolyl, benzothiazolyl, benzoisothiazolyl, benzotriazolyl, tetrahydrofuranyl, pyrrolidinyl, oxazolinyl, isoxazolinyl, thiazolinyl, pyrazolinyl, morpholinyl, piperidinyl, piperazinyl, pyranyl, dioxozinyl, cyclopropyl, cyclobutyl, cyclopentyl, cyclohexyl and cycloheptyl, each ring of which is optionally substituted independently with one or more substituents of R⁶.

20. A pharmaceutical composition comprising a pharmaceutically acceptable carrier and an effective dosage amount of a compound of claim 1.

21. A pharmaceutical composition comprising a pharmaceutically acceptable carrier and an effective dosage amount of a compound of claim 11.

22. A method of modulating a protein kinase enzyme in a subject, the method comprising administering to the subject an effective dosage amount of a compound of claim 1.

23. The method of claim 22 wherein the kinase enzyme is KDR, Lck, p38 or Tie2.

24. A method of modulating a protein kinase enzyme in a subject, the method comprising administering to the subject an effective dosage amount of a compound of claim 11.

25. The method of claim 24 where in the kinase enzyme is KDR, Lck, p38 or Tie2.

26. A method of treating cancer in a subject, the method comprising administering to the subject an effective dosage amount of a compound of claim 1.

27. A method of treating cancer in a subject, the method comprising administering to the subject an effective dosage amount of a compound of claim 11.

28. A method of treating cancer in a subject, the method comprising administering to the subject an effective dosage amount of a pharmaceutical composition of claim 20.

29. A method of treating angiogenesis in a subject, the method comprising administering to the subject an effective dosage amount of a compound of claim 1.

30. A method of treating angiogenesis in a subject, the method comprising administering to the subject an effective dosage amount of a compound of claim 11.

31. A method of treating angiogenesis in a subject, the method comprising administering to the subject an effective dosage amount of a pharmaceutical composition of claim 20.

32. A method of treating a disorder related to at least one of Tie-2, KDR, p38 and Lck in a subject, the method comprising administering to the subject an effective dosage amount of a compound of claim 1.

33. A method of treating a disorder related to at least one of Tie-2, KDR, p38 and Lck in a subject, the method comprising administering to the subject an effective dosage amount of a compound of claim 11.

34. A method of treating a poliferative disorder in a subject, the method comprising administering to the subject an effective dosage amount of a compound of claim 1.

35. The method of claim 34 wherein the disorder is selected from the group consisting of myocardial infarction, coronary artery disease, peripheral vascular disease, stroke, ocular neovascularization, retinopathy, age-related macular degeneration, psoriasis, hemangioblastoma, hemangioma, arteriosclerosis, inflammatory disease rheumatoid arthritis, asthma, arterial or post-transplantational atherosclerosis, endometriosis, leukemia and combinations thereof.

36. A method of treating a proliferative disorder in a subject, the method comprising administering to the subject an effective dosage amount of a pharmaceutical composition of claim 20.

37. The method of claim 36 wherein the disorder is selected from the group consisting of myocardial infarction, coronary artery disease, peripheral vascular disease, stroke, ocular neovascularization, retinopathy, age-related macular degeneration, psoriasis, hemangioblastoma, hemangioma, arteriosclerosis, inflammatory disease rheumatoid arthritis, asthma, arterial or post-transplantational atherosclerosis, endometriosis, leukemia and combinations thereof.
